SEQUENCE LISTING (4 sequences)

SEQ ID NO: 1 - protein, 397 aa, Saccharomyces cerevisiae
MSPLRKTVPEFLAHLKSLPISKIASNDVLTICVGNESADMDSIASAITYSYCQYIYNEGTYSEEKKKGSFIVPIIDIPREDLSLRRDVMYVLEKLKIKEEELFFIEDLKSLKQNVSQGTELNSYLVDNNDTPKNLKNYIDNVVGIIDHHFDLQKHLDAEPRIVKVSGSCSSLVFNYWYEKLQGDREVVMNIAPLLMGAILIDTSNMRRKVEESDKLAIERCQAVLSGAVNEVSAQGLEDSSEFYKEIKSRKNDIKGFSVSDILKKDYKQFNFQGKGHKGLEIGLSSIVKRMSWLFNEHGGEADFVNQCRRFQAERGLDVLVLLTSWRKAGDSHRELVILGDSNVVRELIERVSDKLQLQLFGGNLDGGVAMFKQLNVEATRKQVVPYLEEAYSNLEE

SEQ ID NO: 2 - protein, 513 aa, Escherichia coli
MPIHDKSPRPQEFAAVDLGSNSFHMVIARVVDGAMQIIGRLKQRVHLADGLGPDNMLSEEAMTRGLNCLSLFAERLQGFSPASVCIVGTHTLRQALNATDFLKRAEKVIPYPIEIISGNEEARLIFMGVEHTQPEKGRKLVIDIGGGSTELVIGENFEPILVESRRMGCVSFAQLYFPGGVINKENFQRARMAAAQKLETLTWQFRIQGWNVAMGASGTIKAAHEVLMEMGEKDGIITPERLEKLVKEVLRHRNFASLSLPGLSEERKTVFVPGLAILCGVFDALAIRELRLSDGALREGVLYEMEGRFRHQDVRSRTASSLANQYHIDSEQARRVLDTTMQMYEQWREQQPKLAHPQLEALLRWAAMLHEVGLNINHSGLHRHSAYILQNSDLPGFNQEQQLMMATLVRYHRKAIKLDDLPRFTLFKKKQFLPLIQLLRLGVLLNNQRQATTTPPTLTLITDDSHWTLRFPHDWFSQNALVLLDLEKEQEYWEGVAGWRLKIEEESTPEIAA

SEQ ID NO: 3 - protein, 674 aa, Saccharomyces cerevisiae
MVVVGKSEVRNVSMSRPKKKSLIAILSTCVLFFLVFIIGAKFQYVSVFSKFLDDRGDNESLQLLNDIEFTRLGLTPREPVIIKDVKTGKERKLHGRFLHITDIHPDPYYVEGSSIDAVCHTGKPSKKKDVAPKFGKAMSGCDSPVILMEETLRWIKENLRDKIDFVIWTGDNIRHDNDRKHPRTEAQIFDMNNIVADKMTELFSAGNEEDPRDFDVSVIPSLGNNDVFPHNMFALGPTLQTREYYRIWKNFVPQQQQRTFDRSASFLTEVIPGKLAVLSINTLYLFKANPLVDNCNSKKEPGYQLLLWFGYVLEELRSRGMKVWLSGHVPPIAKNFDQSCYDKFTLWTHEYRDIIIGGLYGHMNIDHFIPTDGKKARKSLLKAMEQSTRVQQGEDSNEEDEETELNRILDHAMAAKEVFLMGAKPSNKEAYMNTVRDTYYRKVWNKLERVDEKNVENEKKKKEKKDKKKKKPITRKELIERYSIVNIGGSVIPTFNPSFRIWEYNITDIVNDSNFAVSEYKPWDEFFESLNKIMEDSLLEDEMDSSNIEVGINREKMGEKKNKKKKKNDKTMPIEMPDKYELGPAYVPQLFTPTRFVQFYADLEKINQELHNSFVESKDIFRYEIEYTSDEKPYSMDSLTVGSYLDLAGRLYENKPAWEKYVEWSFASSGYKDD

SEQ ID NO: 4 - protein, 172 aa, Homo sapiens
MMKLKSNQTRTYDGDGYKKRAACLCFRSESEEEVLLVSSSRHPDRWIVPGGGMEPEEEPSVAAVREVCEEAGVKGTLGRLVGIFENQERKHRTYVYVLIVTEVLEDWEDSVNIGRKREWFKIEDAIKVLQYHKPVQASYFETLRQGYSANNGTPVVATTYSVSAQSSMSGIR

(19) United States
(12) Patent Application Publication
(10) Pub. No.: US 2025/0327046 A1
(43) Pub. Date: Oct. 23, 2025

(54) ROLE OF POLYPHOSPHATES IN NEUROLOGICAL DISORDERS

(71) Applicant: UNIVERSITY OF MASSACHUSETTS, Boston, MA (US)

(72) Inventors: Brigitte Adriana Johanna VAN ZUNDERT, Vitacura-Santiago (CL); Robert H. BROWN, Jr., Needham, MA (US); Martin Alejandro MONTECINO, Vitacura-Santiago (CL); Cristian Andres ARREDONDO, Nunoa-Santiago (CL); Armando Francisco Antonio AMARO, Estacion Central-Santiago (CL); Carolina CEFALIELLO, Woods Hole, MA (US)

(21) Appl. No.: 18/706,494

(22) PCT Filed: Nov. 1, 2022
(86) PCT No.: PCT/US22/48584

§ 371 Date: May 1, 2024

Related U.S. Application Data
(60) Provisional application No. 63/274,203, filed on Nov. 1, 2021.

(51) Int. Cl. C12N 9/14; A61K 38/00; A61K 47/59; A61P 25/14; C12N 15/113; G01N 33/53

(52) U.S. Cl. CPC C12N 9/14; A61K 47/595; A61P 25/14; C12N 15/113; C12Y 306/01011; G01N 33/5308; A61K 38/00; C12N 2310/14; C12N 2310/531; G01N 2800/2835

(57) ABSTRACT

Aspects of the present disclosure provide methods for treating a neurological disorder using a polyphosphate (polyP)-neutralizing molecule such as a phosphatase or a cationic polymer.

### CLAIM OF PRIORITY

This application claims the benefit of U.S. Provisional Patent Application No. 63/274,203, filed on Nov. 1, 2021, which is incorporated by reference herein in its entirety.

### SEQUENCE LISTING

This application contains a Sequence Listing that has been submitted electronically as an XML file named “07917-0427US1_SL_ST26.XML.” The XML file, created on Sep. 17, 2024 is 6,236 bytes in size. The material in the XML file is hereby incorporated by reference in its entirety.

### FIELD OF THE INVENTION

The subject matter disclosed herein generally relates to methods and compositions for diagnosing, prognosing, and treating neurological disorders.

### BACKGROUND OF THE INVENTION

Polyphosphate (polyP) is a simple, linear (unbranched), inorganic polymer that comprises tens to hundreds of ortho-phosphate (Pi) residues linked by high-energy phosphoanhydride bonds. It is found in every tested cell type in nature, and is conserved across more than a billion years of evolution. Initial studies on bacteria and yeast, which contain high concentrations of cytoplasmic polyP, revealed numerous vital physiological functions for polyP including its role as a source of energy, a Pi reservoir, and a chelator for divalent cations. However, very little is known about the role of polyP in mammalian cells because (i) polyP levels in mammalian cells are low, (ii) mammalian enzymes that synthesize and degrade polyP have not been identified, and (iii) specific and consistent detection and manipulation of polyP in mammalian cells has been technically challenging.

### SUMMARY OF THE INVENTION

The present disclosure is based, at least in part, on the surprising discovery that polyP is released by amyotrophic lateral sclerosis (ALS) and frontotemporal dementia (FTD) astrocytes and kills motoneurons by non-cell autonomous mechanisms involving increased neuronal excitability, increased Ca²⁺ transients, and augmented transcription of genes related to inflammation. It was also found that conditioned medium from primary mouse astrocytes derived from three lines of transgenic ALS mice (SOD1^{G93A} and TDP43^{A314T}) and ALS/FTD (C9ORF72^{HRE}) were toxic to primary ventral spinal cord neurons. Experiments from the van Zundert and Brown laboratories have demonstrated that motoneuron death was prevented by reducing polyP intracellularly by expressing yeast exopolyphosphatase (PPX1) in the astrocytes, and by applying to ALS/FTD-ACM either yeast PPX1 plus pyrophosphatase 1 (PPA1) or nano-sized branched cationic polymers (e.g., UHRA10, G4-PAMAM-NH2) that bind and neutralize polyP. Application of G4-PAMAM-NH₂ or PPX/PPA1 to ACM from iPSC-derived human astrocyte cultures (TDP43^{A90V}) also prevented motoneuron death. Chronic UHRA10 treatment also rescued locomotion deficits and prolonged lifespan in a glial-specific mutant SOD1 Drosophila. It was also shown that treatment of mutSOD1 mice, a mouse model of ALS, with the polyP-neutralizing dendrimer G3-PAMAM-NH₂ or the polyP-neutralizing cationic polymer spermidine prolonged mouse survival.

These findings are immediately relevant to the longstanding observations that non-cell autonomous mechanisms contribute to neurodegenerative diseases, including amyotrophic lateral sclerosis (ALS) and frontotemporal dementia (FTD). Specifically, astrocytes from ALS and ALS-FTD patients and animal models release unidentified neurotoxic factors that kill motoneurons (MNs). Here we assessed the role of inorganic polyphosphate (polyP), a ubiquitous, negatively charged biopolymer recently shown to act as a gliotransmitter. We show higher intracellular polyP staining signals in primary mouse and patient iPSC-derived astrocytes with diverse ALS/FTD-linked mutations (SOD1, TARDBP, C9ORF72). Biochemical quantification assays demonstrate increased polyP levels in astrocyte conditioned media (ACM) from ALS/FTD astrocytes. Studies with familial and sporadic ALS patients reveal that postmortem spinal cord sections display enriched polyP staining signals and that ALS cerebrospinal fluid (CSF) exhibits increased polyP concentrations. We also find that synthetic polyP causes hyperexcitability, increases Ca²⁺ transients and kills MNs. Conversely, MN death is prevented by degrading or neutralizing polyP in ALS/FTD-ACM. Our in vitro results establish excessive astrocyte-derived polyP as a critical factor in non-cell autonomous MN degeneration and a potential therapeutic target for ALS/FTD. The CSF data indicate that polyP might serve as a new biomarker for ALS/FTD.

Accordingly, aspects of the present disclosure provide a method for treating a neurological disorder in a subject, the method comprising administering to a subject in need thereof an effective amount of a polyphosphate (polyP)-neutralizing molecule.

In some embodiments, the polyP-neutralizing molecule is a protein, a nucleic acid encoding a protein, or a polymer.

In some embodiments, the protein is an exopolyphosphatase protein or a polyphosphate binding domain thereof or wherein the protein is an endopolyphosphatase protein or a polyphosphate binding domain thereof. In some embodiments, the exopolyphosphatase protein or the polyphosphate binding domain thereof is derived from yeast or E. coli.

In some embodiments, the exopolyphosphatase protein comprises an amino acid sequence that is at least 85% identical to SEQ ID NO: 1. In some embodiments, the exopolyphosphatase protein comprises an amino acid sequence that is at least 95% identical to SEQ ID NO: 1. In some embodiments, the exopolyphosphatase protein comprises an amino acid sequence that is identical to SEQ ID NO: 1.

In some embodiments, the exopolyphosphatase protein or the polyphosphate binding domain thereof comprises an amino acid sequence that is at least 85% identical to amino acids 306 to 513 of SEQ ID NO: 2. In some embodiments, the exopolyphosphatase protein or the polyphosphate binding domain thereof comprises an amino acid sequence that is at least 95% identical to amino acids 306 to 513 of SEQ ID NO: 2. In some embodiments, the exopolyphosphatase protein or the polyphosphate binding domain thereof comprises an amino acid sequence that is identical to amino acids 306 to 513 of SEQ ID NO: 2. In some embodiments, the exopolyphosphatase protein or the polyphosphate binding domain thereof comprises an amino acid sequence that is at least 85% identical to SEQ ID NO: 2. In some embodiments, the exopolyphosphatase protein or the polyphosphate binding domain thereof comprises an amino acid sequence that is at least 95% identical to SEQ ID NO: 2. In some embodiments, the exopolyphosphatase protein or the polyphosphate binding domain thereof comprises an amino acid sequence that is identical to SEQ ID NO: 2.

In some embodiments, the endopolyphosphatase protein or the polyphosphate binding domain thereof is derived from yeast.

In some embodiments, the endopolyphosphatase protein comprises an amino acid sequence that is at least 85% identical to SEQ ID NO: 3. In some embodiments, the endopolyphosphatase protein comprises an amino acid sequence that is at least 95% identical to SEQ ID NO: 3. In some embodiments, the endopolyphosphatase protein comprises an amino acid sequence that is identical to SEQ ID NO: 3.

In some embodiments, the endopolyphosphatase protein or the polyphosphate binding domain thereof is derived from humans.

In some embodiments, the endopolyphosphatase protein comprises an amino acid sequence that is at least 85% identical to SEQ ID NO: 4. In some embodiments, the endopolyphosphatase protein comprises an amino acid sequence that is at least 95% identical to SEQ ID NO: 4. In some embodiments, the endopolyphosphatase protein comprises an amino acid sequence that is identical to SEQ ID NO: 4.

In some embodiments, the nucleic acid encoding a protein is comprised in a vector. In some embodiments, the vector is a viral vector. In some embodiments, the viral vector is selected from the group consisting of a retroviral vector, a lentiviral vector, an adenoviral vector, an adeno-associated viral vector, a herpes simplex viral vector, and a vaccinia viral vector.

In some embodiments, the polymer comprises a dendrimer, a polyamine, or a combination thereof.

In some embodiments, the dendrimer comprises selected from the group consisting of a polyamidoamine (PAMAM) dendrimer, a polypropylamine (POPAM) dendrimer, a polypropyleneimine (PPI) dendrimer, a polyethylenimine (PEI) dendrimer, a polyarylether (PAE) dendrimer, a polylysine dendrimer, a polyester dendrimer, an iptycene dendrimer, an aliphatic poly(ether) dendrimer, an aromatic polyether dendrimer, an universal heparin reversal agent (UHRA), or a combination thereof.

In some embodiments, the PAMAM dendrimer comprises a generation 3 (G3) PAMAM dendrimer, a generation 4 (G4) PAMAM dendrimer, a generation 5 (G5) PAMAM dendrimer, or a combination thereof.

In some embodiments, the UHRA is selected from the group consisting of UHRA-8, UHRA-9, UHRA-10, and UHRA-14.

In some embodiments, the polyamine comprises wherein the polyamine comprises spermidine, norspermidine, spermine, norspermine, putrescine, cadaverine, polyethylenimine (PEI), polybrene, poly-D-lysine, poly-L-lysine, poly-L-arginine, surfen, protamine, diethylenetriamine (DETA), triethylenetetramine (TETA), or a combination thereof.

In some embodiments, the polyP-neutralizing molecule is formulated in a pharmaceutical composition, which further comprises a pharmaceutically acceptable carrier.

In some embodiments, the subject is a human patient having a neurological disorder. In some embodiments, the neurological disorder is a stroke, a brain injury, neuroinflammation, a neurodegenerative disease, or a combination thereof. In some embodiments, the neurodegenerative disease is amyotrophic lateral sclerosis (ALS), frontotemporal dementia (FTD), ALS and FTD (ALS-FTD), Parkinson's disease (PD), Huntington's disease (HD), or a combination thereof In some embodiments, methods provided herein further comprise administering to the subject an additional therapeutic agent.

Aspects of the present disclosure provide a method for diagnosing a subject as having a neurological disorder, the method comprising: detecting a level of polyphosphate (polyP) in a sample from a subject, and comparing the level of polyP in the sample to a reference level, wherein presence of a level of polyP in the sample that is above the reference level indicates that the subject has a neurological disorder characterized by polyP-induced neurotoxicity.

In some embodiments, the sample comprises cerebrospinal fluid (CSF) or blood.

In some embodiments, methods further comprise administering to a subject in need thereof an effective amount of a polyP-neutralizing molecule. In some embodiments, the polyP-neutralizing molecule is a protein, a nucleic acid encoding a protein, or a polymer.

### DETAILED DESCRIPTION OF THE DRAWINGS

Various aspects and embodiments will be described with reference to the following figures. The figures are not necessarily drawn to scale.

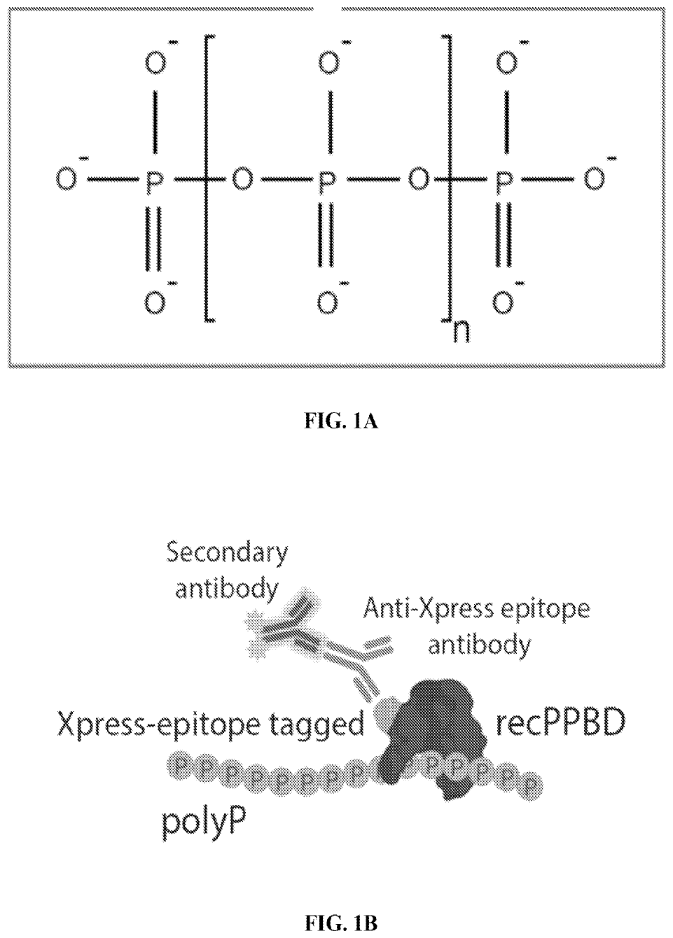

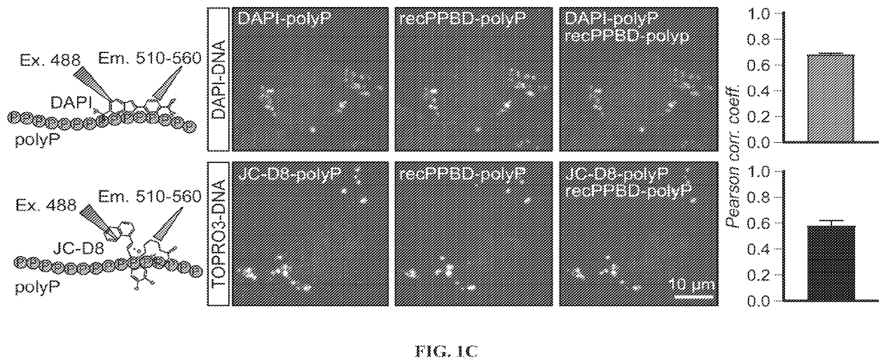

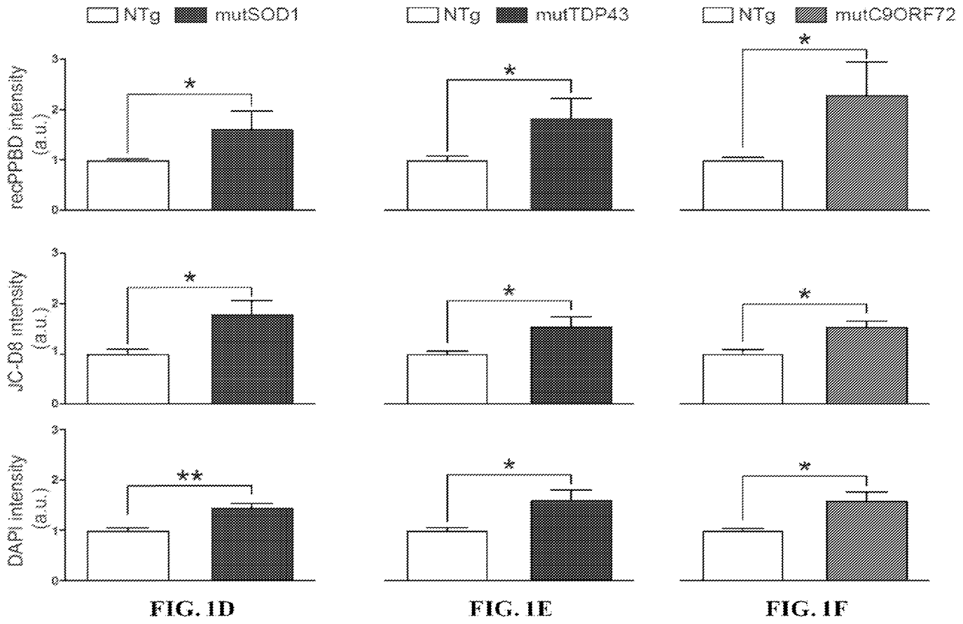

FIGS. 1A-1F include data showing that intracellular polyP level was elevated in primary mutSOD1, mutTDP43 and mutC9ORF72 mouse ALS/FTD astrocytes. FIG. 1A includes a schematic of polyP (n=10-1000). FIGS. 1B-1C includes schematics of the recPPBD immunostaining assay and dyes DAPI-polyP and JC-D8 to detect polyP. Confocal images and Pearson's correlation coefficients showing co-localization (white) of recPPBD (white) with DAPI-polyP (white) or JC-D8 (white) in mutSOD1 astrocytes. Nuclei were detected with DAPI-DNA or TOPRO3. FIG. 1C includes confocal images of mutSOD1, mutTDP43, mutC9ORF72 and non-transgenic littermate (NTg) astrocytes stained with recPPBD and TOPRO3. FIGS. 1D-1F include graphs showing quantification of polyP levels in cytoplasm of mutSOD1 (FIG. 1D), mutTDP43 (FIG. 1E) and mutC9ORF72 (FIG. 1F) astrocytes, versus their NTg astrocytes, determined with recPPBD (upper graphs), JC-D8 (middle graphs) or DAPI-polyP (lower graphs). Graphs show mean±S.E.M. *P<0.05, **P<0.01; unpaired Student's t-test versus NTg.

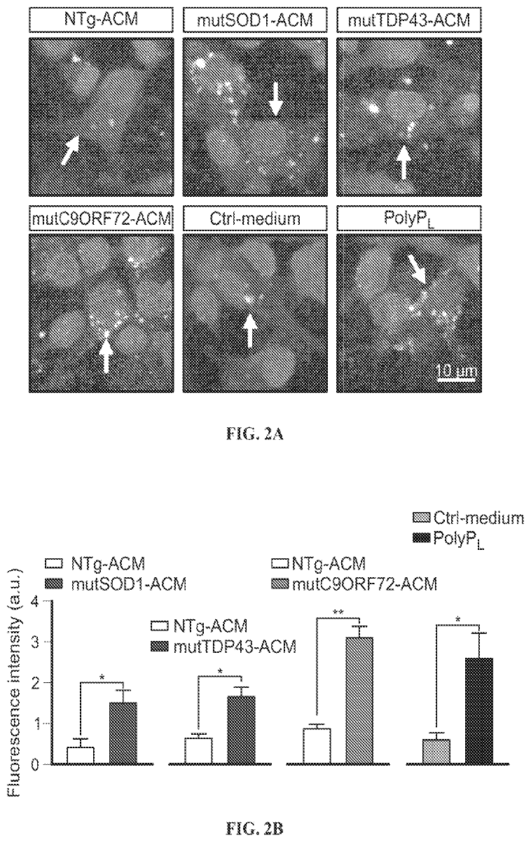

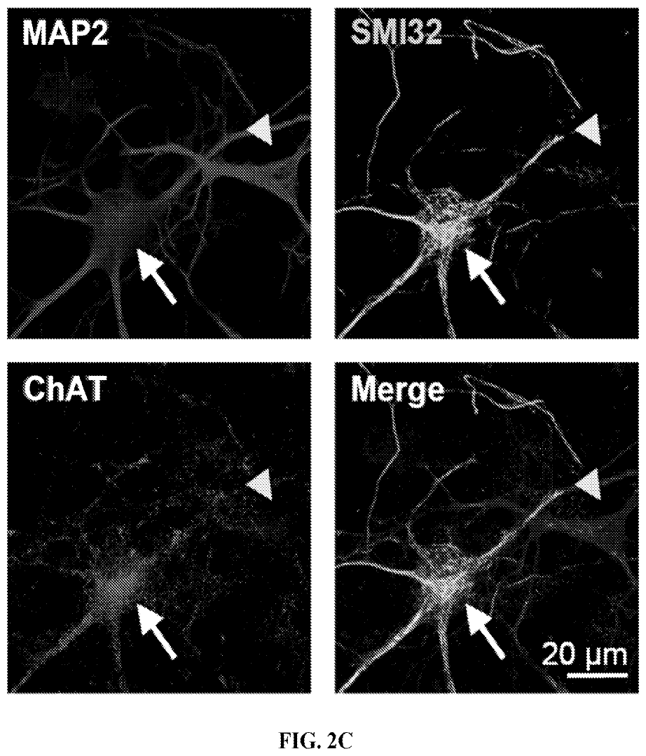

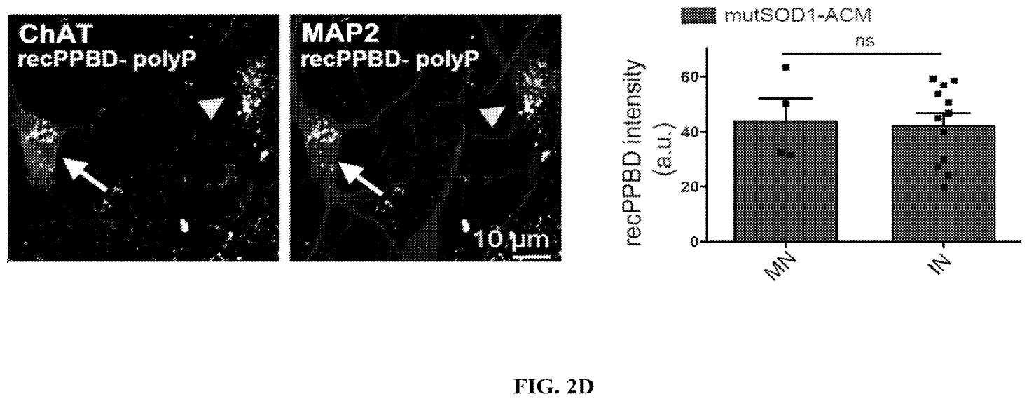

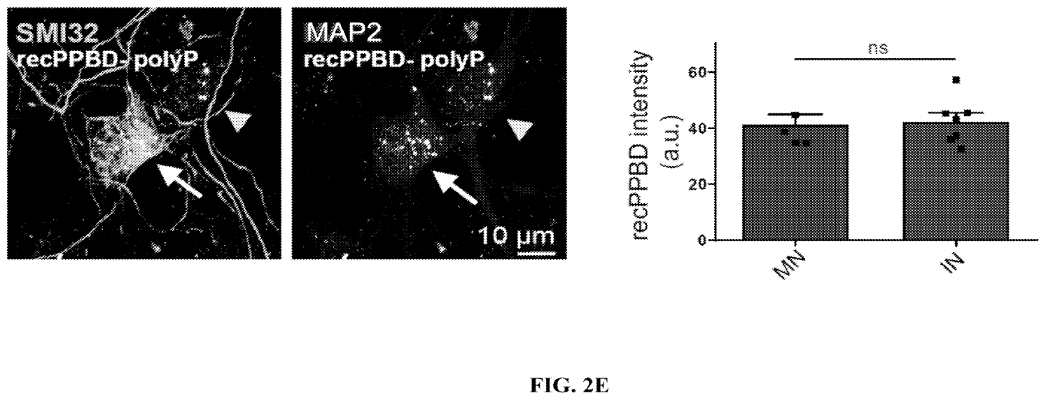

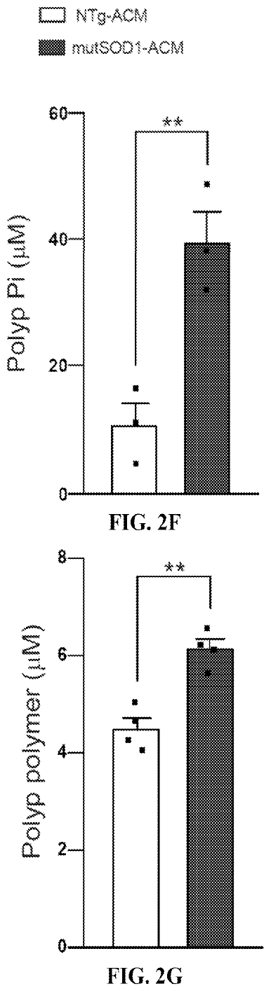

FIGS. 2A-2G include data showing that polyP released by mouse ALS/FTD astrocytes was deposited on neuronal plasma membranes. FIG. 2A includes confocal images of synapsin-tdTomato-expressing spinal cord neurons (gray) stained with JC-D8 (white) showing increased polyP deposition on, or in proximity to, neuronal membranes (arrows: motoneurons) when treated with mutSOD1-ACM, mutTDP43-ACM, mutC9ORF72-ACM or synthetic polyP (50 μM polyPL) versus ACMs from controls (NTg-ACM, Ctrl-medium). FIG. 2B includes graphs showing quantification of JC-D8 fluorescence after applying the ACMs and controls, as indicated. Graphs show mean±S.E.M. *P<0.05, **P<0.01; unpaired Student's t-test versus controls (NTg-ACM or Ctrl-medium) n=3 cultures, analyzing≥6 tdTomato⁺-neurons per condition in each independent experiment). FIG. 2C includes confocal images showing immunofluorescence for MAP2 (gray), SMI32 (gray) and ChAT (gray) in ventral spinal cord cultures. All 3 neuronal markers co-localize (white arrows); while ChAT and SMI32 can be used to identify MNs, MAP2 can detect INs (white/gray arrowheads). FIGS. 2D-2E include confocal images for the polyP probe recPPBD (white), for the IN marker MAP2 (gray), and for the MN markers ChAT (gray) (FIG. 2D) or SMI32 (gray) (FIG. 2E). Images and quantification show an equal deposition of polyP on, or near, membranes of MNs (ChAT⁺ or SMI32⁺; white arrows) and INs (MAP2⁺ only; white/gray arrowheads) when treated with mutSOD1-ACM (mean±S.E.M. *P>0.05 (not significant, ns); unpaired Student's t-test MN versus IN). FIGS. 2F-2G include graphs showing P/C-extracted polyP from NTg-ACM or mutSOD1-ACM, either digested by recPPX1/PPase1 and polyP Pi levels colorimetrical quantified by malachite (FIG. 2F), or not digested to measure polyP polymer levels fluorometrical by JC-D8 (FIG. 2G) (mean±S.E.M. **P<0.01; unpaired Student's t-test; n=3-4 independent ACM analyzed in duplicate).

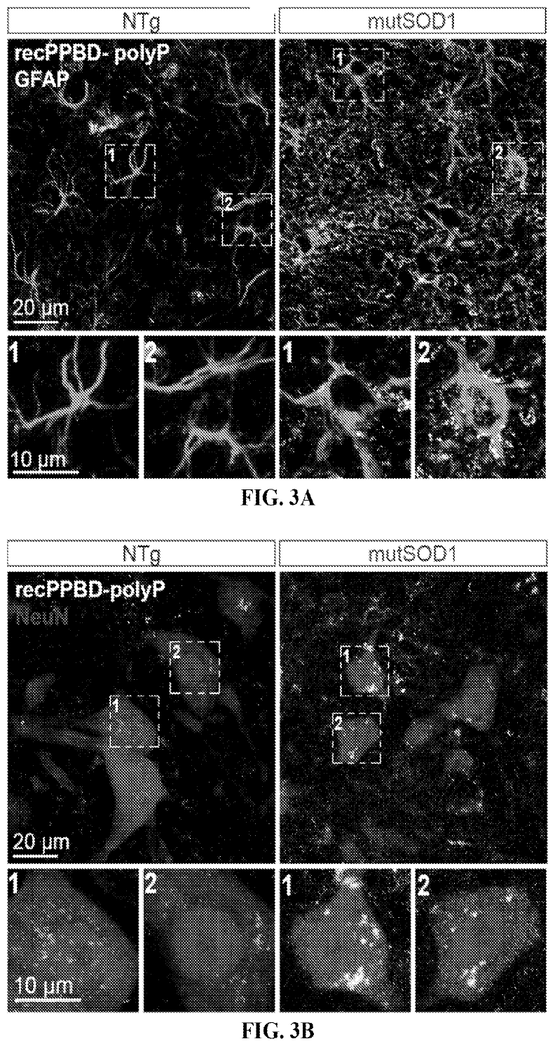

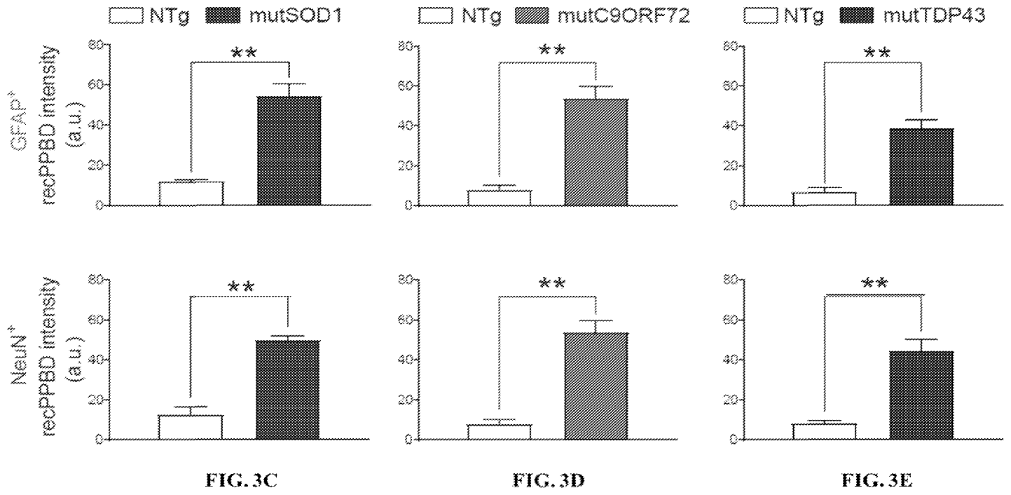

FIGS. 3A-3E include data showing that polyP was enriched in spinal cord sections of ALS/FTD mouse models. FIGS. 3A-3B include confocal images for recPPBD (white) with astrocyte (GFAP) (FIG. 3A) or neuronal (NeuN) (FIG. 3B) markers (gray) in ventral lumbar spinal cords of mutSOD1 and NTg mice. Insets: high-intensity recPPBD-immunoreactive puncta in GFAP and NeuN-positive cells in mutSOD1 sections. FIGS. 3C-3E include graphs showing quantification of recPPBD fluorescence in cytoplasm of GFAP (FIGS. 3C-3E, top) and NeuN-positive cells (FIGS. 3C-3E, bottom) in sections from mutSOD1 (FIG. 3C), mutC9ORF72 (FIG. 3D), and mutTDP43 (FIG. 3E) mice and each corresponding NTg. Graphs show mean±S.E.M. **P<0.01; unpaired Student's t-test versus NTg.

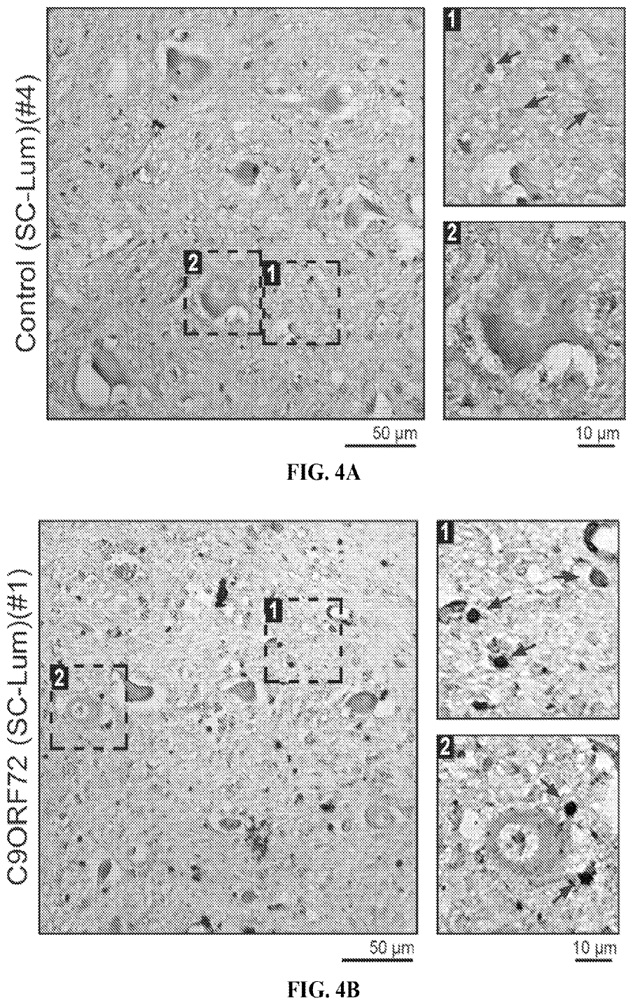

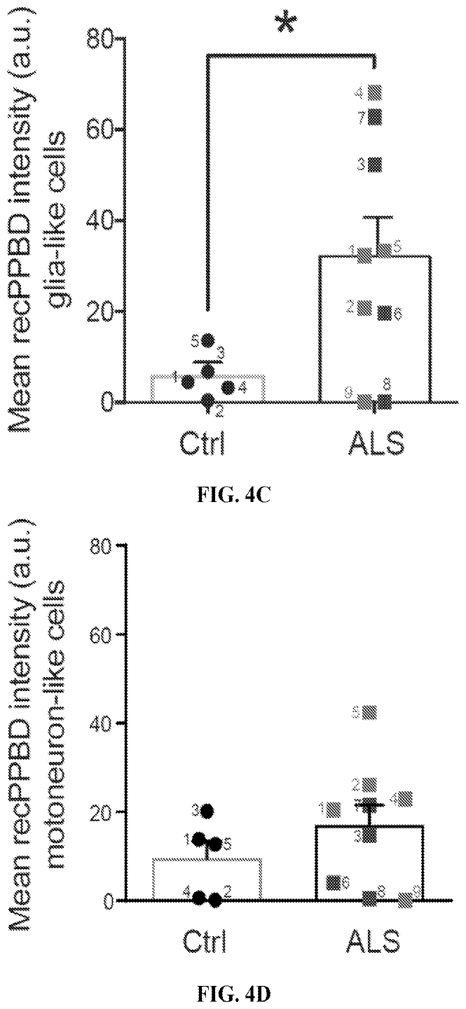

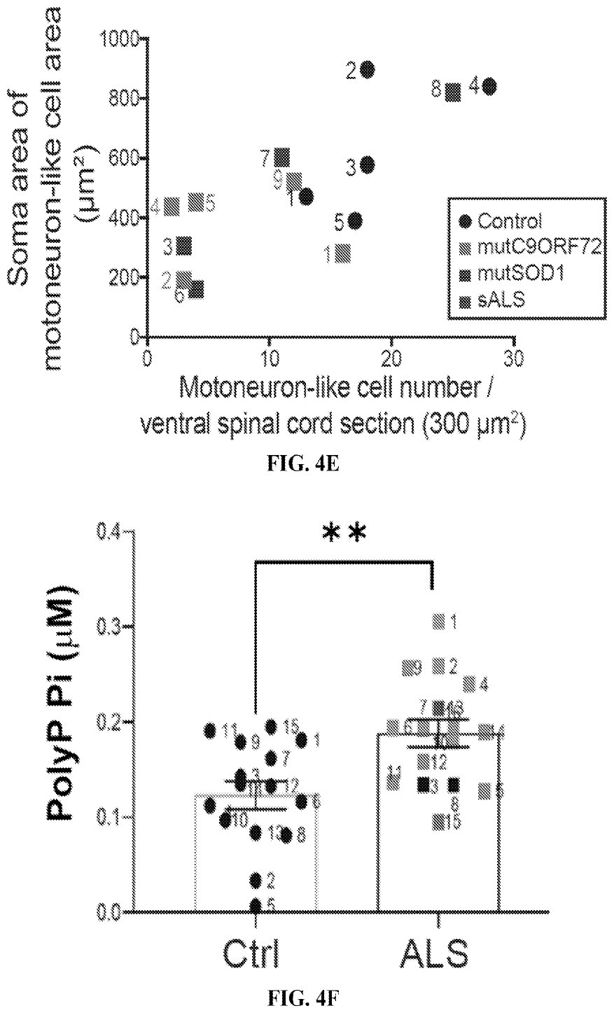

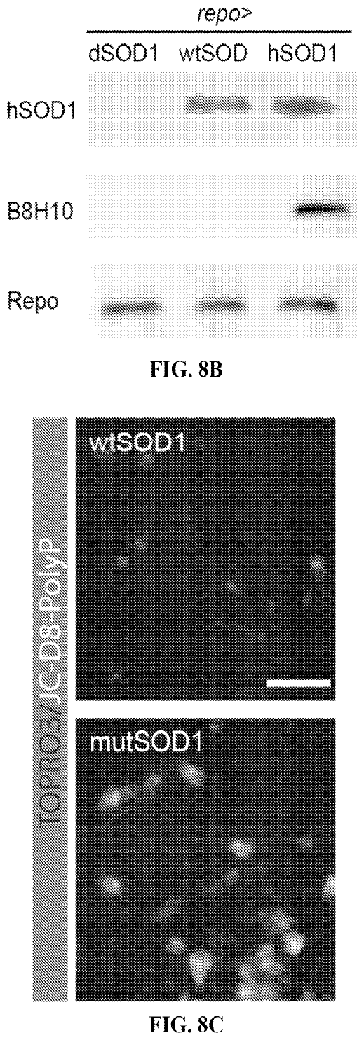

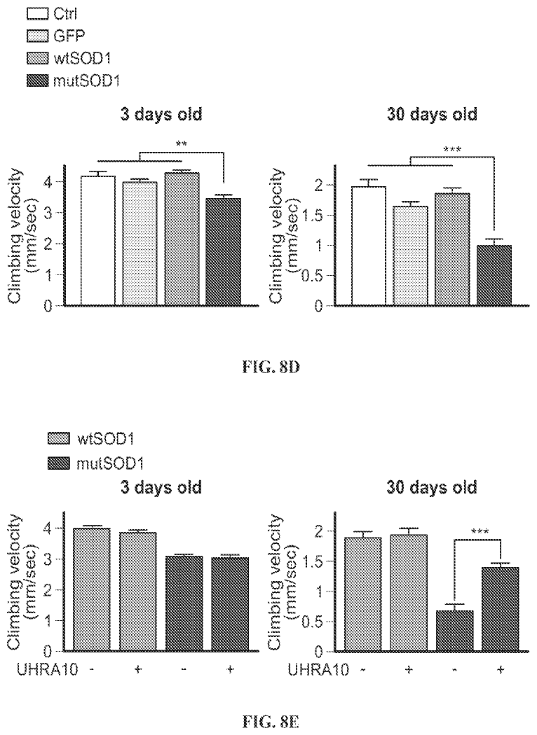

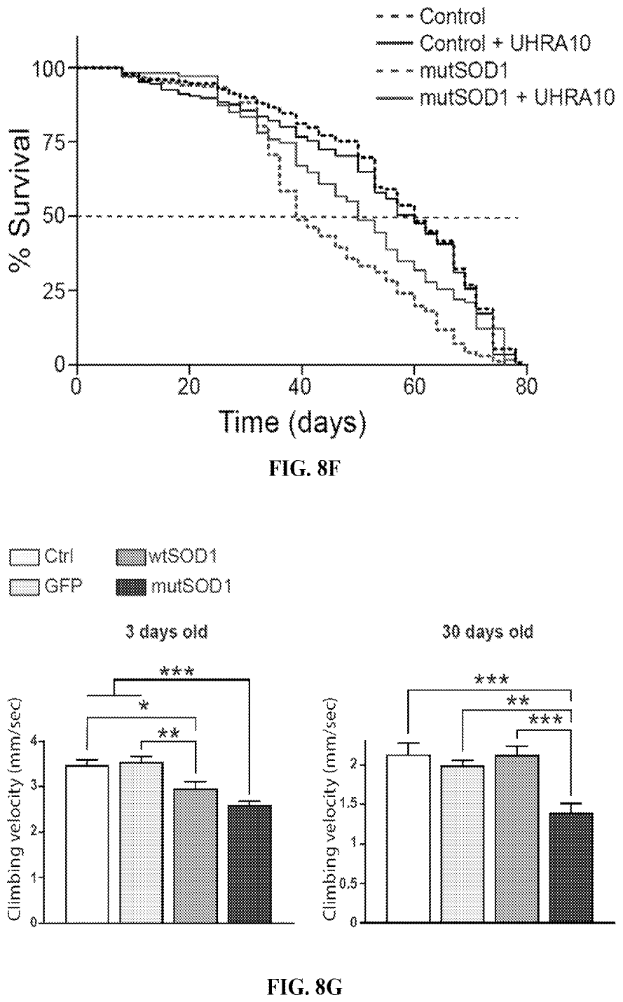

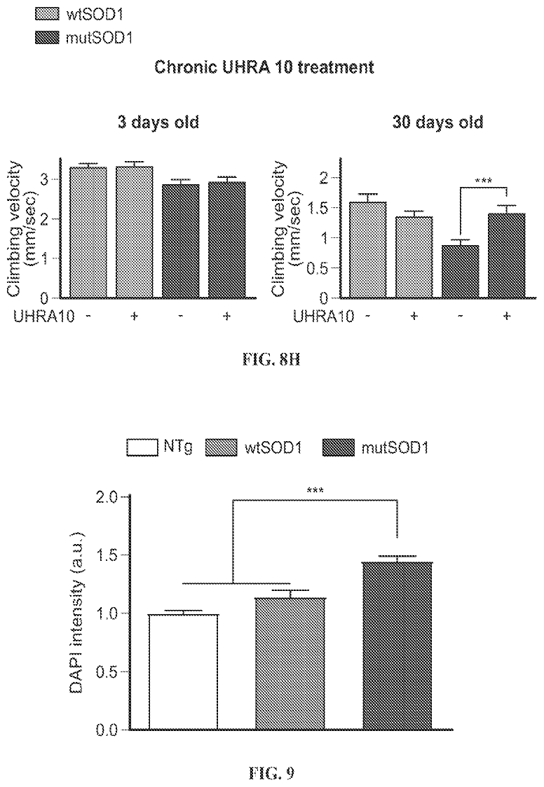

FIGS. 4A-4F includes data showing polyP level is higher in postmortem spinal cord tissues from fALS and sALS patients. FIGS. 4A-4B include representative immunohistochemistry micrographs for recPPBD in spinal cords from control (FIG. 8A) and ALS (C9ORF72) (FIG. 8B) subjects. In ALS, magnifications show recPPBD-immunoreactivity in glial-like cells (box #1, red arrows) and smaller MN-like cells (box #2). FIGS. 4C-4D include graphs of quantification of recPPBD-immunoreactivity in cytoplasm of glial-like (FIG. 8C) and MN-like (FIG. 8D) cells from control (n=5) and ALS (n=9) tissues. Graphs show mean±S.E.M. *P<0.05; Mann-Whitney U-test versus control (≥10 MN-like cell and 25 glia-like cells were analyzed per subject). FIG. 4E includes a graph of soma area (y-axis) versus number (x-axis) of MN-like cells. FIG. 4F includes a graph of silica extracted polyP from the CSF of healthy control subjects (Ctrl, n=15) and ALS patients (n=16) and colorimetrical quantified as Pi by malachite (mean±S.E.M. **P<0.01; unpaired two-tailed Student's t-test).

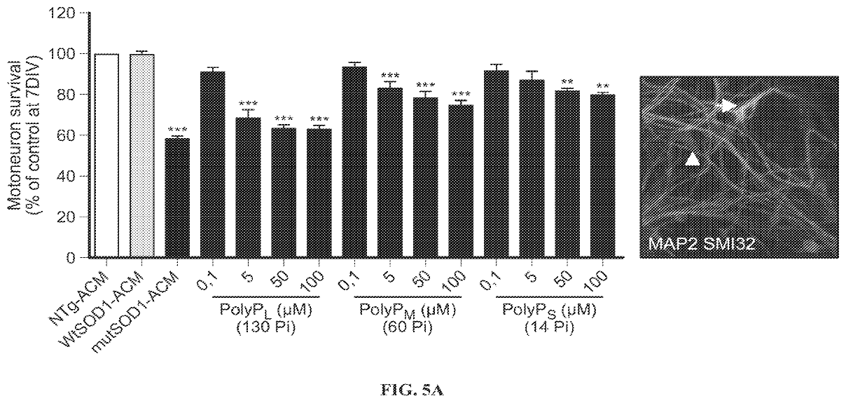

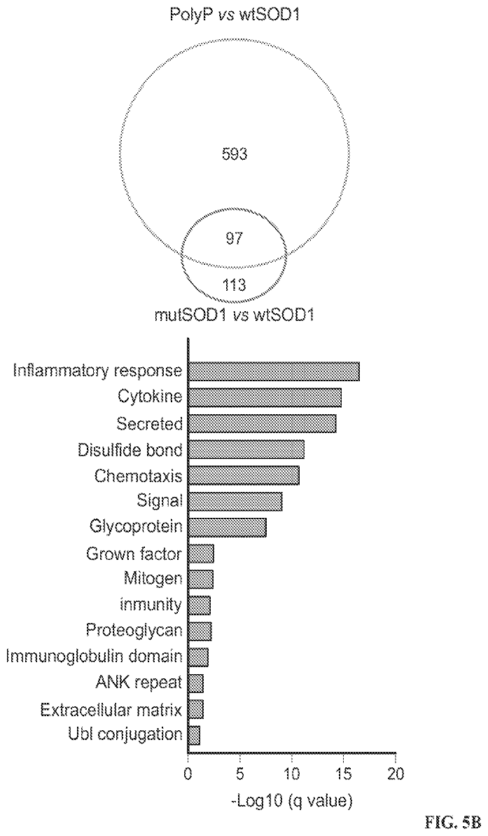

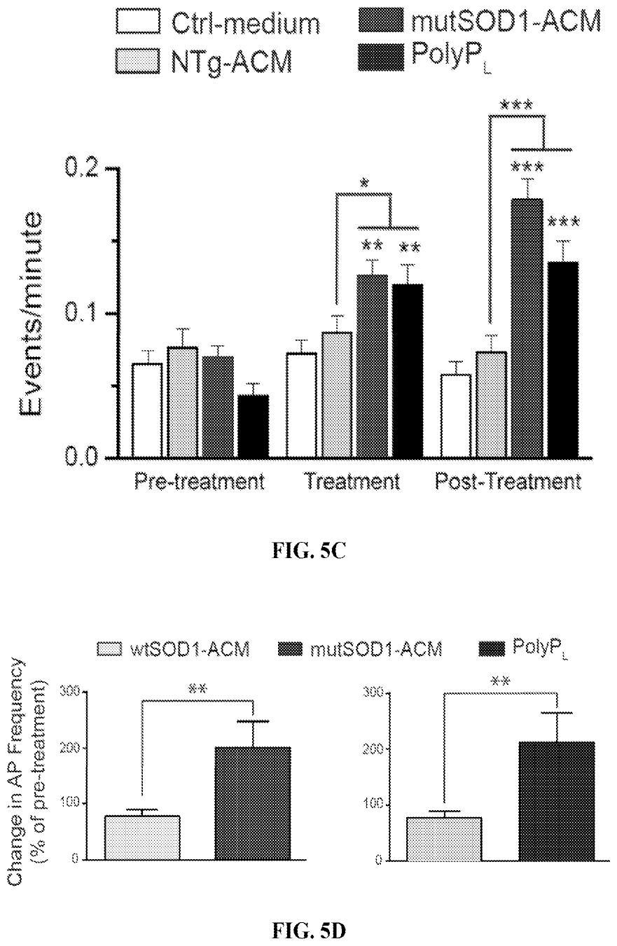

FIGS. 5A-5D include data showing that application of synthetic polyP to wild-type spinal cord neurons reproduced the toxicity effect of mutSOD1-ACM. FIG. 5A includes data showing fewer motoneurons (SMI32 plus MAP2 immunostaining) found in 7 DIV spinal cord cultures treated (4 days) with mutSOD1-ACM or synthetic polyP versus controls (NTg-ACM and wtSOD1-ACM) as indicated. FIG. 5B includes a Venn diagram and functional annotation analysis of the 97 commonly expressed transcripts in spinal cord cultures: polyPL vs. wtSOD1-ACM and mutSOD1-ACM vs. wtSOD1-ACM. FIGS. 5C-5D include graphs showing that polyPL (100 μM) and mutSOD1-ACM increase intracellular Ca²⁺ transients and spontaneous action potential firing, respectively, versus controls. KCl (30 mM) induces calcium activity in all cells. Graphs show mean±S.E.M. *P<0.05, **P<0.01, and ***P<0.001; one-way ANOVA (FIG. 5A, FIG. 5C) and paired Student's t-test (FIG. 5D) versus controls (NTg-ACM, Ctr-medium or wtSOD1-ACM).

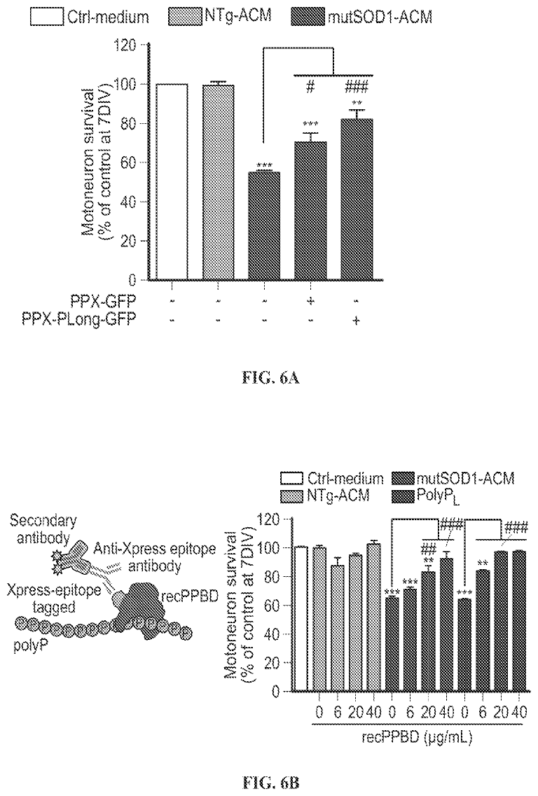

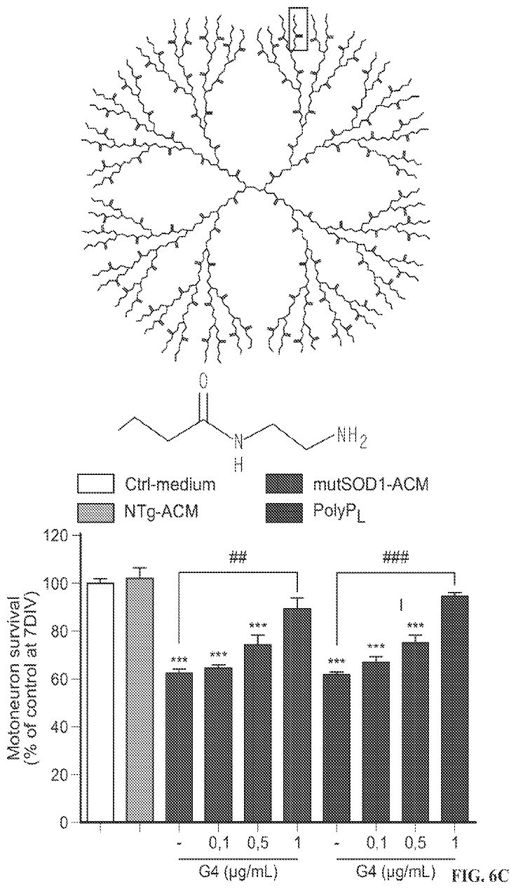

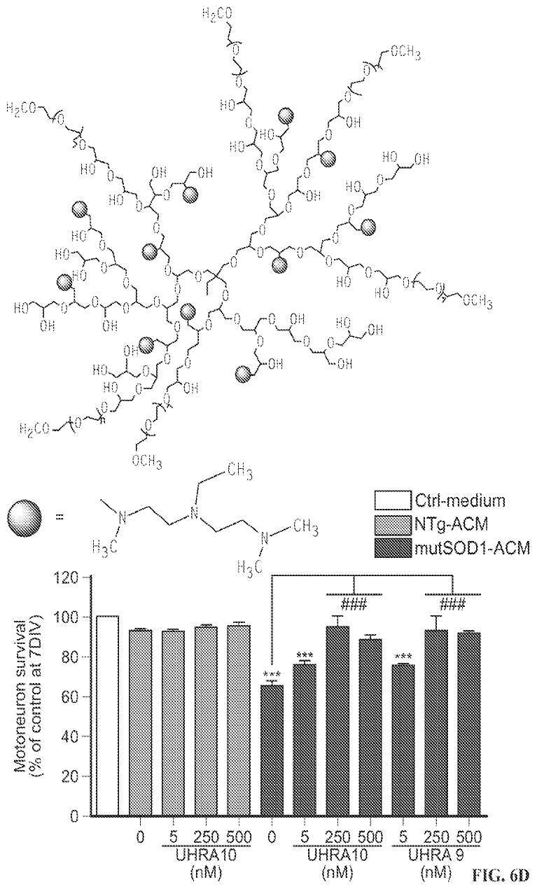

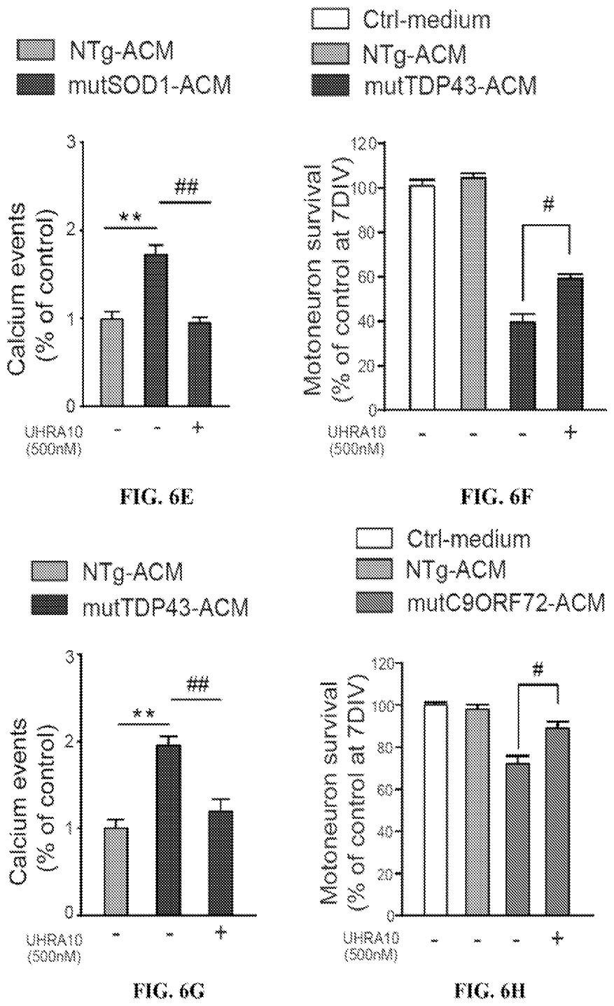

FIGS. 6A-6J include data showing that reducing polyP in ALS astrocytes, or neutralizing polyP in ALS/FTD-ACM, restored intracellular Ca²⁺ transients and prevents motoneuron death. FIG. 6A includes a graph showing that expression of PPX1 in mutSOD1 astrocytes (using AAV9-PPX-GFP or AAV9-PPX-PLong-GFP) lowered toxicity of mutSOD1-ACM to motoneurons. Treatment with recPPBD (FIG. 6B), G4-PAMAM-NH2 (FIG. 6C), or UHRA9/10 (FIG. 6D) rescued motoneurons from mutSOD1-ACM and polyP_{L} (100 μM). Schematics of the different polyP sequestering molecules are depicted. Application of UHRA10 to mutSOD1-ACM (FIG. 6E), mutTDP43-ACM (FIGS. 6F-6G) or mutC9ORF72-ACM (FIGS. 6H-6I) reduced motoneuron death (FIG. 6F, FIG. 6H) and Ca²⁺ transients (FIG. 6E, FIG. 6G, FIG. 6I). Graphs show means±S.E.M. **P<0.01, and ***P<0.001; one-way ANOVA versus controls (NTg-ACM or Ctrl-medium). #P<0.05, ##P<0.01, and ###P<0.001; one-way ANOVA versus mutSOD1-ACM or polyPL. FIG. 6J includes a graph showing treatment with the polyP degrading enzymes recPPX/PPase or CIP.

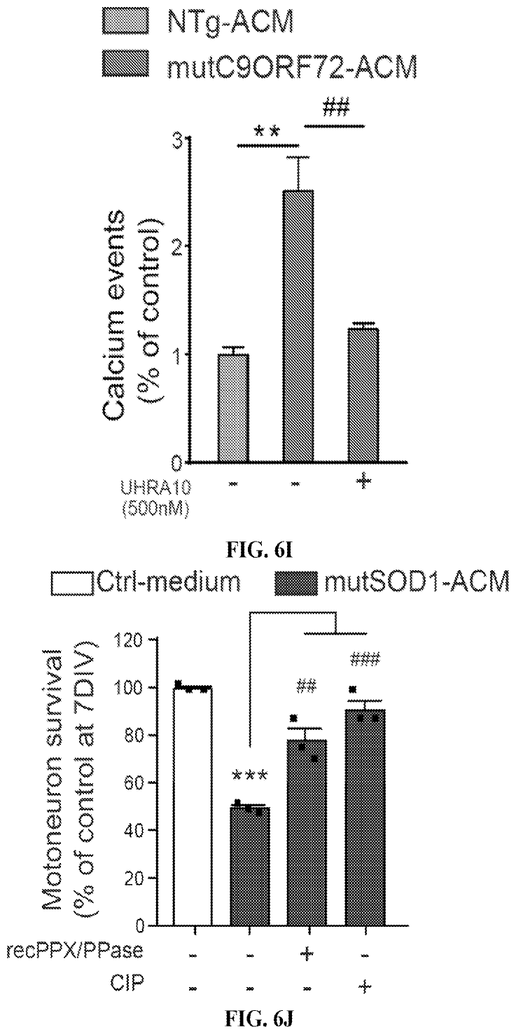

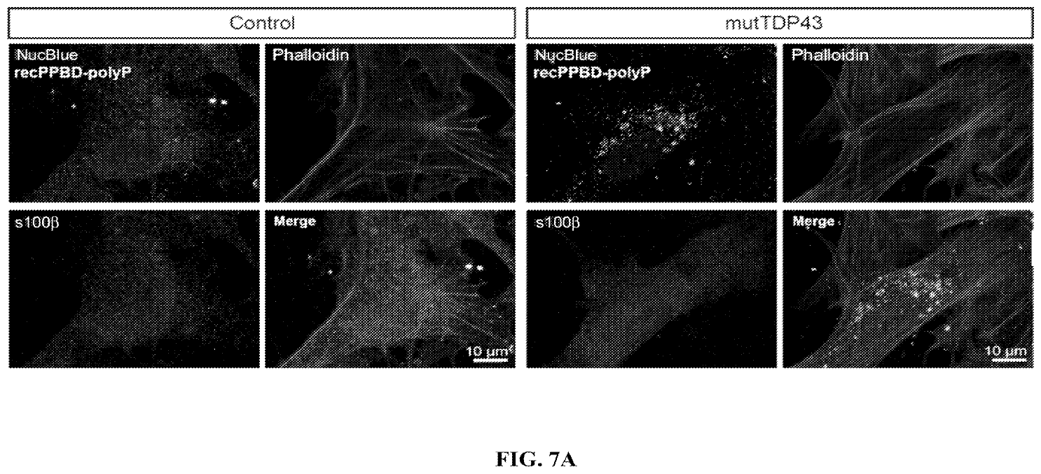

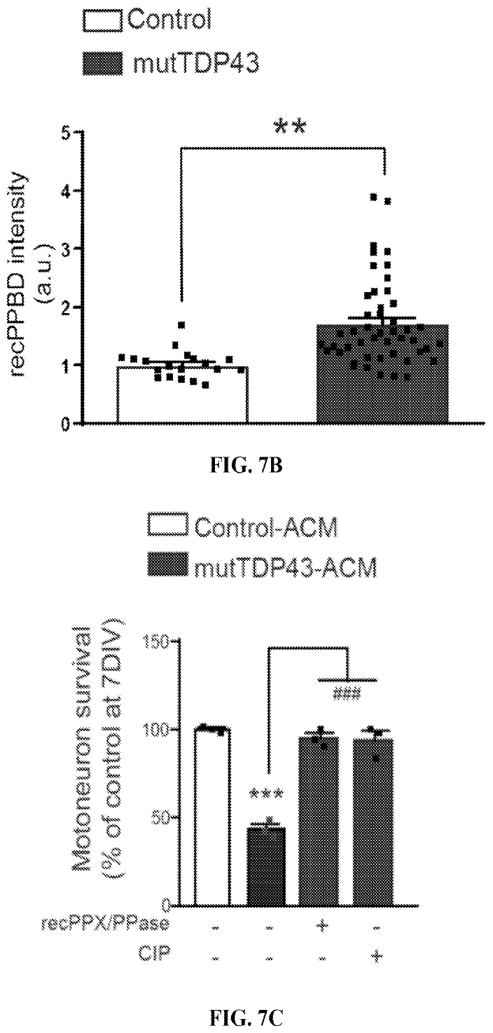

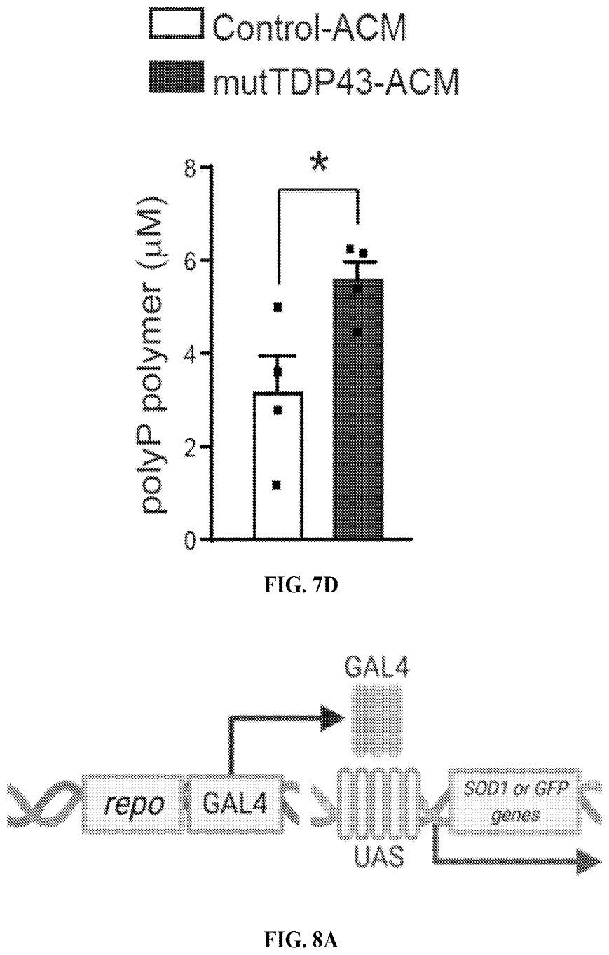

FIGS. 7A-7D include data showing that PolyP levels are elevated in mutTDP43 patient iPSC-derived astrocytes and degrading polyP in mutTDP43-ACM prevents MN death. FIG. 7A includes confocal images for recPPBD (white), Nucblue (gray), phalloidin (gray) and mature astrocyte marker s100β (gray). Mature astrocytes were generated from iPSCs derived from an ALS/FTD TDP43A90V patient (mutTDP43) and a healthy control subject (control). FIG. 7B includes a graph of quantification of cytoplasmic polyP levels of individual human control and mutTDP43 astrocytes determined with recPPBD (mean±S.E.M. ***P<0.001; unpaired Student's t-test mutTDP43 versus control; ≥20 cells/condition from 2 independent differentiations). FIG. 7C includes a graph of treatment with of mutTDP43-ACM with the polyP degrading enzymes recPPX/PPase or CIP rescues MNs from death (means±S.E.M. ***P<0.001; one-way ANOVA versus controls control-ACM. ###P<0.001; one-way ANOVA versus mutTDP43-ACM (n=3 cultures, analyzing≥12 randomly fields (≥400 cells) per condition in each independent experiment). FIG. 7D includes a graph of P/C-extracted polyP polymer from control-ACM and mutTDP43-ACM fluorometrical quantified by JC-D8 (mean±S.E.M. *P<0.05; unpaired Student's t-test; n=4 from two independent differentiations).

FIGS. 8A-8F include data showing that UHRA10 polyP neutralizing molecule rescued the ALS phenotype of a Drosophila glial mutSOD1 expressing model. FIG. 8A includes a schematic depiction of the UAS-GAL4 system for glia specific expression of human SOD1 genes in flies under the repoGal4 promoter: repo>UAS-mutSOD1^{A4V} (mutSOD1) flies were used as ALS model. As controls, repo>UAS-wtSOD1 (wtSOD1), repoGal4>+(Ctrl), repo>UAS-GFP (GFP), and repo>UAS-dSOD1 (Drosophila SOD1, dSOD1) were used. FIG. 8B includes an image of protein extract of 30 days old flies (heads) submitted to western blot for hSOD1 and B8H10 to detect native and misfolded human SOD1, respectively. FIG. 8C includes confocal images for JC-D8 (white) and TOPRO (black) in brains (optic lobe) of wtSOD1 and mutSOD1 flies. FIG. 8D includes graphs of climbing test using 3 and 30 days old mutSOD1 and control flies. FIG. 8E includes graphs of climbing test using 3 and 30 days old mutSOD1 and wtSOD1 flies in the absence and presence of UHRA10 as indicated: flies were fed 500 nM UHRA10 (UHRA10) or vehicle (H₂O, not indicated) every other day from day 1 post-eclosion. Data are shown only for female flies. FIG. 8F shows survival curves of mutSOD1 and wtSOD1 flies in the presence and absence UHRA10. Graphs show mean±S.E.M. **P<0.01; ***P<0.001; One-way ANOVA. FIG. 8G includes graphs of climbing test using 3 and 30 days old male mutSOD1 and male control flies. FIG. 8H includes graphs of climbing test using 3 and 30 days old male mutSOD1 and male wtSOD1 flies in the absence and presence of UHRA10 as indicated: flies were fed 500 nM. Graphs in FIGS. 8G-8H show mean±S.E.M. *P<0.05; **P<0.01; ***P<0.001; One-way ANOVA.

FIG. 9 includes a graph showing quantification of cytoplasmic polyP levels (relative a.u.) with the use of DAPI-polyP in astrocytes carrying mutSOD1 relative to both astrocytes derived from nontransgenic littermates (NTg) and those expressing human wild-type SOD1 (wtSOD1). n=3-4 independent experiments. Graph shows means±S.E.M. *P<0.05; one-way ANOVA compared to Ctrl and wtSOD1.

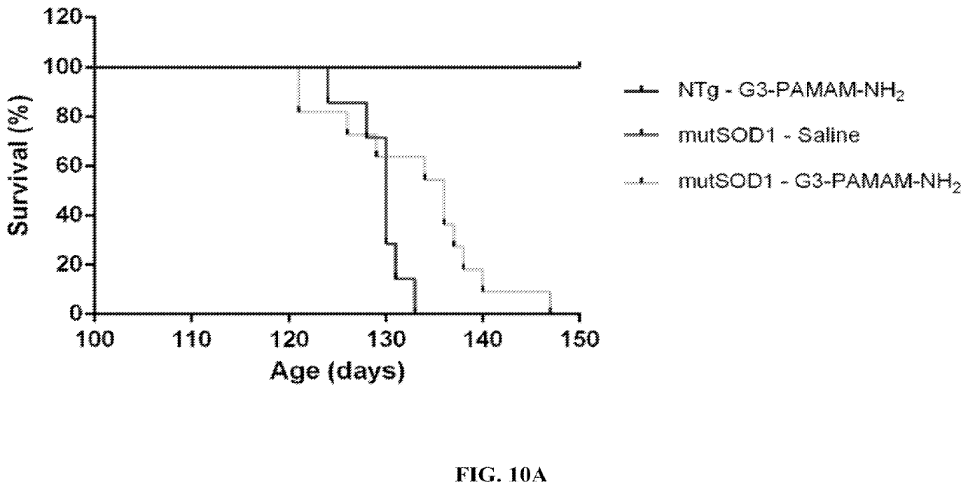

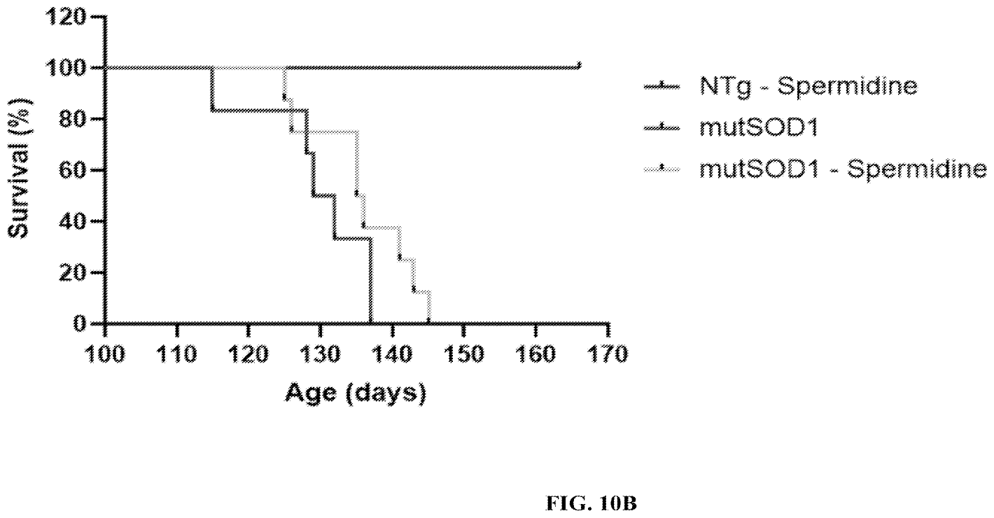

FIGS. 10A-10B include graphs showing prolonged survival of mutSOD1 mice treated with a polyP neutralizing dendrimer (G3-PAMAM-NH₂; FIG. 10A) or with a polyP neutralizing cationic polyamine (spermidine; FIG. 10B).

The details of one or more embodiments of the invention are set forth in the description below. Other features or advantages of the present invention will be apparent from the following drawings and detailed description of several embodiments, and also from the appended claims.

### DETAILED DESCRIPTION

The present disclosure is based, at least in part, on the finding that polyP is released from amyotrophic lateral sclerosis and frontotemporal dementia (ALS/FTD) astrocytes and causes hyperexcitability, increases Ca²⁺ transients, and kills motoneurons. It was also demonstrated that motoneuron death was prevented by reducing polyP in ALS/FTD astrocytes by expressing yeast polyphosphatase (PPX1) alone or in combination with pyrophosphatase 1 (PPA1) or by applying nano-sized branched cationic polymers such as UHRA-10 or G4-PAMAM-NH₂.

Astrocytes play a critical role in the maintenance of the health and function of neurons. Loss-of-function as well as gain-of-toxic-function of astrocytes have been implicated in various neurodegenerative disorders, including amyotrophic lateral sclerosis (ALS), frontotemporal dementia (FTD), C9ORF72-mediated ALS-FTD (C9ALS-FTD), Alzheimer's disease (AD), Parkinson's disease (PD) and Huntington's disease (HD) (di Domenico et al., 2019; Hallmann et al., 2017; Orellana et al., 2011; Phatnani and Maniatis, 2015). For ALS and C9ALS-FTD (together termed ALS/FTD), there is compelling evidence that astrocytic non-cell autonomous toxicity is a major cause of motoneuron cell death, which involves the release of soluble factor(s) by mouse and human astrocytes harboring disease-causing mutant genes in the absence of microglia (Birger et al., 2019; Fritz et al., 2013; Haidet-Phillips et al., 2011; Ikiz et al., 2015; Jury et al., 2020; Nagai et al., 2007; Re et al., 2014; Rojas et al., 2014; Varcianna et al., 2019). A1 astrocytes, induced by specific cytokines secreted by neuroinflammatory microglia (IL-1α, TNFα, and C1q), may also induce non-cell autonomous neurodegeneration as these reactive astrocytes are observed in post-mortem CNS tissue of patients with several neurodegenerative diseases, including ALS (Guttenplan et al., 2020; Liddelow et al., 2017). So far, the identity of the neurotoxic factors released by ALS/FTD and A1 astrocytes has not been established.

ALS and FTD form a continuous spectrum of aggressive neurodegenerative diseases, affecting primarily motoneurons and fronto-temporal neurons, respectively (Ling et al., 2013). The majority of ALS patients have the sporadic form of the disease (sALS), but about 10% of the cases have familial ALS (fALS), which is associated with pathogenic mutations in genes such as superoxide dismutase 1 (hereafter “mutSOD1”), transactive response DNA-binding protein 43 (TARDBP encoding TDP-43; “mutTDP43”), and C9ORF72 (“mutC9ORF72”) (Taylor et al., 2016; Renton et al., 2014), which is characterized by an intronic hexanucleotide expansion. Patients carrying mutC9ORF72 can suffer from FTD, ALS or simultaneously both diseases, and referred to as C9ALS-FTD, and thus exhibiting motor dysfunction as well as cognitive impairment (Ling et al., 2013). Independently of genotype, sporadic and familial ALS and C9ALS-FTD patients share many clinical and histopathological features, an observation that suggests that different subsets of ALS/FTD share a convergent, common mechanistic pathway. Elucidating the common elements in ALS/FTD is thus an important objective en route to developing widely applicable and effective diagnostic and treatment options for these devastating diseases.

Increasing evidence from animal models implicates astrocytes in the pathology of ALS/FTD (Clement et al., 2003; Lepore et al., 2008; Papadeas et al., 2011; Qian et al., 2017; Tong et al., 2013; Wang et al., 2011; Yamanaka et al., 2008). Astrocytic non-cell autonomous processes have been demonstrated in many studies using co-cultures of control (healthy wild-type) motoneurons together with mouse or human astrocytes harboring disease-causing mutant genes (Phatnani and Maniatis, 2015). Moreover, using astrocyte-conditioned media (ACM) from mutated astrocytes, it has been firmly demonstrated that ALS/FTD astrocytes release soluble factors that induce non-cell autonomous toxicity in motoneurons. Thus, ACM from mouse and human astrocytes with known (SOD1, TARDBP, C9ORF72 genes) (Birger et al., 2019; Fritz et al., 2013; Haidet-Phillips et al., 2011; Ikiz et al., 2015; Jury et al., 2020; Nagai et al., 2007; Rojas et al., 2014; Varcianna et al., 2019) and unknown causes (sporadic cases) (Haidet-Phillips et al., 2011; Re et al., 2014) have been shown to kill motoneurons effectively in vitro. Chronic infusion of ACM from mutSOD1 astrocytes also has led to spinal motoneuron degeneration and neuromuscular dysfunction in healthy rats (Ramirez-Jarquin et al., 2017). Mechanistically, ALS ACM triggers motoneuron death by inducing hyperexcitability that leads to Ca²⁺ overload and oxidative stress (Fritz et al., 2013; Rojas et al., 2014). Motoneuron hyperexcitability is an early and critical feature in ALS models (Devlin et al., 2015; Wainger et al., 2014; van Zundert et al., 2008, 2012) and patients (Geevasinga et al., 2016), and is mediated by increased activation of voltage-sensitive sodium (Naᵥ) channels and/or inactivation of voltage-sensitive potassium (Ky) channels. While the generation of ALS/FTD-ACM has increased the potential for identifying the toxic factors that kill motoneurons, standard biochemical approaches searching for the presence of candidate small organic molecules, such as neuro- and gliotransmitters and cytokines/chemokines (e.g., glutamate, ATP, and TNFα) (Nagai et al., 2007; Re et al., 2014) or of peptides/proteins in unbiased manner by mass spectrometry (Mishra et al., 2021; our unpublished data), thus far have been futile.

Hence, there is a necessity to further expand understanding the range of molecules secreted by ALS/FTD astrocytes that may mediate non-cell autonomous toxicity to motoneurons. Studies described herein demonstrate the role of astrocyte-secreted polyphosphate (polyP) as an adverse pathogenic molecule in ALS.

Described herein are studies that utilized several independent experimental approaches to demonstrate systematically in vitro and in vivo that levels of inorganic polyP were elevated in mouse astrocytes with mutations in SOD1, TARDBP or C9ORF72, three genes whose mutations encompass the majority of familial ALS/FTD patients. In post-mortem spinal cord tissue, polyP was also enriched in astrocytes from fALS and sALS patients. These findings collectively indicate that polyP enrichment in spinal cord astrocytes is a widely present hallmark of ALS/FTD. The analysis described herein further revealed that the level of polyP was increased in the ACM from ALS/FTD astrocytes with three different genotypes—mouse and human—quantification biochemically. Importantly, by using five distinct approaches to reduce or sequester intracellular and extracellular polyP, respectively, it was demonstrated that motoneurons could be rescued from the toxicity induced by the media conditioned by ALS/FTD astrocytes. Conversely, exposure of wild-type spinal cord neurons to polyP reproduced the toxic effects of ALS-ACM, causing increased neuronal excitability, increased Ca²⁺ transients, enhanced motoneuron cell death and augmented transcription of genes related to inflammation. These results indicate that polyP released from ALS/FTD astrocytes is both necessary and sufficient to cause non-cell autonomous toxicity to neurons. These results also suggest that polyP can contribute to astrocyte-initiated neuronal injury and death in other neurodegenerative diseases, including in Parkinson's disease (PD) and Huntington's disease (HD), possibly in combination with other astrocyte-derived substance (e.g., glutamate, ATP, u-synuclein) as well as in other types of brain pathology including stroke, head trauma, and brain injury. inflammation

Accordingly, the present disclosure provides, in some aspects, therapeutic uses of polyP-neutralizing molecules for treating a neurological disorder such as a stroke, a brain injury, neuroinflammation, a neurodegenerative disease (e.g., ALS, FTD, ALS/FTD, PD, and HD), or a combination thereof.

I. polyP-Neutralizing Molecules and Compositions Comprising Such

Aspects of the present disclosure provide polyP-neutralizing molecules such as polyP-neutralizing proteins, nucleic acids encoding such proteins, and polymers such as polyamines and dendrimers.

The term “polyP-neutralizing molecule,” as used herein, refers to a molecule (e.g., a small molecule, a polymer, or a protein) that neutralizes or counteracts (including significantly) the activity and/or effect (e.g., polyP-mediated neurotoxicity) of polyP in vitro, in situ, and/or in vivo. The term “neutralizing” implies no specific mechanism of biological action whatsoever, and expressly includes and encompasses all possible pharmacological, physiological, and biochemical interactions with polyP whether direct or indirect, and whether interacting with polyP, its binding partner, or through another mechanism, and its consequences which can be achieved by a variety of different, and chemically divergent, compositions.

In some examples, the polyP-neutralizing molecule alters the level of polyP (e.g., reduces the level of polyP), thereby neutralizing or counteracting the activity and/or effect (e.g., polyP-mediated neurotoxicity). In some examples, the polyP-neutralizing molecule reduces the level of polyP by hydrolyzing and/or degrading polyP polymers into smaller polymers and/or orthophosphate (Pi) residues.

Non-limiting examples of a polyP-neutralizing molecule for use in the methods described herein for treating a neurological disorder include a polyP-neutralizing protein (e.g., a polyphosphatase or polyphosphate binding domain thereof), nucleic acids encoding a polyP-neutralizing protein, a polymer (e.g., a polyamine such as spermidine or a dendrimer such as a PAMAM dendrimer or a UHRA), polyP-neutralizing small molecules, polyP-neutralizing peptides or nucleic acids encoding such, and polyP-neutralizing antibodies.

(a) polyP-Neutralizing Proteins

Any protein suitable for neutralizing polyP (e.g., neutralizing polyP-mediated neurotoxicity) can be used in methods for treating a neurological disorder as disclosed herein. A polyP-neutralizing protein can bind to polyP and/or to a binding partner of polyP and/or degrade polyP. As such, the polyP-neutralizing protein can be catalytically active or inactive.

In some embodiments, a polyP-neutralizing protein used in the methods described herein neutralizes a polyP biological activity and/or a polyP biological effect by at least 10% (e.g., at least 20%, at least 30%, at least 40%, at least 50%, at least 60%, at least 70%, at least 80%, at least 90%, or more). In some embodiments, a polyP-neutralizing protein used in the methods described herein alters the level of polyP (e.g., reduces the level of polyP) by at least 10% (e.g., at least 20%, at least 30%, at least 40%, at least 50%, at least 60%, at least 70%, at least 80%, at least 90%, or more). In some embodiments, a polyP-neutralizing protein used in the methods described herein inhibits, prevents, or reduces polyP-mediated neurotoxicity by at least 10% (e.g., at least 20%, at least 30%, at least 40%, at least 50%, at least 60%, at least 70%, at least 80%, at least 90%, or more).

In some examples, the polyP-neutralizing protein can be a polyphosphatase such as an exopolyphosphatase (PPX) or polyphosphate binding domain thereof. PPX is a highly processive exopolyphosphatase that degrades polyP by sequentially cleaving phosphate units from the end of a polyP chain. In general, PPX comprises two domains, an N-terminal domain containing the PPX catalytic site and a C-terminal domain containing the polyP binding site. In some examples, the polyP-neutralizing protein can comprise the N-terminal domain of PPX, the C-terminal domain of PPX, or both the N-terminal and C-terminal domains of PPX.

In some examples, the PPX protein or polyphosphate binding domain thereof is from yeast (e.g., S. cerevisiae), bacteria (e.g., E. coli), or mammals (e.g., humans). The amino acid sequence of S. cerevisiae PPX1 (scPPX1) can be found as GenBank Accession No. NP_012071.1, which is provided herein as SEQ ID NO: 1. The amino acid sequence of E. coli PPX (ecPPX) can be found as GenBank Accession No. STL11582.1, which is provided herein as SEQ ID NO: 2.

Amino acid sequence of scPPX1 (SEQ ID NO: 1):
(SEQ ID NO: 1)
MSPLRKTVPEFLAHLKSLPISKIASNDVLTICVGNESADMDSIASAITYSYCQYIYNEGT
YSEEKKKGSFIVPIIDIPREDLSLRRDVMYVLEKLKIKEEELFFIEDLKSLKQNVSQGTE
LNSYLVDNNDTPKNLKNYIDNVVGIIDHHFDLQKHLDAEPRIVKVSGSCSSLVFNYWYEK
LQGDREVVMNIAPLLMGAILIDTSNMRRKVEESDKLAIERCQAVLSGAVNEVSAQGLEDS
SEFYKEIKSRKNDIKGFSVSDILKKDYKQFNFQGKGHKGLEIGLSSIVKRMSWLFNEHGG
EADFVNQCRRFQAERGLDVLVLLTSWRKAGDSHRELVILGDSNVVRELIERVSDKLQLQL
FGGNLDGGVAMFKQLNVEATRKQVVPYLEEAYSNLEE
Amino acid sequence of ecPPX (SEQ ID NO: 2)
(SEQ ID NO: 2)
MPIHDKSPRPQEFAAVDLGSNSFHMVIARVVDGAMQIIGRLKQRVHLADGLGPDNMLSEE
AMTRGLNCLSLFAERLQGFSPASVCIVGTHTLRQALNATDFLKRAEKVIPYPIEIISGNE
EARLIFMGVEHTQPEKGRKLVIDIGGGSTELVIGENFEPILVESRRMGCVSFAQLYFPGG
VINKENFQRARMAAAQKLETLTWQFRIQGWNVAMGASGTIKAAHEVLMEMGEKDGIITPE
RLEKLVKEVLRHRNFASLSLPGLSEERKTVFVPGLAILCGVFDALAIRELRLSDGALREG
VLYEMEGRFRHQDVRSRTASSLANQYHIDSEQARRVLDTTMQMYEQWREQQPKLAHPQLE
ALLRWAAMLHEVGLNINHSGLHRHSAYILQNSDLPGFNQEQQLMMATLVRYHRKAIKLDD
LPRFTLFKKKQFLPLIQLLRLGVLLNNQRQATTTPPTLTLITDDSHWTLRFPHDWFSQNA
LVLLDLEKEQEYWEGVAGWRLKIEEESTPEIAA

In some examples, the polyP-neutralizing protein can be a polyphosphatase such as an endopolyphosphatase PPN1 or polyphosphate binding domain thereof. In some examples, the PPN1 protein or polyphosphate binding domain thereof is from yeast (e.g., s. cerevisiae), bacteria (e.g., E. coli), or mammals (e.g., humans). PPN1 preferentially cleaves long polyP chains in the middle, but the end products of its reaction are triphosphate as well as monomeric Pi, suggesting that PPN1 might also display exophosphatase activity. The amino acid sequence of s. cerevisiae PPN1 (scPPNT) can be found as GenBank Accession No. NP_010740.3, which is provided herein as SEQ ID NO: 3. The amino acid sequence of human Nudt3 can be found as GenBank Accession No. NP_006694.1, which is provided herein as SEQ ID NO: 4.

Amino acid sequence of scPPN1 (SEQ ID NO: 3):
(SEQ ID NO: 3)
MVVVGKSEVRNVSMSRPKKKSLIAILSTCVLFFLVFIIGAKFQYVSVFSKFLDDRGDNES
LQLLNDIEFTRLGLTPREPVIIKDVKTGKERKLHGRFLHITDIHPDPYYVEGSSIDAVCH
TGKPSKKKDVAPKFGKAMSGCDSPVILMEETLRWIKENLRDKIDFVIWTGDNIRHDNDRK
HPRTEAQIFDMNNIVADKMTELFSAGNEEDPRDFDVSVIPSLGNNDVFPHNMFALGPTLQ
TREYYRIWKNFVPQQQQRTFDRSASFLTEVIPGKLAVLSINTLYLFKANPLVDNCNSKKE
PGYQLLLWFGYVLEELRSRGMKVWLSGHVPPIAKNFDQSCYDKFTLWTHEYRDIIIGGLY
GHMNIDHFIPTDGKKARKSLLKAMEQSTRVQQGEDSNEEDEETELNRILDHAMAAKEVFL
MGAKPSNKEAYMNTVRDTYYRKVWNKLERVDEKNVENEKKKKEKKDKKKKKPITRKELIE
RYSIVNIGGSVIPTFNPSFRIWEYNITDIVNDSNFAVSEYKPWDEFFESLNKIMEDSLLE
DEMDSSNIEVGINREKMGEKKNKKKKKNDKTMPIEMPDKYELGPAYVPQLFTPTRFVQFY
ADLEKINQELHNSFVESKDIFRYEIEYTSDEKPYSMDSLTVGSYLDLAGRLYENKPAWEK
YVEWSFASSGYKDD
Amino acid sequence of Nudt3 (SEQ ID NO: 4):
(SEQ ID NO: 4)
MMKLKSNQTRTYDGDGYKKRAACLCFRSESEEEVLLVSSSRHPDRWIVPGGGMEPEEEPS
VAAVREVCEEAGVKGTLGRLVGIFENQERKHRTYVYVLIVTEVLEDWEDSVNIGRKREWF
KIEDAIKVLQYHKPVQASYFETLRQGYSANNGTPVVATTYSVSAQSSMSGIR

Polyphosphatases for use in methods described herein can have exopolyphosphatase activity, endopolyphosphatase activity, or both. Polyphosphatases can be from yeast (e.g., s. cerevisiae such as PPX1 and PPN1 provided in SEQ ID NO: 1 and SEQ ID NO: 3, respectively) or from bacteria (e.g., E. coli such as PPX1 provided in SEQ ID NO: 2) or from mammals (e.g., humans such as Nudt3 provided in SEQ ID NO: 4), although polyphosphatases from other organisms can also be used in methods and compositions described herein.

Although polyphosphatase activity in the mammalian brain was observed decades ago (Kumble, K. D., and Komberg, A. (1996) J. Biol. Chem. 271, 27146-27151), the enzyme(s) responsible for such activity remained unknown until recently when Nudt3 was identified as a mammalian polyphosphatase (Samper-Martin et al. (2021). Cell Reports 37, 110004). These studies demonstrated that Nudt3 has Zn²⁺-dependent polyPase activity and that Nudt3 plays a critical role in vivo in limiting DNA damage and maintaining cell survival upon oxidative stress (Samper-Martin et al. (2021). Cell Reports 37, 110004).

Nudt3 is a member of the MutT/NUDIX (cleaves nucleoside diphosphate linked to some other moiety X) family of proteins, which is an evolutionarily conserved family of 22 hydrolases, including divalent cation-regulated enzymes that hydrolyze diverse dinucleotides and inositol pyrophosphates (PP-InsPs), among a wide range of substrates. DIPPs (diphosphoinositol-pyrophosphate-poly-phosphatases) are a subfamily of the MutT/NUDIX family of proteins that includes Nudt3, Nudt4, Nudt10, and Nudt11. Any MutT/NUDIX family member or other mammalian protein having polyphosphatase activity can be used in methods described herein.

In some embodiments, a polyP-neutralizing protein comprises a full-length polyphosphatase protein such as PPX, PPX1 or PPN1. For example, the polyP-neutralizing protein comprises scPPX1 having an amino acid sequence of SEQ ID NO: 1. In some examples, the polyP-neutralizing protein comprises scPPX1 having an amino acid sequence that is at least 70%, at least 80%, at least 85%, at least 90%, at least 91%, at least 92%, at least 93%, at least 94%, at least 95%, at least 96%, at least 97%, at least 98%, or at least 99% identical to SEQ ID NO: 1.

In another example, the polyP-neutralizing protein comprises ecPPX having an amino acid sequence of SEQ ID NO: 2. In some examples, the polyP-neutralizing protein comprises ecPPX having an amino acid sequence that is at least 70%, at least 80%, at least 85%, at least 90%, at least 91%, at least 92%, at least 93%, at least 94%, at least 95%, at least 96%, at least 97%, at least 98%, or at least 99% identical to SEQ ID NO: 2.

In another example, the polyP-neutralizing protein comprises scPPN1 having an amino acid sequence of SEQ ID NO: 3. In some examples, the polyP-neutralizing protein comprises scPPN1 having an amino acid sequence that is at least 70%, at least 80%, at least 85%, at least 90%, at least 91%, at least 92%, at least 93%, at least 94%, at least 95%, at least 96%, at least 97%, at least 98%, or at least 99% identical to SEQ ID NO: 3.

In another example, the polyP-neutralizing protein comprises Nudt3 having an amino acid sequence of SEQ ID NO: 4. In some examples, the polyP-neutralizing protein comprises Nudt3 having an amino acid sequence that is at least 70%, at least 80%, at least 85%, at least 90%, at least 91%, at least 92%, at least 93%, at least 94%, at least 95%, at least 96%, at least 97%, at least 98%, or at least 99% identical to SEQ ID NO: 4.

In some embodiments, a polyP-neutralizing protein comprises a fragment of a full-length polyP-neutralizing protein. For example, the polyP-neutralizing protein comprises a polyphosphate binding domain of a polyphosphatase such as PPX, PPX1 or PPN1. The fragment of the full-length polyP-neutralizing protein can be any length suitable for binding and neutralizing polyP. For example, the fragment is at least 50 amino acids, at least 100 amino acids, at least 150 amino acids, at least 200 amino acids, at least 250 amino acids, at least 300 amino acids, at least 400 amino acids, at least 500 amino acids, at least 600 amino acids, at least 700 amino acids, at least 800 amino acids, at least 900 amino acids, at least 1000 amino acids, or more amino acids in length.

In some embodiments, the polyP-neutralizing protein comprises a fragment of scPPX1 having an amino acid sequence of SEQ ID NO: 1. In some embodiments, the polyP-neutralizing protein comprises a fragment of scPPX1 that is at least 70%, at least 80%, at least 85%, at least 90%, at least 91%, at least 92%, at least 93%, at least 94%, at least 95%, at least 96%, at least 97%, at least 98%, or at least 99% identical to the full-length amino acid sequence provided in SEQ ID NO: 1.

In some embodiments, the polyP-neutralizing protein comprises a fragment of ecPPX having an amino acid sequence of SEQ ID NO: 2. In some embodiments, the polyP-neutralizing protein comprises a fragment of ecPPX that is at least 70%, at least 80%, at least 85%, at least 90%, at least 91%, at least 92%, at least 93%, at least 94%, at least 95%, at least 96%, at least 97%, at least 98%, or at least 99% identical to the full-length amino acid sequence provided in SEQ ID NO: 2.

In some embodiments, the polyP-neutralizing protein comprises a fragment of scPPN1 having an amino acid sequence of SEQ ID NO: 3. In some embodiments, the polyP-neutralizing protein comprises a fragment of scPPN1 that is at least 70%, at least 80%, at least 85%, at least 90%, at least 91%, at least 92%, at least 93%, at least 94%, at least 95%, at least 96%, at least 97%, at least 98%, or at least 99% identical to the full-length amino acid sequence provided in SEQ ID NO: 3.

In some embodiments, the polyP-neutralizing protein comprises a fragment of Nudt3 having an amino acid sequence of SEQ ID NO: 4. In some embodiments, the polyP-neutralizing protein comprises a fragment of Nudt3 that is at least 70%, at least 80%, at least 85%, at least 90%, at least 91%, at least 92%, at least 93%, at least 94%, at least 95%, at least 96%, at least 97%, at least 98%, or at least 99% identical to the full-length amino acid sequence provided in SEQ ID NO: 4.

In some embodiments, the fragment of the polyP-neutralizing protein comprises the polyphosphate binding domain of a polyphosphatase. For example, the polyP-neutralizing protein comprises the C-terminal polyphosphate binding domain (PPBD) of ecPPX. In some examples, the PPBD of ecPPX comprises amino acids 305 to 513 of SEQ ID NO: 2. See, also Saito et al., 2005 Applied and Environmental Microbiology 71, 5692-5701, the relevant disclosures of which are herein incorporated by reference for the purposes and subject matter referenced herein.

To determine the percent identity of two amino acid sequences, the sequences are aligned for optimal comparison purposes (e.g., gaps can be introduced in one or both of a first and a second amino acid for optimal alignment and non-homologous sequences can be disregarded for comparison purposes). When a position in the first sequence is occupied by the same amino acid residue as the corresponding position in the second sequence, then the molecules are identical at that position (as used herein amino acid “identity” is equivalent to amino acid “homology”). The percent identity between the two sequences is a function of the number of identical positions shared by the sequences, taking into account the number of gaps, and the length of each gap, which need to be introduced for optimal alignment of the two sequences.

Comparison of sequences and determination of percent identity between two sequences can be accomplished using a mathematical algorithm. For example, the percent identity between two amino acid sequences can be determined using the Needleman and Wunsch ((1970) J. Mol. Biol. 48:444-453) algorithm which has been incorporated into the GAP program in the GCG software package (available on the world wide web at gcg.com), using the default parameters, e.g., a Blossum 62 scoring matrix with a gap penalty of 12, a gap extend penalty of 4, and a frameshift gap penalty of 5.

In some embodiments, a polyP-neutralizing protein comprises a mutation, which can be a mutation in a full-length protein or a fragment thereof. The term “mutation” refers to a substitution of a residue within a sequence (e.g., a nucleic acid or an amino acid sequence) with another residue, or a deletion or insertion of one or more residues within a sequence (e.g., a nucleic acid or an amino acid sequence). Mutations are typically described herein by identifying the original residue followed by the position of the residue within the sequence and by the identity of the newly substituted residue. Various methods for making the amino acid mutations provided herein are well known in the art, and are provided by, e.g., Green and Sambrook, Molecular Cloning: A Laboratory Manual (4th ed., Cold Spring Harbor Laboratory Press, Cold Spring Harbor, N.Y. (2012). The polyP-neutralizing protein can comprise any number and/or any type of mutation.

Any of the polyP-neutralizing proteins described herein can be fusion proteins. In such instances, the polyP-neutralizing protein can be fused to a peptide or a protein such as an affinity tag for purification or identification (e.g., a His-tag), a fluorescent tag for visualization (e.g., a GFP-tag), or a cell-penetrating peptide (CPP) for uptake into cells. In some embodiments, the polyP-neutralizing protein is fused to membrane-permeant peptide for secretion and re-uptake into cells (e.g., PTEN-Long).

In some embodiments, a polyP-neutralizing protein comprises an antibody that binds to polyP. As used herein, the term “antibody” refers to a protein that includes at least one immunoglobulin variable domain or immunoglobulin variable domain sequence. For example, an antibody can include a heavy (H) chain variable region (VH) and a light (L) chain variable region (VL). In another example, an antibody includes two heavy chain variable regions and two light chain variable regions. The term “antibody” encompasses full-length antibodies and antigen-binding fragments of antibodies (e.g., single chain antibodies, Fab and sFab fragments, F(ab′)2, Fd fragments, Fv fragments, scFv, and domain antibodies (dAb) fragments. An antibody can have the structural features of IgA, IgG, IgE, IgD, IgM (as well as subtypes thereof).

Any antibody that binds polyP can be used as a polyP-neutralizing protein in methods and compositions described herein. Examples of antibodies that bind polyP include those disclosed in WO2016029001A2, the relevant disclosures of which are herein incorporated by reference for the purposes and subject matter referenced herein.

### (b) Nucleic Acids, Cells, and Vectors

Also within the scope of the present disclosure are nucleic acids encoding polyP-neutralizing proteins, vectors comprising the nucleic acids, and cells comprising the nucleic acids and/or the vectors.

Nucleic acids encoding polyP-neutralizing proteins can be DNA or RNA, single-stranded and/or double-stranded, and can be introduced into a cell in linear or circular form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

Nucleic acids encoding polyP-neutralizing proteins can include natural nucleosides (e.g., adenosine, thymidine, guanosine, cytidine, uridine, deoxyadenosine, deoxythymidine, deoxyguanosine, and deoxycytidine) and/or modified nucleosides such as nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine), chemically modified bases, biologically modified bases (e.g., methylated bases), intercalated bases, modified sugars (e.g., 2-fluororibose, ribose, 2′-deoxyribose, arabinose, and hexose), and/or modified phosphate groups (e.g., phosphorothioates and 5′-N-phosphoramidite linkages).

Nucleic acids encoding polyP-neutralizing proteins can be introduced into cells using any means known in the art, including, without limitation, transformation, transfection (e.g., chemical transfection using calcium phosphate, cationic polymers, or liposomes) or non-chemical (e.g., electroporation, optical transfection, hydrodynamic transfection), and transduction (e.g., viral transduction). In some examples, nucleic acids encoding polyP-neutralizing proteins can be introduced into a subject or cells of the subject using microcapsules, liposomes, or nanoparticles.

Nucleic acids encoding polyP-neutralizing proteins can be introduced into a cell as naked nucleic acid, as nucleic acid complexed with an agent such as a liposome or a nanoparticle, or it can be delivered by viruses (e.g., adenovirus).

Nucleic acids can also include one or more sequences that promote expression of a polyP-neutralizing protein, e.g., one or more promoter sequences; enhancer sequences, e.g., 5′ untranslated region (UTR) or a 3′ UTR; a polyadenylation site; and/or insulator sequences.

Nucleic acids encoding polyP-neutralizing proteins can be operably linked to a promoter, which can be an endogenous promoter or an exogenous promoter. In some examples, the promoter is a constitutive promoter, an inducible promoter, or a tissue-specific promoter. In some examples, the promoter is a neuronal synapsin promoter, a R-actin promoter, or a cytomegalovirus (CMV) promoter. In some embodiments, the promoter is a brain tissue specific promoter, e.g., a neuron-specific or glia-specific promoter. In certain embodiments, the promoter is a promoter of a gene from: neuronal nuclei (NeuN), glial fibrillary acidic protein (GFAP), MeCP2, adenomatous polyposis coli (APC), ionized calcium-binding adapter molecule 1 (Iba-1), synapsin I (SYN), calcium/calmodulin-dependent protein kinase II, tubulin alpha I, neuron-specific enolase and platelet-derived growth factor beta chain. In some embodiments, the promoter is a pan-cell type promoter, e.g., cytomegalovirus (CMV), beta glucuronidase, (GUSB), ubiquitin C (UBC), or rous sarcoma virus (RSV) promoter. The woodchuck hepatitis virus posttranscriptional response element (WPRE) can also be used.

Nucleic acids encoding polyP-neutralizing proteins can be introduced into a cell as part of a vector molecule having additional sequences such as, for example, replication origins, genes encoding antibiotic resistance, and/or transcriptional or translational regulatory sequences such as promoters, enhancers, insulators, internal ribosome entry sites, and/or polyadenylation signals.

In some examples, nucleic acids encoding polyP-neutralizing proteins can be delivered to a cell using an adeno-associated virus (AAV) vector. AAVs are small viruses that integrate site-specifically into the host genome and can therefore deliver a transgene, such as a nucleic acid encoding a polyP-neutralizing protein. Inverted terminal repeats (ITRs) are present flanking the AAV genome and/or the transgene of interest and serve as origins of replication. Also present in the AAV genome are rep and cap proteins which, when transcribed, form capsids which encapsulate the AAV genome for delivery into target cells.

Surface receptors on these capsids which confer AAV serotype, which determines which target organs the capsids will primarily bind, and thus what cells the AAV will most efficiently infect.

AAV has been shown to exhibit long-term episomal transgene expression, and AAV has demonstrated excellent transgene expression in the brain, particularly in neurons. Vectors containing as little as 300 base pairs of AAV can be packaged and can integrate. Space for exogenous DNA is limited to about 4.7 kb. An AAV vector such as that described in Tratschin et al., Mol. Cell. Biol. 5:3251-3260 (1985) can be used to introduce DNA into cells.

A variety of nucleic acids have been introduced into different cell types using AAV vectors (see, for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993). There are numerous alternative AAV variants (over 100 have been cloned), and AAV variants have been identified based on desirable characteristics. In some embodiments, the AAV is AAV1, AAV2, AAV4, AAV5, AAV6, AV6.2, AAV7, AAV8, AAV9, rh.10, rh.39, rh.43 or CSp3; for CNS use, in some embodiments the AAV is AAV1, AAV2, AAV4, AAV5, AAV6, AAV8, AAV9 or AAV-PHP/AAV-PHPeB.

In some embodiments, the AAV also has one or more additional mutations that increase delivery to the target tissue, e.g., the CNS, or that reduce off-tissue targeting, e.g., mutations that decrease liver delivery when CNS, heart, or muscle delivery is intended (e.g., as described in Pulicherla et al. (2011) Mol Ther 19:1070-1078); or the addition of other targeting peptides, e.g., as described in Chen et al. (2008) Nat Med 15:1215-1218 or Xu et al., (2005) Virology 341:203-214 or U.S. Pat. Nos. 9,102,949; 9,585,971; and US20170166926. See also Gray and Samulski (2011) “Vector design and considerations for CNS applications,” in Gene Vector Design and Application to Treat Nervous System Disorders ed. Glorioso J., editor. (Washington, DC: Society for Neuroscience), available at sfn.org/˜/media/SfN/Documents/Short %20Courses/2011%20Short %20Course %20I/2011_S C1_Gray.ashx.

Adeno-associated viruses are among the most frequently used viruses for gene therapy for several reasons. First, AAVs, in some instances, do not provoke an immune response upon administration to mammals, such as humans. Second, AAVs are effectively delivered to target cells, particularly when consideration is given to selecting the appropriate AAV serotype. Finally, AAVs have the ability to infect both dividing and non-dividing cells because the genome can persist in the host cell without integration. These features make AAVs ideal for gene therapy.

Other viral vectors can be used to deliver nucleic acids encoding polyP-neutralizing proteins to a cell. Such viral vectors include, but are not limited to, lentivirus vectors, alphavirus vectors, enterovirus vectors, pestivirus vectors, baculovirus vectors, herpesvirus vectors, Epstein Barr virus vectors, papovavirus vectors, poxvirus vectors, vaccinia virus vectors, and herpes simplex virus vectors.

Also within the scope of the present disclosure are cells comprising nucleic acids encoding polyP-neutralizing proteins. Suitable cells include mammalian cells (e.g., human cells) and bacterial cells (e.g., recombinant bacteria cells for protein expression). In some examples, the cells are glial cells such as astrocytes.

(c) polyP-Neutralizing Polymers

Any polymer (e.g., a linear polymer or a branched polymer) suitable for neutralizing polyP (e.g., neutralizing polyP-mediated neurotoxicity) can be used in methods for treating a neurological disorder as disclosed herein. In some embodiments, a polyP-neutralizing polymer used in the methods described herein neutralizes a polyP biological activity (e.g., polyP-mediated neurotoxicity) by at least 10% (e.g., at least 20%, at least 30%, at least 40%, at least 50%, at least 60%, at least 70%, at least 80%, at least 90%, or more).

Polymers for use in methods described herein can be naturally occurring or synthetic. Polymers can be linear or branched. Non-limiting examples of polyP-neutralizing polymers include dendrimers, polyamines, and combinations thereof.

As used herein, the term “polyamines” refers to a compound that includes at least two amine groups. Non-limiting examples of polyamines include spermidine, norspermidine, spermine, norspermine, putrescine, cadaverine, polyethylenimine (PEI), polybrene, poly-D-lysine, poly-L-lysine, poly-L-arginine, surfen, protamine, diethylenetriamine (DETA), and triethylenetetramine (TETA).

As used herein, the term “dendrimer” refers to a synthetic polymer comprising branched repeating units that emerge from a focal point and possess a large number of exposed anionic, neutral or cationic terminal functionalities on the surface. Non-limiting examples of dendrimers include polyamidoamine (PAMAM) dendrimers, polypropylamine (POPAM) dendrimers, polypropyleneimine (PPI) dendrimers, polyethylenimine (PEI) dendrimers, polyarylether (PAE) dendrimers, polylysine dendrimers, polyester dendrimers, iptycene dendrimers, aliphatic poly(ether) dendrimers, aromatic polyether dendrimers, and universal heparin reversal agents (UHRAs).

Dendrimers can be synthesized in a variety of well-defined molecular weights. Their size and surface functionality (e.g., carboxylic, amine and hydroxyl terminations) is defined by the number of controlled repetitive additions of monomeric units, giving rise to different half (e.g., generation 0.5 dendrimers, generation 1.5 dendrimers, generation 2.5 dendrimers, generation 3.5 dendrimers, generation 4.5 dendrimers, generation 6.5 dendrimers, generation 7.5 dendrimers, generation 8.5 dendrimers, generation 9.5 dendrimers, or more) or full generations (e.g., generation 2 dendrimers, generation 3 dendrimers, generation 4 dendrimers, generation 5 dendrimers, generation 6 dendrimers, generation 7 dendrimers, generation 8 dendrimers, generation 9 dendrimers, generation 10 dendrimers, or more).

Any generation of dendrimer can be used in methods and compositions described herein. For example, when the polyP-neutralizing polymer comprises a PAMAM dendrimer, the PAMAM dendrimer can be a generation 1 (GI) PAMAM dendrimer, a generation 2 (G2) PAMAM dendrimer, a generation 3 (G3) PAMAM dendrimer, a generation 4 (G4) PAMAM dendrimer, a generation 5 (G5) PAMAM dendrimer, a generation 6 (G6) PAMAM dendrimer, a generation 7 (G7) PAMAM dendrimer, a generation 8 (G8) PAMAM dendrimer, a generation 9 (G9) PAMAM dendrimer, a generation 10 (G10) PAMAM dendrimer, or a combination thereof.

In another example, when the polyP-neutralizing polymer comprises a PAMAM dendrimer, the PAMAM dendrimer can be a generation 0.5 (G0.5) PAMAM dendrimer, a generation 1.5 (G1.5) PAMAM dendrimer, a generation 2.5 (G2.5) PAMAM dendrimer, a generation 3.5 (G3.5) PAMAM dendrimer, a generation 4.5 (G4.5) PAMAM dendrimer, a generation 5.5 (G5.5) PAMAM dendrimer, a generation 6.5 (G6.5) PAMAM dendrimer, a generation 7.5 (G7.5) PAMAM dendrimer, a generation 8.5 (G8.5) PAMAM dendrimer, a generation 9.5 (G9.5) PAMAM dendrimer, or a combination thereof.

UHRAs are formed by assembling multifunctional cationic groups into the core of a dendritic polymer, which are then shielded from non-specific interactions using a protective layer of short-chain polyethylene glycol (PEG).

Non-limiting examples of UHRAs that can be used as provided herein are provided in International Application No. WO2015179958A1, the relevant disclosures of which are herein incorporated by reference for the purposes and subject matter referenced herein. In some examples, the UHRA is selected from the group consisting of UHRA-1, UHRA-2, UHRA-5, UHRA-6, UHRA-7, UHRA-8, UHRA-9, UHRA-10, UHRA-11, UHRA-13, UHRA-14, and UHRA-15.

Also within the scope of the present disclosure are dendrimer complexes comprising dendrimers that can be similar or different than other dendrimers in the complex. For example, the dendrimer complex can comprise PAMAM dendrimers or PAMAM dendrimers and POPAM dendrimers. In some embodiments, the dendrimer or dendrimer complex can further include an additional agent such as a therapeutic agent.

### (d) Pharmaceutical Compositions

A polyP-neutralizing molecule (e.g., a polyP-neutralizing protein, nucleic acids encoding a polyP-neutralizing protein, vectors comprising such nucleic acids, host cells comprising the encoding nucleic acids and/or the vectors, and polymers) can be mixed with a pharmaceutically acceptable excipient (carrier) to form a pharmaceutical composition for use in treating a neurological disorder (e.g., ALS, FTD, ALS-FTD). “Acceptable” means that the excipient must be compatible with the active ingredient of the composition (and preferably, capable of stabilizing the active ingredient) and not deleterious to the subject to be treated. Pharmaceutically acceptable excipients (carriers), including buffers, are well known in the art. See, e.g., Remington: The Science and Practice of Pharmacy 20^{th} Ed. (2000) Lippincott Williams and Wilkins, Ed. K. E. Hoover.

The pharmaceutical compositions to be used in the present methods can comprise pharmaceutically acceptable carriers, excipients, or stabilizers in the form of lyophilized formulations or aqueous solutions. Acceptable carriers, excipients, or stabilizers are nontoxic to recipients at the dosages and concentrations used, and may comprise buffers (e.g. phosphate, citrate, and other organic acids); antioxidants (e.g., ascorbic acid, methionine); preservatives (e.g., octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins (e.g., serum albumin, gelatin, immunoglobulins); hydrophilic polymers (e.g. polyvinylpyrrolidone); amino acids (e.g., glycine, glutamine, asparagine, histidine, arginine, lysine); monosaccharides, disaccharides, and other carbohydrates including glucose, mannose, or dextrans; chelating agents (e.g., ETDA) sugars (e.g., sucrose, mannitol, sorbitol); salt-forming counter-ions (e.g., sodium); metal complexes (e.g., Zn-protein complexes); and/or non-ionic surfactants (e.g., TWEEN™, PLURONICS™, polyethylene glycol (PEG)).

In some examples, the pharmaceutical composition described herein comprises liposomes containing a polyP-neutralizing molecule (e.g., a polyP-neutralizing protein or nucleic acid encoding such protein or a polymer). Such liposomes can be prepared using any method known in the art.

Alternatively, or in addition to, a polyP-neutralizing molecule can be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (e.g., liposomes, albumin microspheres, microemulsions, nanoparticles and nanocapusules) or in macroemulsions.

In other examples, the pharmaceutical compositions described herein can be formulated in sustained-release format. Suitable examples of sustained-release preparations include semi-permeable matrices of solid hydrophobic polymers containing a polyP-neutralizing molecule, which matrices are in the form of shaped articles, e.g., films or microcapsules. Examples of sustained-release matrices include polyesters, hydrogels (e.g., poly(2-hydroxyethyl-methacrylate), poly(vinyl alcohol)), polylactides, copolymers of L-glutamic acid and ethyl-L-glutamate, non-degradable ethylene-vinyl acetate, degradable lactic acid-glycolic acid copolymers, sucrose acetate isobutyrate, and poly-D-(−)-3-hydroxybutyric acid.

The pharmaceutical compositions to be used for in vivo administration must be sterile. This is readily accomplished by, for example, filtration through sterile filtration membranes. Pharmaceutical compositions described herein can be placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient (i.e., a polyP-neutralizing molecule) can be mixed with a pharmaceutical carrier, e.g., conventional tableting ingredients such as corn starch, lactose, sucrose, sorbital, talc, stearic acid, magnesium stearate, dicalcium phosphate or gums, and other pharmaceutical diluents, e.g. water, to form a solid preformulation composition containing a homogeneous mixture of an active ingredient. When referring to these preformulation compositions as homogeneous, it is meant that the active ingredient is dispersed evenly throughout the composition so that the composition can be readily subdivided into equally effective unit dosage forms such as tablets, pills, and capsules. This solid preformulation composition is then subdivided into unit dosage forms of the type described above containing from 0.1 to about 500 mg of the active ingredient of the present invention. The tablets or pills can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coating, such materials including a number of polymeric acids and mixtures of polymeric acids with such materials as shellac, cetyl alcohol, and cellulose acetate.

Suitable surface-active agents include, but are not limited to, non-ionic agents, such as polyoxyethylenesorbitans (e.g., Tween™ 20, 40, 60, 80 or 85) and other sorbitans (e.g., Span™ 20, 40, 60, 80, or 85). Compositions with a surface-active agent can comprise between 0.05% and 5% surface-active agent (e.g., between 0.1% and 2.5%). It will be appreciated that other ingredients may be added, for example mannitol or other pharmaceutically acceptable vehicles, if necessary.

Suitable emulsions can be prepared using commercially available fat emulsions, such as Intralipid™, Liposyn™, Infonutrol™, Lipofundin™, and Lipiphysan™. The active ingredient (i.e., a polyP-neutralizing molecule) can be either dissolved in a pre-mixed emulsion composition or alternatively it may be dissolved in an oil (e.g., soybean oil, safflower oil, cottonseed oil, sesame oil, corn oil, or almond oil) and an emulsion formed upon mixing with a phospholipid (e.g., egg phospholipids, soybean phospholipids or soybean lecithin) and water. It will be appreciated that other ingredients can be added, for example glycerol or glucose, to adjust the tonicity of the emulsion. Suitable emulsions will typically contain up to 20% oil, for example, between 5% and 20%. The fat emulsion can comprise fat droplets between 0.1 and 1.0 μm, e.g., 0.1 and 0.5 μm, and have a pH in the range of 5.5 to 8.0.

In addition to a polyP-neutralizing molecule, the pharmaceutical compositions described herein may comprise one or more additional therapeutic agents such as those described herein.

Relative amounts of the active ingredient (e.g., a polyP-neutralizing molecule), the pharmaceutically acceptable excipient, and/or any additional ingredients or any additional therapeutic agents in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

I. Applications of polyP in Neurological Disorders

The present disclosure provides, in some aspects, uses of polyP in the diagnosis, prognosis, and treatment of neurological disorders such as a stroke, a brain and/or CNS injury, neuroinflammation, a neurodegenerative disease (e.g., ALS, FTD, ALS/FTD, PD, and HD), or a combination thereof. Diagnostic, prognosis, and treatment methods described herein, in some examples, involve detection of polyP in a biological sample using any means known in the art or described herein.

Any sample that may contain polyP can be analyzed by the assay methods described herein. In some examples, the sample may be from an in vitro assay, e.g., from an in vitro cell culture (e.g., an in vitro cell culture of human astrocytes derived from iPSC as described here). In other examples, the sample to be analyzed by the assay methods described herein is a sample obtained from a subject.

As used herein, a “sample” refers to a composition that comprises biological materials including, but not limited to, plasma, tissue, cells, and/or fluid from a subject. A sample includes both an initial unprocessed sample taken from a subject as well as subsequently processed, e.g., partially purified or preserved forms. In some embodiments, the sample is a CSF sample. In some embodiments, multiple (e.g., at least 2, 3, 4, 5, or more) samples may be collected from a subject, over time or at particular time intervals, for example, to assess the disease progression or evaluate the efficacy of a treatment. A sample can be obtained from a subject using any means known in the art.

The term “subject” or “patient” can be used interchangeably and refers to a subject who needs the analysis as described herein. In some embodiments, the subject is a human or a non-human mammal (e.g., cat, dog, horse, cow, goat, or sheep). In some embodiments, a subject has or is at risk for a neurological disorder, e.g., a stroke, a brain injury, neuroinflammation, a neurodegenerative disease, or a combination thereof. Such a subject can exhibit one or more symptoms associated with a neurological disorder. Alternatively or in addition to, such a subject can have one or more risk factors for a neurological disorder, e.g., a genetic mutation associated with a neurological disorder.

Detection of polyP in a sample includes assessing the presence or absence, quantity, amount or level of polyP, size of polyP fragments, and length of polyP fragments within a sample, including the derivation of qualitative or quantitative levels of polyP, or otherwise evaluating the values and/or categorization of polyP in a sample from a subject.

Any assay known in the art or described herein can be used to detect polyP in a sample from a subject or in the body of the subject. Non-limiting examples of methods for detecting polyP that can be used as provided herein are provided in Christ et al. Methods for the Analysis of Polyphosphate in the Life Sciences, Anal. Chem. 2020, 92, 6, 4167-4176, the relevant disclosures of which are herein incorporated by reference for the purposes and subject matter referenced herein.

For example, polyP can be detecting using dyes (e.g., DAPI, JC-D7, or JC-D8), enzymes (e.g., polyP kinase (PPK)), spectroscopy (e.g., NMR, IR, absorption and fluorescence spectroscopy), mass spectrometry, electrophoresis (e.g., PAGE), chromatography, microscopy, or combinations thereof.

### (a) Diagnostic Methods

Aspects of the present disclosure provide methods for diagnosis of a neurological disorder (e.g., a stroke, a brain injury, neuroinflammation, a neurodegenerative disease, or a combination thereof) based on detection of polyP in a sample (e.g., a CSF sample) collected from a subject (e.g., a human patient having a neurological disorder).

In some embodiments, the amount by which the level (or score) in the subject is more than the reference level (or score) is sufficient to distinguish a subject from a control subject, and optionally is a statistically significantly more than the level (or score) in a control subject. In cases where the level (or score) of polyP in a subject being equal to the reference level (or score) of polyP, the “being equal” refers to being approximately equal (e.g., not statistically different).

Suitable reference values can be determined using methods known in the art, e.g., using standard clinical trial methodology and statistical analysis. The reference values can have any relevant form. In some cases, the reference comprises a predetermined value for a meaningful score or level of polyP, e.g., a control reference level that represents a normal level of polyP, e.g., a level in an unaffected subject or a subject who is not at risk of developing a neurological disorder, and/or a disease reference that represents a level of polyP associated with risk of developing a neurological disorder.

The predetermined level or score can be a single cut-off (threshold) value, such as a median or mean, or a level or score that defines the boundaries of an upper or lower quartile, tertile, or other segment of a clinical trial population that is determined to be statistically different from the other segments. It can be a range of cut-off (or threshold) values, such as a confidence interval. It can be established based upon comparative groups, such as where association with risk of developing disease or presence of disease in one defined group is a fold higher, or lower, (e.g., approximately 2-fold, 4-fold, 8-fold, 16-fold or more) than the risk or presence of disease in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk. In some embodiments, the predetermined level or score is a level or score determined in the same subject, e.g., at a different time point, e.g., an earlier time point.

The control level as described herein can be determined by various methods. In some embodiments, the control level can be obtained by performing a known method. In some embodiments, the control level can be obtained by performing the same assay used to determine the level of polyP in a sample from a subject. In some embodiments, the control level can be obtained by performing a method described herein. In some embodiments, the control level can be obtained from members of a control population and the results can be analyzed by, e.g., a computational program, to obtain the control level (a predetermined level) that represents the level of polyP in the control population.

By comparing the level of polyP in a sample obtained from a subject to the reference value as described herein, it can be determined as to whether the subject has or is at risk for a neurological disorder (e.g., a stroke, a brain injury, or a neurodegenerative disease). For example, if the level of polyP of the subject is elevated from the reference value (e.g., increased as compared to the reference value), the candidate subject might be identified as having or at risk for a neurological disorder. In other examples, if the level of polyP of the subject is reduced from the reference value (e.g., decreased as compared to the reference value), the candidate subject might be identified as having or being at risk for a neurological disorder.

As used herein, “an elevated level or a level above a reference value” means that the level of polyP is higher than a reference value, such as a predetermined threshold or a level of polyP in a control sample.

An elevated level of polyP includes a polyP level that is, for example, 1%, 5%, 10%, 20%, 30%, 40%, 50%, 60%, 70%, 80%, 90%, 100%, 150%, 200%, 300%, 400%, 500% or more above a reference value. An elevated level of polyP also includes increasing a phenomenon from a zero state (e.g., no or undetectable polyP in a sample) to a non-zero state (e.g., some or detectable polyP in a sample).

As used herein, “a decreased level or a level below a reference value” means that the level of polyP is lower than a reference value, such as a predetermined threshold or a level of polyP in a control sample.

An decreased level of polyP includes a polyP level that is, for example, 1%, 5%, 10%, 20%, 30%, 40%, 50%, 60%, 70%, 80%, 90%, 100%, 150%, 200%, 300%, 400%, 500% or more below a reference value. A decreased level of polyP also includes decreasing a phenomenon from a non-zero state (e.g., some or detectable polyP in a sample) to a zero state (e.g., no or undetectable polyP in a sample).

A subject having or a risk for having a neurological disorder, as identified using methods described herein, can be treated with any appropriate therapy including those known in the art and those disclosed herein. For example, when the subject has or is a risk for having ALS, the subject can be treated with riluzole or rasagiline and a polyP-neutralizing molecule.

### (b) Prognostic Methods

Levels of polyP in a sample from a subject can be used for various prognostic purposes, for example, monitoring disease progress, assessing efficacy of a treatment, and/or identifying patients suitable for treatment. For example, when assessing efficacy of a treatment, multiple samples can be collected from a subject to whom a treatment is performed either before and after the treatment or during the course of the treatment. If the level of polyP decreases after the treatment or over the course of the treatment (the level of polyP in a later collected sample as compared to that in an earlier collected sample), remains the same or decreases, it indicates that the treatment is effective.

If the subject is identified as not responsive to the treatment, a higher dose and/or frequency of dosage of the therapy are administered to the subject identified. In some embodiments, the dosage or frequency of dosage of the therapy is maintained, lowered, or ceased in a subject identified as responsive to the treatment or not in need of further treatment. Alternatively, a different treatment can be applied to the subject who is found as not responsive to the first treatment.

### (c) Treatment Methods

To practice the treatment methods described herein, an effective amount of any appropriate therapy can be administered to a subject (e.g., a human patient) in need of the treatment via any suitable route. Appropriate therapies for a neurological disorder include those known in the art and those disclosed herein (e.g., polyP-neutralizing molecules such as PPX1, UHRA10, and G4-PAMAM-NH2).

Treatment of a neurological disorder includes treatment using a polyP-neutralizing molecule alone or in combination with an additional therapy for a neurological disorder. Therapies for a neurological disorder include, but are not limited to, polyP-neutralizing molecules (e.g., polyP-neutralizing proteins or encoding nucleic acids such proteins or polymers such as polyamines and dendrimers), anticoagulants (e.g., warfarin, apixaban, dabigatran, edoxaban, and rivaroxaban), antihypertensive agents (e.g., beta-blockers, angiotensin-converting enzyme (ACE) inhibitors, calcium channel blockers, and alpha blockers), sodium channel blockers (e.g., riluzole), edaravone, antidepressants (e.g., selective serotonin reuptake inhibitors (SSRIs)), antipsychotics (e.g., olanzapine, and quetiapine), dopamine (e.g., levodopa and carbidopa), dopamine agonists (e.g., pramipexole, ropinirole, rotigotine, and apomorphine), MAO B inhibitors (e.g., selegiline, rasagiline, and safinamide), catechol O-methyltransferase (COMT) inhibitors (e.g., entacapone and opicapone), anticholinergics (e.g., benztropine and trihexyphenidyl), gene therapies (e.g., any gene editing technology such as CRISPR-based gene editing systems for targeting a disease associated genetic defect or mutation), and surgical therapies (e.g., deep brain stimulation).

When the therapy includes genetic and epigenetic editing, the (epi)genetic editing can be performed using any suitable technology, for example, clustered regularly interspaced short palindromic repeats (CRISPR), transcription activator-like effector nucleases (TALENs), zinc-finger nucleases (ZFNs), and/or homing endonucleases or meganucleases technologies.

In some examples, the therapy can include (epi)genetic editing of a gene, for example, a gene involved in synthesis or degradation of polyP. For example, when a gene is involved in polyP synthesis (e.g., polyP kinase (PPK)), the therapy can include disrupting that gene to reduce its expression. In another example, when a gene is involved in polyP degradation (e.g., PPX, Nudt3), the therapy can include activating that gene to enhance its expression.

Alternatively, or in addition to, the therapy can include a nucleic acid that reduces expression of a gene involved in synthesis of polyP. Non-limiting examples of such nucleic acids include short interfering nucleic acid (siNA), short interfering RNA (siRNA), double-stranded RNA (dsRNA), micro-RNA (miRNA), and short hairpin RNA (shRNA) molecules.

The subject to be treated by the methods described can be a human patient having or at risk for having a neurological disorder. Examples of a neurological disorder include, but are not limited to, stroke, brain injury (e.g., a traumatic brain injury), neuroinflammation, neurodegenerative diseases (e.g., ALS, FTD, ALS-FTD, PD, and HD), and combinations thereof.

The subject to be treated by the methods described herein can be a mammal such as a human. Mammals include, but are not limited to, farm animals, sport animals, pets, primates, horses, dogs, cats, mice, and rats. A human subject who needs the treatment can be a human patient having or a risk for having a neurological disorder such as stroke, brain injury (e.g., a traumatic brain injury), neuroinflammation, neurodegenerative diseases (e.g., ALS, FTD, ALS-FTD, PD, and HD), or combinations thereof.

A subject having or at risk for having a neurological disorder can be identified by routine medical examination, e.g., laboratory tests (e.g., blood tests), magnetic resonance imaging (MRI), computerized tomography (CT) scans, electromyogram (EMG), nerve conduction studies, biopsy (e.g., muscle biopsy), spinal tap, and cerebral angiogram.

A subject having or at risk for having a neurological disorder might show one or more symptoms of a neurological disorder, e.g., headache, difficulty walking and/or speaking, slurred speech, difficulty swallowing, muscle cramps and/or twitching, muscle atrophy, tremor, vision problems, paralysis or numbness, weakness, cognitive or behavioral changes, impaired posture and balance, loss of consciousness, nausea, convulsions or seizures, and dilation of one or both pupils of the eyes.

A subject at risk for having a neurological disorder can be a subject having one or more of the risk factors for that neurological disorder. For example, the risk factors associated with ALS and FTD include (a) family history of ALS or FTD, (b) age, (c) sex, (d) genetic abnormalities, (e) smoking, (f) exposure to toxins, (g) diabetes, (h) hypertension, and (i) obesity.

An effective amount of a therapy can be administered to a subject (e.g., a human) in need of the treatment via any suitable route, such as intravenous administration (e.g., as a bolus or by continuous infusion over a period of time), intramuscular, intraperitoneal, intracerebrospinal, subcutaneous, intra-articular, intrasynovial, intrathecal, oral, inhalation, or topical routes.

“An effective amount” as used herein refers to the amount of each active agent required to confer therapeutic effect on the subject, either alone or in combination with one or more other active agents. Effective amounts vary, as recognized by those skilled in the art, depending on the particular condition being treated, the severity of the condition, the individual patient parameters including age, physical condition, size, gender and weight, the duration of treatment, the nature of concurrent therapy, if any, the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

Empirical considerations such as the half-life of an agent will generally contribute to the determination of the dosage. Frequency of administration can be determined and adjusted over the course of therapy, and is generally, but not necessarily, based on treatment and/or suppression and/or amelioration and/or delay of a neurological disorder such as stroke, brain injury (e.g., a traumatic brain injury), and neurodegenerative diseases such as ALS, FTD, ALS-FTD, PD, and HD. Alternatively, sustained continuous release formulations of therapeutic agent may be appropriate. Various formulations and devices for achieving sustained release are known in the art.

As used herein, the term “treating” refers to the application or administration of a composition including one or more active agents to a subject who has a neurological disorder, a symptom of a neurological disorder, and/or a predisposition toward a neurological disorder, with the purpose to cure, heal, alleviate, relieve, alter, remedy, ameliorate, improve, or affect the disorder, the symptom of the neurological disorder, and/or the predisposition toward the neurological disorder.

Alleviating a neurological disorder includes delaying the development or progression of the disease, and/or reducing disease severity. Alleviating the disease does not necessarily require curative results.

As used herein, “delaying” the development of a disease (e.g., a neurological disorder) means to defer, hinder, slow, retard, stabilize, and/or postpone progression of the disease. This delay can be of varying lengths of time, depending on the history of the disease and/or individuals being treated. A method that “delays” or alleviates the development of a disease and/or delays the onset of the disease is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques known in the art. However, development also refers to progression that may be undetectable. For purposes of this disclosure, development or progression refers to the biological course of the symptoms. “Development” includes occurrence, recurrence, and onset. As used herein, “onset” or “occurrence of a neurological disorder includes initial onset and/or recurrence.

In some embodiments, the therapy is administered one or more times to the subject. In some embodiments, the therapy comprises two or more types of therapies that can be administered as part of a combination therapy for treatment of a neurological disorder (e.g., a combination therapy comprising polyP-neutralizing molecule (e.g., polyP-neutralizing proteins or encoding nucleic acids such proteins or polymers) and riluzole or rasagiline.

The term combination therapy, as used herein, embraces administration of these agents in a sequential manner, that is wherein each therapeutic agent is administered at a different time, as well as administration of these therapeutic agents, or at least two of the agents, in a substantially simultaneous manner.

Sequential or substantially simultaneous administration of each agent can be affected by any appropriate route including, but not limited to, oral routes, intravenous routes, intramuscular routes, subcutaneous routes, and direct absorption through mucous membrane tissues. The agents can be administered by the same route or by different routes. For example, a first agent can be administered orally, and a second agent can be administered intravenously.

As used herein, the term “sequential” means, unless otherwise specified, characterized by a regular sequence or order, e.g., if a dosage regimen includes the administration of a first therapeutic agent and a second therapeutic agent, a sequential dosage regimen could include administration of the first therapeutic agent, before, simultaneously, substantially simultaneously, or after administration of the second therapeutic agent, but both agents will be administered in a regular sequence or order. The term “separate” means, unless otherwise specified, to keep apart one from the other. The term “simultaneously” means, unless otherwise specified, happening or done at the same time, i.e., the agents of the invention are administered at the same time. The term “substantially simultaneously” means that the agents are administered within minutes of each other (e.g., within 10 minutes of each other) and intends to embrace joint administration as well as consecutive administration, but if the administration is consecutive it is separated in time for only a short period (e.g., the time it would take a medical practitioner to administer two agents separately). As used herein, concurrent administration and substantially simultaneous administration are used interchangeably. Sequential administration refers to temporally separated administration of the agents described herein.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

In order that the invention described may be more fully understood, the following examples are set forth. The examples described in this application are offered to illustrate the methods and compositions provided herein and are not to be construed in any way as limiting their scope.

### Materials and Methods

The following materials and methods were used in the Examples set forth herein.

### Control and Transgenic Mice

Hemizygous transgenic mice carrying mutant human SOD1^{G93A} (high copy number; B6SJL; Cat. #002726), wild-type human SOD1^{WT} (B6SJL; Cat. #002297) and TARDBP^{A315T} (C57BL/6J; Cat. #010700) transgenes were obtained from Jackson Laboratories (Bar Harbor, Maine, USA). Hemizygous transgenic mice (background B6SJLF/J) carrying C9ORF72 hexanucleotide repeats (˜500) in intron were generated by Robert Brown (Peters et al., 2015). All mice were maintained and bred at the animal facility of Universidad Andres Bello. The presence of the human SOD1transgene, the human TARDBP transgene and the human C9ORF72 transgene were validated by polymerase chain reaction as previously described. Transgenic males were crossed with WT females (B6SJL or C57BL/6J) from the corresponding background; these non-transgenic littermates (NTg) were used as internal controls for all experiments. The SOD1^{G93A} (mutSOD1) mice, but not the hSOD1WT (wtSOD1) mice, develop signs of neuromuscular deficits (tremor of the legs and loss of extension reflex of the hind paws) starting at 3 months of age and have an average lifespan of 133-147 days. Mice carrying TDP43^{A315T} (mutTDP43) develop similar loss of motor function between 2-4 months and do not survive to the age of 4 months. Mice carrying C9ORF72 hexanucleotide repeats (˜500) (mutC9ORF72) show pathological hallmarks (RNA foci and dinucleotide peptides) of ALS and FTD as well as mild cognitive deficits at 6 and 9 months of age. Sprague-Dawley rats were originally obtained from the Pontifical Catholic University of Chile (Santiago) and maintained and bred at the animal facility of Universidad Andres Bello.

### Primary Mouse Astrocyte Cultures

Primary astrocyte cultures and ACM were prepared as previously described from mutSOD1 (Fritz et al., 2013 Journal of Neurophysiology 109, 2803-2814; Rojas et al., 2014, Frontiers in Cellular Neuroscience 8, 24), mutTDP43 (Rojas et al., 2014 Frontiers in Cellular Neuroscience 8, 24) and mutC9ORF72 (Jury et al., 2020 Clin Epigenetics 12(1):32) spinal cords derived from neonatal transgenic mice. Briefly, cultures of astrocytes were prepared from whole spinal cords of these transgenic ALS mice at P1-2. Non-transgenic astrocytes from littermates (Ctrl) and astrocytes expressing wtSOD1 (wtSOD1) were used as controls. Cells were maintained in DMEM (Hyclone, Cat. #SH30081.01) containing 10% FBS (Gibco, Cat. #16000-044), 1% L-glutamine (Gibco, Cat. #25030-081) and 1% penicillin-streptomycin (Gibco, Cat. #15140-122) at 37° C., 5% C02. Cultures reached confluence after −3 weeks. Residual microglia were removed by shaking overnight, leaving astrocyte cultures enriched for S100b and Aldh1L1. Astrocytes were used to determine intracellular polyP content (FIGS. 1A-1F) or to generate ACM (FIGS. 2A-2B, FIGS. 5A-5D, and FIGS. 6A-6I).

### Astrocyte-Conditioned Media (ACM)

As previously described (Fritz et al., 2013, Journal of Neurophysiology 109, 2803-2814; Rojas et al., 2014 Frontiers in Cellular Neuroscience 8, 24), after primary spinal cord astrocyte cultures reached confluence, media was replaced by neuronal growth media: 70% MEM (Gibco, Cat. #11090-073), 25% Neurobasal media (Gibco, Cat. #21103-049), 1% N2 supplement (Gibco, Cat. #17502-048), 1% L-glutamine (Gibco, Cat. #25030-081), 1% penicillin-streptomycin (Gibco, Cat. #15140-122), 2% horse serum (Gibco, Cat. #15060-114; lot 1517711) and 1% sodium pyruvate (Gibco, Cat. #11360-070). After 7 days, ACM was collected, supplemented with 4.5 mg ml/L D-glucose (final concentration), filtered and stored at −80° C. These media were used to evaluate i) ACM-polyP deposition assay on neuronal plasma membranes (FIGS. 2A-2B), and ii) survival of motoneurons (FIGS. 5A-5D, and FIGS. 6A-6I). For both assays, ACM was diluted. The ACM-mutSOD1, ACM-mutTDP43, ACM-wtSOD1 and their respective ACM-Ctrls were diluted 9-fold. The ACM-mutC9ORF72 and its ACM-Ctrl were diluted 8-fold. At the selected dilutions, ACM derived from the astrocytes expressing the ALScausing genes robustly killed motoneurons and resulted in detectable deposits of polyP at neuronal membranes relative to the control media.

### Primary Ventral Spinal Cord Cultures

Pregnant Sprague-Dawley rats were deeply anesthetized with C02 and primary spinal cultures were prepared from E14 pups as described (Fritz et al., 2013, Journal of Neurophysiology 109, 2803-2814; Rojas et al., 2014 Frontiers in Cellular Neuroscience 8, 24). Briefly, cultures of ventral spinal cords were prepared and maintained in neuronal growth media: 70% MEM (Gibco, Cat. #11090-073), 25% Neurobasal media (Gibco, Cat. #21103-049), 1% N2 supplement (Gibco, Cat. #17502-048), 1% L-glutamine (Gibco, Cat. #25030-081), 1% penicillin-streptomycin (Gibco, Cat. #15140-122), 2% horse serum (Gibco, Cat. #15060-114; lot 1517711) and 1% sodium pyruvate (Gibco, Cat. #11360-070). Cultures were maintained for 7-9 DIV at 37° C. under 5% C02 and supplemented with 45 μg/ml E18 chick leg extract; media was renewed every 3 days.

### Motoneuron Survival Assay

To measure survival of motoneurons and interneurons in ventral spinal cord cultures treated with ACM, cultures were immunolabeled and counted according to standard procedures known in the art. Briefly, primary spinal cultures were fixed at 7 DIV with 4% paraformaldehyde, and immunostained with a rabbit polyclonal antibody against MAP2 (1:200; Invitrogen; Cat. #OSM00030W) to label all neurons (intemeurons plus motoneurons) and with a mouse monoclonal SMI-32 antibody (1:600, Abcam; Cat. #ab187374) to reveal the presence of unphosphorylated neurofilament-H, which is expressed specifically in motoneurons in spinal cord cultures, previously it was found that WT primary spinal cultures typically contain at least 8-10% motoneurons until 12 DIV (Sepulveda et al., 2010 Journal of Neurophysiology 103, 1758-1770). Fluorescently labeled neurons were visualized with epifluorescent illumination on an Olympus IX81 microscope (equipped with a Q-Imaging Micropublisher 3.3 Real-Time Viewing camera) using a 20× objective; MAP2- and SM132-positive neurons were counted off-line using Fuji ImageJ. Per condition, at least 12 randomly chosen fields were analyzed to calculate the percentage of SMI-32-positive motoneurons within the total number of MAP2-positive cells. Each condition was replicated in 3-4 independent cultures.

Intracellular polyP Staining Assays with DAPI-polyP, JC-D8 and recPPBD in Primary Astrocyte Cultures

To evaluate intracellular polyP levels in primary astrocyte cultures, polyP labeling assays were performed with DAPI-polyP, JC-D8, or recPPBD. All steps were performed at room temperature, unless indicated. All images were taken on a Leica TCS SP8 confocal microscope with a 63× oil objective (NA=1.4; HC PL APO CS2) and a z-step of 0.5 μm optical sections (velocity scan 600 Hz; resolution 1024×1024 pixels, equivalent to 185 μm×185 μm):
(i) DAPI-polyP: cultured astrocytes were fixed with 4% paraformaldehyde for 20 min, permeabilized with 0.1% Triton-X-100 for 20 min (only if additional immunofluorescent labeling was used), loaded with the fluorescent dye DAPI (0.1 μg/ml, Sigma, Cat. #D9542) in PBS for 30 min, carefully washed 3 times and mounted on coverslips with non-fluorescing fluoromount-G™ (Electron Microscopy Sciences, Cat. #17984-25). Note that it is not recommended to use mounting media that already includes DAPI (e.g., DAPI-Fluoromount-G or Prolong Gold antifade mountant with DAPI). The following laser excitation (Ex) and emission (Em) wavelengths were used to detect DAPI-polyP (Ex: 488 nm and Em: 510-560 nm) and DAPI-DNA (Ex: 405 nm and Em: 410-470 nm).
(ii) JC-D8: live cultured astrocytes were incubated with HEPES-buffered salt solution (HBSS) containing the fluorescent probe JC-D8 (5 μM) for 30 min in the C02 incubator at 37° C. Next, cells were carefully washed 3 times with HBSS and fixed with 4% paraformaldehyde for 20 min. Cells were permeabilized with 0.1% Triton-X-100 for 20 min (only if additional immunofluorescent labeling was used), loaded with the nuclear dye To-Pro-3 (termed here TOPRO3) (1:2000, Cat. #T3605, Invitrogen), washed and mounted on coverslips with nonfluorescing fluoromount-G™. The following laser excitation and emission wavelengths were used to detect JC-D8 (Ex: 488 nm and Em: 510-560 nm) and TOPRO3 (Ex: 638 nm and Em: 643-776 nm).

(iii) recPPBD: cultured astrocytes were fixed with 4% paraformaldehyde for 20 min, permeabilized with 0.1% Triton-X-100 for 20 min, washed with PBS, blocked with goat serum and treated with the yeast recombinant PPBD protein (recPPBD containing an Xpress epitope tag) (20-30 μg/ml) in goat serum for 30 min. RecPPBD was produced in E. coli BL21 bacteria, exactly as described (Saito et al., 2005 Applied and Environmental Microbiology 71, 5692-5701). Next, cultures were incubated with a mouse anti-Xpress antibody (1:500, Cat. #R910-25, Invitrogen) overnight at 4° C., washed, and incubated with an Alexa-conjugated goat anti-mouse antibody (1:500, Alexa⁴⁸⁸, Invitrogen, Cat. #A-11029) for 1.5 h. Cells were finally loaded with the nuclear dye TOPRO3, washed 3 times and mounted on coverslips with non-fluorescing fluoromount-G™. The following laser wavelengths were used to detect recPPBD-Xpress-Alexa⁴⁸⁸ (Ex: 488 nm and Em: 550 nm) and TOPRO3 (Ex: 638 nm and Em: 643-776 nm).

For quantifying the intracellular polyP signal in individual astrocytes, the polyP fluorescent signal intensity detected by recPBD, JC-D8 or DAPI-polyP was measured from the maximum intensity projection of confocal z-stack images (4 μm total) by Fiji ImageJ software (8 bits, measuring intensity from 0 to 225). Specifically, the signal located within a circular region of interest (ROI) was analyzed; this ROI had a diameter twice the size of the analyzed nucleus. Four randomly chosen fields (185 μm×185 μm) were analyzed per independent experiment for each experimental condition; per field, 2 isolated astrocytes (containing a nuclear diameter ranging from 10-14 μm) were randomly quantified per condition. Each condition was replicated in 3-4 independent cultures, leading to the analysis of 24-32 astrocytes per condition.

Colocalization of DAPI-polyP, JC-D8 and recPPBD in mutSOD Astrocytes

To determine the colocalization between (i) recPPBD and DAPI-PolyP, and (ii) recPPBD and JC-D8 in mutSOD1 astrocytes, labeling was performed as described in the above section, with some modifications. For both co-labeling experiments, the Xpress epitope of recPPBD was detected with Alexa⁵⁴⁶-conjugated goat anti-mouse antibody (1:500, Alexa⁵⁴⁶, Invitrogen, Cat. #A-11003). The following laser wavelengths were used for the co-labeling of (i) recPPBD and DAPIpolyP: recPPBD-Xpress-Alexa⁵⁴⁶ (Ex: 552 nm and Em: 560-620 nm), DAPI-PolyP (Ex: 488 nm and Em: 510-560 nm), and DAPI-DNA (Ex: 405 nm and Em: 410-470 nm), and (ii) recPPBD and JC-D8: recPPBD-Xpress-Alexa546 (Ex: 552 nm and Em: 560-620 nm), JC-D8 (Ex: 488 nm and Em: 510-560 nm), and TOPRO (Ex: 638 nm and Em: 643-776 nm). All images were taken on a Leica TCS SP8 confocal microscope with a 63× oil objective and 3× digital zoom (NA=1.4; HC PL APO CS2) and a z-step of 0.1 μm optical sections (velocity scan 600 Hz; resolution 1024×1024 pixels, equivalent to 62 μm×62 μm). Pearson correlation coefficients were calculated by Huygens Professional software. Each colocalization was analyzed in 6-8 astrocytes per condition.

### Acm-polyP Deposition Assay on Neuronal Plasma Membranes

Ventral spinal cord cultures (2 DIV) were infected with AAV2-synapsin-tdTomato. At 7-8 DIV, live cultures were incubated with HEPES-buffered salt solution (HBSS) containing the fluorescent probe JC-D8 (5 μM) for 30 min in the C02 incubator at 37° C. Next, cultures were treated with ACM, feeding media or polyPL for 5 min, carefully washed 3 times with HBSS, fixed with 4% paraformaldehyde for 20 min and mounted on coverslips with non-fluorescing fluoromount-G™. The following laser excitation and emission wavelengths were used to detect JC-D8 (Ex: 488 nm and Em: 510-560 nm) and tdTomato (Ex: 638 nm and Em: 653-776 nm). All images were taken on a Leica TCS SP8 confocal microscope with a 63× oil objective (NA=1.4; HC PL APO CS2) and a z-step of 0.5 μm optical sections (velocity scan 600 Hz; resolution 1024×1024 pixels, equivalent to 185 μm×185 μm). For quantifying the polyP deposition on individual tdTomato-positive spinal cord neurons, the polyP fluorescent signal intensity detected by JC-D8 was measured from the maximum intensity projection of confocal z-stack images (4-5 μm total) by Fiji ImageJ software (8 bits, measuring intensity from 0 to 225). To measure the polyP signal on the soma of tdTomato-positive spinal cord neurons, ROIs were drawn on the cell bodies. Six randomly chosen fields (185 μm×185 μm) were analyzed per independent experiment for each experimental condition; per field, 6 isolated tdTomato-positive neurons were randomly quantified per condition. Each condition was replicated in 3 independent cultures.

### PolyP Neutralizing Molecules

PolyP was degraded with recPPX/PPase from the Phosfinity kit (Aminoverse) or with CIP (NEB, Cat #M0525). To sequester and neutralize polyP in astrocyte-conditioned media, the following molecules were used: (i) recPPBD (20-30 μg/ml), produced in E. coli BL21 bacteria as described (Saito et al., 2005); (ii) UHRA9 and UHRA10 (5-500 nM), donated (Smith et al., 2012; Travers et al., 2014); (iii) G4-PAMAM-NH₂ (0.1-1 μg/ml, Cat. #412449, Sigma). The capacity of these molecules to bind polyP was tested in TBE urea-PAGE gels (not shown).

Polyp Fluorescent Staining Assays in Fixed Mouse Spinal Cord Sections with recPPBD

MutSOD1 (4 mo), mutTDP43 (2 mo) and mutC9ORF72 (9 mo) mice and their corresponding non-transgenic littermates were anesthetized with ketamine/xylazine and then transcardially perfused with Ringer's solution followed by a perfusion with cold 4% paraformaldehyde. The spinal cord was carefully removed and post-fixed in 4% paraformaldehyde overnight at room temperature. Next, samples were cryoprotected in a sucrose gradient (10, 20 and 30%) for 3 days at 4° C. Sagittal spinal cord sections (40 μm thick) were cut using a cryostat (Leica, CM 152S, Germany). Lumbar spinal cord sections were washed with PBS and incubated next with blocking/permeabilization solution (3% donkey serum and 3% BSA in 0.5% Triton X-100 in PBS) for 4 hr at room temperature and then incubated with recPPBD (30 μg/ml) containing an Xpress epitope tag for 40 min at room temperature. Next, slices were washed three times with PBS and incubated with the following primary antibodies: anti-NeuN (1:200, rabbit, Cat. #MAB377, Millipore), anti-GFAP (1:1000, rabbit, Cat. #G3893, Sigma) and anti-Xpress (1:500, mouse, Cat. #R910-25, Invitrogen) in blocking/permeabilization solution overnight at 4° C. After rinsing 3 times with 0.5% Triton X-100 in PBS, the spinal cord slices were incubated with Alexaconjugated goat secondary antibodies and the nuclear dye TOPRO3 (1:1000, Cat. #T3605, Invitrogen) in blocking/permeabilization solution for 2 hr at room temperature. The following Alexa-conjugated goat secondary antibodies were used: for recPPBD (1:500, anti-mouse Alexa488, Cat. #A-11029, Invitrogen), for GFAP and NeuN (1:500, anti-rabbit Alexa546 Cat. #A-11029 and anti-rabbit Alexa546, Cat. #A-11081, Invitrogen). Finally, slices were washed twice with 0.5% Triton X-100 in PBS and once with PBS and mounted on coverslips with nonfluorescing fluoromount-G™ (Electron Microscopy Sciences, Cat. #17984-25). Images of the ventral horn of the lumbar spinal cord were taken on a Leica TCS SP8 confocal microscope with a 63× oil objective (NA=1.4; HC PL APO CS2) and a z-step of 0.5 μm optical sections (velocity scan 600 Hz; resolution 1024×1024 pixels, equivalent to 185 μm×185 μm). The following laser wavelengths were used to detect RecPPBD-Xpress-Alexa⁴⁸⁸ (Ex: 488 nm and Em: 510-550 nm), NeuN-Alexa⁵⁴⁶ (Ex: 552 nm and Em: 564-620 nm), GFAP-Alexa⁵⁴⁶ (Ex: 552 nm and Em: 564-620 nm).

Maximum intensity projections of confocal z-stack images of whole cells (containing 12-17 images) were analyzed. Fiji ImageJ software (8 bits, measuring intensity from 0 to 225) was used to quantify the intracellular recPPBD-polyP fluorescence intensity. Specifically, the recPPBD-polyP signal located within the soma of individual NeuN-positive and GFAP-positive cells was quantified by drawing an ROI on the cell bodies of these cells. Three randomly chosen fields (200 μm×200 μm) within ventral horn were analyzed per independent experiment for each experimental condition; per field, 8-10 NeuN-positive and 8-10 GFAP-positive cells were randomly quantified. Each condition was replicated in 3-4 mice.

PolyP Immunohistochemical Staining Assays with recPPBD on Human Post-Mortem Spinal Cord Sections

The autopsy spinal cord samples from 9 ALS cases and 5 non-neurologic control subjects were provided by the Target ALS Human Postmortem Tissue Core. Formalin-fixed paraffin-embedded tissue sections (3 μm) were deparaffinized in Xylene and rehydrated in a graded alcohol series (100%, 96%, 70% and 50%) for 10 min in each bath and rinsed in running tap water before pre-treatment with sodium citrate buffer 0.01M, pH 6 for 10 min at 90° C. for antigen retrieval. Samples were washed 3 times 5 min each in a solution of 0.01M PBS 0.15% Triton X-100 and incubated with 0.3% H₂O₂ at room temperature for 30 min, then incubated 1 h with blocking solution containing 5% BSA in 0.01M PBS 0.15% Triton X-100 and 1 h with recPPBD (30 μg/ml) followed by overnight incubation at 4° C. with the primary antibody anti-Xpress (1:500, Cat. #R910-25, Invitrogen) diluted in blocking solution. After being rinsed 3 times with 0.01M PBS 0.15% Triton X-100, sections were incubated for 1 h with a mouse biotinylated secondary antibody (1:250, Cat. #BA-9200, Vectastain, ABC Elite kit PK-6101, Vector Laboratories) and diluted in blocking solution followed by 1 h incubation with avidin-biotin-peroxidase complex (1:125, Vectastain, ABC Elite kit PK-6101, Vector Laboratories). Finally, a 30% H₂O₂ solution was added in the presence of 3,3′-diaminobenzidine (DAB, 1 mg/ml) to visualize the antigen-antibody complex. Staining for cresyl violet was performed in tissue sections to identify the different regions in human spinal cord. Next, images were captured on a BX51 microscope (Olympus; Tokyo, Japan) with a halogen light source and light-balancing daylight. Photoshop software version 13.0 (Adobe Systems Inc., San Jose, CA) and ImageJ Fiji version 1.52i (National Institutes of Health, USA) were used to analyze and post-process images.

To count the number of motoneuron-like cells with their respective areas, two pictures per ventral spinal cord section were taken for every sample at 10× objective (682 μm×512 μm area) and all cells with ≥110 μm² area and a visible nucleolus typically found in motoneurons were counted. To count the number of glial-like cells with their respective area, two pictures per ventral spinal cord section were taken for every sample at 100×(350 μm×260 μm area). For the glia-like cells, all cells with an area ranging from 5 μm² to 16 μm² were counted and the following phenotypes as previously described (Forsberg et al., 2011 Acta Neuropathologica 121, 623-634): astrocytes, characterized by their round nuclear shape; microglia, characterized by their round nuclear shape but smaller area than astrocytes; and oligodendrocytes, characterized by their elongated nuclear shape. To measure the size of the soma of cells, the cresyl violet staining served to draw an ROI using Fiji ImageJ software. The drawn ROIs were used to quantify the DAB staining of the soma of individual cells. To accurately quantify only the intensity of the DAB signal without the interference of the cresyl violet staining the images were processed as described (Linden et al., 2015 J Histochem Cytochem. 63, 233-243). Briefly, brown color (corresponding to DAB signal) was selected from the background area (blue, white and non-tissue areas) with the use of the eyedropper tool and transformed in grayscale 8 bits images with intensity ranging from 0 to 225. Ten pictures at 100× were analyzed per sample. At least 10 motoneuron-like cell and 25 glia-like cells were analyzed per subject in the lumbar spinal cord samples. In thoracic and cervical spinal cord samples, 2-6 motoneuron-like cell and 25-30 glia-like cells were analyzed per subject.

Human iPSC-Induced Astrocytes Cultures and ACM

We analyzed fully reprogrammed iPSC lines previously generated by retroviral transduction with the four Yamanaka factors (OCT4, SOX2, KLF4, and cMYC) from skin biopsies derived from an ALS/FTD subject (75 years old, termed patient) carrying a A90V mutation in TARDBP (TDP43A90V) and a healthy subject (56 years old), a control family member without mutations (termed control) (Zhang et al., 2013). Maintenance of iPSCs and differentiation to NPCs were performed as previously described (Almeida et al., 2012; Zhang et al., 2013). Note that because same reagents are used for different differentiation steps, the company and catalogue number is indicated only the first time. Briefly, iPSCs were maintained in feeder-free conditions using mTeSR1 medium and EBs were generated and maintained in suspension for one week in EB differentiation media: knock-out (KO) DMEM/F12 (Gibco Cat. #12660-012) media supplemented with 10% KO serum replacement (Gibco Cat. #10828-028), 1× GlutaMax (Gibco Cat. #35050-061), 1×NEAA (Gibco Cat. #11140-050), and 2-mercaptoethanol (Sigma-Aldrich Cat. #M3148). To obtain rosette-shaped neuroepithelial cells, the EBs were plated on poly-L-ornithine-laminin-coated plates (Sigma-Aldrich Cat. #P4957, Cat. #L2020) and were grown for one week in neural induction media: KO-DMEM/F12 supplemented with N2 (Gibco Cat. #17502-048), NEAA, 2 μg/mL heparin (Sigma-Aldrich Cat. #H3149), and 10 ng/mL DFGF (Gibco Cat. #PHG0021). Fully formed rosettes were manually isolated under the microscope, re-plated on matrigel-coated plates (Corning Cat. #354277) and grown for one more week in neural expansion media: neurobasal supplemented with Glutamax, NEAA, B-27 (Gibco Cat. #17504-044), and PFGF. The disaggregation of rosettes using accutase (EMD Millipore Cat. #SCR005) generated a stable, proliferative, monolayer culture of NPCs. As previously described, NPCs were differentiated to astrocytes by culturing cells first for 2 weeks in astrocyte precursor medium (Shaltouki et al., 2013): KO DMEM/F12, 1× StemPro NSCs Supplement (Gibco Cat. #A10508-01), 10 ng/mL Activin A (Gibco Cat. #PHC9564), 10 ng/mL Heregulin 10 (R&D Systems Cat. #377-HB-050), 200 ng/mL IGF1 (R&D System Cat. #P 291-Gi-200), 20 ng/mL PFGF, 20 ng/mL EGF (Gibco Cat. #PHG0311), 1× GlutaMAX. And then for 2 additional weeks in astrocyte maturation and maintenance medium (Malik et al., 2014): DMEM/F12, B27, 10 ng/mL Heregulin, 5 ng/mL BMP2 (BioVision Cat. #4577-50) and 2 ng/mL CNTF (R&D Systems Cat. #257-NT-010). Next, astrocyte cultures were fixed with 4% paraformaldehyde and immunostained with a rabbit polyclonal antibody against EAAT2/GLT1 (1:50; Invitrogen, Cat #PA5-17099), a mouse monoclonal antibody against Cx43 (1:200; Invitrogen, Cat #13-8300), a rabbit polyclonal antibody against S1000 (1:1000; Dako, Cat #Z0311) and a mouse monoclonal antibody against ALDH1L1 (1:50; NeuroMab, Cat #75-140).

After primary human iPSC-induced astrocyte cultures reached day 28 of differentiation, media was replaced by neuronal growth media as described for mouse astrocytes: 70% MEM (Gibco, Cat. #11090-073), 25% Neurobasal media (Gibco, Cat. #21103-049), 1% N2 supplement (Gibco, Cat. #17502-048), 1% L-glutamine (Gibco, Cat. #25030-081), 1% penicillin-streptomycin (Gibco, Cat. #15140-122), 2% horse serum (Gibco, Cat. #15060-114; lot 1517711) and 1% sodium pyruvate (Gibco, Cat. #11360-070). After 7 days with this media (day 35 of differentiation), ACM was collected, supplemented with 4.5 mg ml/L D-glucose (final concentration), filtered and stored at −80° C. The control-ACM and human TDP43-ACM used to evaluate MN survival was diluted 6-fold. At the selected dilution, ACM derived from the patient astrocytes robustly killed MNs.

Quantification of polyP in ACM

To extract polyP from ACM, we developed a modified version of a phenol-chloroform (P/C) extraction protocol originally devised for yeast (Bru et al, 2014; Christ and Blank, 2018). To measure the polyP-Pi content in ACM, isolated polyP was degraded with recPPX/PPase (Christ and Blank, 2018) available from the commercial Phosfinity kit (Aminoverse); we then quantified the liberated free Pi by colorimetrically by Malachite green or quantified the polyP-polymer fluorometrically with the JC-D8 probe. For both methods, standard curves were generated using synthetic polyPL (in water) to calculate the ACM polyP-Pi/polymer concentration and estimate polyP recovery ratio.

Briefly, to isolate and purify polyP from ACM, 400 μl of ACM or synthetic polyPL (as control) were incubated with 1× trypsin (Gibco, Cat #15090046) for 1 h at 37° C. The digested samples were transferred to a 1.5 ml microcentrifuge tube with 300 μl of phenol (Sigma, Cat #77607) and 40 μl of 2% SDS. The samples were mixed by inverting the tubes four times and vortexing for 5 sec. Then, the samples were incubated for 5 min at 65° C. and immediately transferred to ice for 1 min. Next, 300 μl of chloroform:isoamilic alcohol (24:1) was added to each sample and again the solutions were mixed and vortexed. A low-density phase lock gel (Dow Corning®) was added to each tube and the samples were centrifugated at 13.000×g for 2 min at RT. The top aqueous phase was transferred to a new tube and 350 μl of chloroform:isoamilic alcohol (24:1) was added. After mixing, vortexing and add anew low-density phase lock gel, the samples were centrifugated at 13.000×g for 2 min at RT. Finally, the top aqueous phase was desalted using a Zeba™ spin desalting column (Thermo Scientific, Cat #89883). The final eluate was recovered and quantified for polyP-Pi or polyP-polymer detection.

For polyP-Pi quantification, final eluates from ACM (and synthetic polyPL) were digested with recPPX/PPase enzyme mix (Amminoverse, Phosphinity kit) for 1 h at 37° C. The Pi determination was performed with the Malachite green phosphate Assay kit (BioAssay Systems, Cat #POMG-25H). Briefly, 50 μl of each sample was incubated with 12.5 μl of mix A:B reagents (100:1) and incubated for 30 min at RT. Then, absorbance was read at 620 nm in a Synergy LX multi-model reader (BioTek). Finally, in every sample, Pi concentration was estimated subtracting PPX/PPase un-digested from the PPX/PPase digested situation. For polyP-polymer quantification, 100 μl of ACM (and synthetic polyPL) samples were incubated with 100 μl of JC-D8 10 μM in a 20 mM HEPES pH 7.4-1% DMSO buffer, for 15 min at RT. Each sample was transferred to a dark 96-wells microplate (Thermo Scientific, Cat #165305) and exposed for 20 sec to an UV transilluminator. Then, a Synergy H1 hybrid multi-model reader (BioTek) was set at 390 nm for excitation and emission was recorded from 420 to 700 nm to detect the JC-D8 fluorescence. The maximum value between 500 and 560 nm was selected to estimate the polyP-polymer concentration. The recovery ratio for polyPL was 73±9% (n=4).

Quantification of polyP in Human CSF

To extract polyP from human CSF, we adapted an analytical silica column protocol originally devised for tissues (Lee et al., 2018). To measure the polyP content, isolated polyP was degraded with recPPX/PPase (Christ and Blank, 2018), available from the commercial Phosfinity kit (Aminoverse); we then used Malachite green to quantify liberated free Pi. Briefly, 1 ml aliquots of individual human CSF samples were treated with 0.5 mg/ml proteinase K (Millipore, Cat #70663-4) and 10 mM EDTA (pH 8) (Thermo Scientific, Cat #AAJ15694AE) and then were heated for 2 h at 56° C. in a hybridization oven. Next, CSF was then separated into 4×1.5 ml Eppendorf tubes, and 2 volumes of binding buffer was added: 5 M guanidine thiocyanate (Thermo Fisher Scientific cat #AM9422) in 50 mM Tris-Cl pH 6.8 (Fisher Scientific, Cat #BP152-5), and 25 mM EDTA, 0.9 M Na-acetate (pH 5.3) (Thermo Fisher Scientific cat #AM9740), 1% BME). Tubes were then placed on a heat block at 95° C. for 10 min, cooled down to RT, and 2 volumes of 100% ethanol were added. Samples were mixed by inversion, and the content of each tube was loaded twice on EconoSpin column (Epoch Lifescience) and centrifuged (10,000×g, 30 sec, RT). The flow-through was discarded, and 0.7 ml of washing buffer I (1 M guanidine thiocyanate in 80% ethanol) was added. The solution was centrifuged (11,000×g, 1 min, RT) and the flow-throw discarded. The column was washed twice with washing buffer II (150 mM NaCl, 10 mM Tris-Cl pH 7.5 in 80% ethanol), centrifuged (12,000×g, 2 min, RT) and the column was transferred into a clean 1.5 ml microcentrifuge tube. PolyP was eluted by incubating the filter with elution buffer (60 μl of 10 mM Tris-HCl (pH 8)) for 15 min at RT and centrifuged (12,000×g, 2 min, RT). The elution step was repeated twice more, with a 10 min incubation in the two. The final eluates from the 4 tubes containing the extracts from the same CSF samples were pooled, and concentrated by using an Amicon-ultracel 3K filter (cat #UFC5003-96, Millipore). The ultrafiltrate was brought to a final volume of about 190 μl, and analyzed for its polyP content. During this process, each analyzed CSF sample was concentrated about 5-fold; the volumes were measured to determine the concentration factor needed to calculate the final polyP concentration after completing the digestion and the Pi measurements (see below). To quantify Pi, we used the Phosfinity kit (Amminoverse) to digest polyP, according to the manufacturer's instructions, with the following modifications. Briefly, 110 μl of concentrated eluate was incubated at 37° C. for 1 h in the presence and absence of 1.1 μl of a recombinant PPX1/PPase1 and 55 μl of enzyme buffer provided in the kit. The incubation was stopped by adding 10 μM EDTA and the free Pi liberated by the enzymatic reaction was measured in triplicate by loading 50 μl aliquots in 96 multi-wells plates (Greiner Bio-One cat #650101), together with 12.5 μl of the Malachite green reagent (BioAssay System, Cat #POMG-25H). The plate was shaken for 30 sec and incubated for 30 min at RT; the absorbance (ABS) at 620 nm was read using a Spectramax M3 plate reader (Molecular Devices). CSF Pi content was calculated by subtracting the ABS of the recPPX/PPase-untreated samples from the ABS of recPPX/PPase-treated samples. The Pi standard calibration curve generated for each assay was used to calculate the polyP concentration for each averaged ABS/sample. Finally, the amount of polyP was corrected for the concentrating factor of each sample and was expressed in [μM]. As a control protocol (P-ctrl), 2.5 μM PolyPL in artificial CSF (125 mM NaCl, 25 mM NaH3CO4, 2.5 mM KCl, 1.25 mM Na-phosphate buffer (pH 7.2), 2 mM CaCl₂), 1 mM MgCl2, 0.1 mg/ml BSA) was incubated with 0.5 mg/ml proteinase K and 10 mM EDTA (pH 8) and was processed as for all the other samples to assess the recovery ratio as well as the ability of the recPPX/PPase to digest the polyP in the extracts. The recovery ratio for artificial CSF was 75±3% (n=6).

### Intracellular Ca²⁺ Imaging Assays

Ratiometric Ca²⁺ imaging experiments with fura-2AM were performed in primary spinal cord neurons as described (Fritz et al., 2013). Briefly, neurons were incubated with 5 μM fura-2-AM (F1221, Invitrogen-Thermo Fisher Scientific, Waltham, MA, USA) dissolved in standard extracellular solution supplemented with 0.02% pluronic acid (P6867, Invitrogen-Thermo Fisher Scientific, Waltham, MA, USA) for 50 min at 37° C. in darkness. Fluorescence measurements were made using an inverted Nikon Ti microscope fitted with a 12-bit cooled ORCA C8484-03G02 CCD camera (Hamamatsu, Hamamatsu City, Japan). Fura-2 was excited at 340 nm and 380 nm at 0.5 Hz with a Polychrome V monochromator (Till Photonics), with exposure times no longer than 40 ms; the emitted fluorescence was filtered with a 510 nm longpass filter. Calibrated ratios were displayed online with HCImage v2 software (Hamamatsu, Japan). A solution containing elevated K⁺ (50 mM KCl) was perfused at the end of the protocol to determine the viability of the neurons in the entire field. Only cells showing a [Ca²⁺]ᵢ increase in response to a 50 mM elevation in extracellular K⁺ were included in the analysis. The “calcium activity fingerprint graph” was generated graphPad.

Additionally, Ca²⁺ imaging was also performed in spinal cord neurons using the genetically encoded calcium indicator GCaMP6s. For this, 3-4 DIV neurons were infected with AAV1/2 viral particles containing the insert hSyn1-mRuby2-GSG-P2A-GCaMP6s-WPRE-pA (Addgene). At 6-7 DIV, Ca²⁺ frequency was recorded using an epifluorescence microscope (Nikon TE2000e, 20× objective and Andor Zyla 5.5 camera). Images were taken every 50 ms for 1-1.5 min using an excitation wavelength at 480 nm and emission at 510 nm. For the quantification of the calcium event frequency, the soma of isolated spinal cord neurons was selected and analyzed using the “Z profiler” plugin (imagej.nih.gov/ij/plugins/zprofiler.html) of the ImageJ Fiji software. Each intensity peak was counted manually. For both calcium imaging assays, at least 40 neurons per culture were analyzed. Each condition was replicated in 3-4 independent cultures.

### TIRF Imaging

To visualize vesicular fusion events, total internal reflection fluorescence microscopy (TIRF) was used. The imaging setup was equipped with a 100× objective (Olympus, Japan), and an EM-CCD camera (Luca-S, Andor, Ireland). Ctrl and mutSOD1 primary astrocytes were loaded with JC-D8 (5 μM) for 30 min at room temperature in HEPES-buffered salt solution (HBSS). Videos were acquired using WinFluor software and later converted and analyzed using Z Profiler plugin in ImageJ software. For the TIRF assays, 4 neurons per condition were quantified.

### Transcriptomic Assay

Spinal cord cultures (4 DIV) were treated for 90 min with polyPL (100 μM), mutSOD1-ACM or wtSOD1-ACM. Total RNA was extracted from four independent cultures using RNeasy kit (Qiagen). RNA quality was verified on a bioanalyzer before library preparation. TruSeq stranded mRNA protocol (Illumina) was used to generate libraries that were sequenced on an Illumina HiSeq4000 sequencing instrument. Single-end 50 bp reads were aligned to the mouse genome and differentially expressed genes were analyzed using NOISeq method (BGI). Functional analysis of differentially expressed genes dataset was performed using DAVID database of those genes with a cut-off fold change of 2. Data are expressed as polyPL vs. wtSOD1-ACM and mutSOD1-ACM vs. wtSOD1-ACM.

### A AV9 Vector Design and Packaging

AAV9 vectors were produced by triple transient transfection of HEK293T cells and purified by iodixanol gradient centrifugation. AAV vectors carry two AAV2 inverted terminal repeats flanking a transgene expression cassette composed of the cytomegalovirus enhancer fused to a chicken R-actin promoter/rabbit R globin intron, cDNA of the yeast scPPX1 (PPX) gene, fused to GFP, and an SV40 poly A. Two different GFP-PPX versions were generated: (i) PPX-GFP (Abramov et al., 2007) (pTR-CB-Cyto.GFP.PPX; 8.73×10¹² vg/ml), and (ii) PLong-GFP-PPX (pTR-CB-PLong.GFP.PPX; 5.12×10¹² vg/ml), where the native GFP-PPX chimeric protein was genetically fused to the PTEN membrane-permeant peptide (Hopkins et al., 2013 Science 341, 399-402).

### Electrophysiological Assay

Action potentials (APs) were recorded by conventional whole-cell patch clamp. For the recording of spontaneous Aps, neurons were hold under the current-clamp mode (I=0 pA). Borosilicate glass pipettes (World Precision Instruments, Cat. #TW150F-4) were pulled to 2-5 MΩ resistance and filled with internal solution containing (mM): 105 K-Gluconate, 35 KCl, 8.8 NaCl, 0.5 EGTA 10 HEPES, 30 sucrose (pH 7.2 adjusted with KOH). The bath solution (artificial cerebrospinal fluid solution, ACSF) contained (mM): 150 NaCl, 5.4 KCl, 2 CaCl₂), 2 MgCl₂, 10 HEPES, 10 glucose (pH 7.4 adjusted with NaOH). Data were acquired at room temperature (20-23° C.) using an Axopatch 200B (Molecular Device) or EPC7 (HEKA) amplifier and pClamp 10.7 software (Axon Instruments), low pass-filtered at 1 or 5 kHz, and digitized at 10 kHz using a 1322a Digidata (Axon Instruments). Spontaneous APs were recorded in the absence of injected current for at least 2 minutes at each paired condition in cells with R_{access}<10 MΩ. Cells were discarded when they had no APs (measured the first 2 min), showed excessive hyperactivity (firing frequency higher than 1 Hz) or when displaying an unstable baseline. The frequency of AP was calculated off-line using the Threshold Search option of the Event Detection of Clampfit 10.7 software. The baseline was manually adjusted and the AP was counted if the membrane potential changed 30 mV or more. The number of APs 1 min before and 1 min after ACM application were calculated. All neurons with frequencies between 0.1 and 1.0 Hz and with no change in R_{access} were analyzed. For each electrophysiological analysis, at least 10 neurons were analyzed per condition.

### Drosophila Strains

repoGal4 (BL: 7415), mutSOD1 (is UAS-hSOD1^{A4V}, human mutant SOD1, BL: 33607), wtSOD1 (is UAS-hSOD1, human wild-type SOD1, BL: 33606), dSOD1 (Drosophila wild-type SOD1 isoform, BL:33605) were obtained from the Bloomington Stock Centre. The human SOD original lines were deposited by Bonini's lab in BL (Watson et al., 2008). w1118 and UAS-GFP was a gift of John Ewer.

### Drosophila Life Span Assays

For survival assays, flies were grown and aged at 29° C. Flies were reared at a maximum of 20 individuals per tube, transferred every other day into fresh media and dead flies were counted. Flies were maintained on standard commeal/molasses/yeast/agar media with 80% humidity on a 12:12-h light-dark cycle. For each experiment the repoGal4 line alone or driving GFP was used as control. For lifespan assays of transgenic fly lines, we performed all statistical analysis using IBM® SPSS® Statistics. The logrank test was used to compare survival distributions between groups. At least three independent cohorts were used for each genotype with controls and queries running in parallel each time.

### Fly Climbing Assays

Measurements were taken from three independent aged cohorts (50-60 aleatory individuals each time). Fifteen to 20 flies per assay were placed in a clean tube 24 h before the experiment. Tubes were tapped and recorded. The trajectory of flies in the tube was traced as a segmented line. Kymograph was used to calculate the speed of each moving object. The slope of each trajectory in the tube was calculated manually using the end point coordinates, multiplied by the length of the tube (in mm) and divided by the speed of the movie (30 frames/s) to obtain an individual speed in mm/s. All procedures were performed on ImageJ. An average speed per genotype per time point was obtained from at least two different crosses. Average speeds were compared using unpaired t-test.

### Drug Treatment and Fly Rescue Experiments

UHRA10, donated (Smith et al., 2012, Blood 120, 5103-5110; Travers et al., 2014 Blood 124, 3183-3190), was dissolved in water at final 500 nM. 100 μl covering the whole surface of the food of the rearing tube was used to ensure equal access to the drug and adult flies were transferred after absorption of the liquid the same days, some hours later. Control flies were treated in the same manner using water (drug vehicle). Flies were flipped every 2 days. At least three independent cohorts were used for each genotype with controls and queries running in parallel each time.

### Jc-D8 Staining and Imaging in Drosophila

For JCD8 staining, 3- and 30-day old fly brains were hand dissected, fixed in formalin 4% for 5 min, and JC-D8 (10 μM) was added for 1 hour at RT, and TOPRO3 (Invitrogen, T3605; dilution 1:5000) for 10 min. The brains were washed with PBS and then mounted for visualization. Experiments were performed 3 times and individuals for each sample were taken from independent aged cohorts. Images of Drosophila lobal globes were taken on a Leica TCS SP8 confocal microscope with a ×63 oil objective and ×2 digital zoom, and a z-step of 0.5 μm optical sections. The following laser wavelengths were used to detect TOPRO3 (Ex 638 nm and Em 643-776 nm) and JC-D8 (Ex 488 nm and Em 510-560 nm). Maximum intensity projections of confocal z-stack images (containing 8-10 stacks) were analyzed.

### Western Blot of Drosophila Brain Samples

For each sample twenty frozen head flies were homogenized in 50 μl of RIPA buffer. For total hSOD1 detection, samples were boiled 10 min at 95° C. with laemmli buffer and run on 10% SDS-PAGE. For misfolded hSOD1 detection, samples were treated under non-reducing condition (no SDS, no b-mercaptoethanol and no heat), run on 10% naïve PAGE and transferred to nitrocellulose membranes following standard procedures for western blot. Membranes were blocked in blocking buffer (PBS, 0.1% tween, 5% milk) and were incubated overnight with primary antibodies for hSOD1 (Abcam, ab238052; 1:1000), misfolded hSOD1 (Medimabs, MM-0070-p; 1:1000), GFP (Invitrogen, A-11122; 1:2000) and Repo (DSHB, dilution 1:100). The latter was used as loading control. The next day, membranes were washed and incubated with HRP-conjugated second antibody at room temperature for 2 hr. Experiments were performed three times with 20-30 heads per sample obtained from independent aged cohorts.

### Statistical Analysis

Statistical analyses were performed using GraphPad Prism 5 software. Oneway ANOVA followed by the Bonferroni post-hoc was utilized when making multiple (three or more) comparisons. Student's t-test (rodent samples) and Mann-Whitney U-test (human samples) were performed when two populations were examined. In all figures, data is reported as mean±S.E.M.; *P<0.05, **P<0.01, ***P<0.001 compared to control. #P<0.05, ##P<0.01, and ###P<0.001, compared to mutSOD1-ACM or polyPL (as indicated in legends).

### Example 1: Intracellular polyP Level is Elevated in Primary Mouse ALS/FTD Astrocytes that Carry Mutations in SOD1, TARDBP and C9ORF72

To confirm the presence of polyP (FIG. 1A) in ALS/FTD astrocytes, three methods that use fluorescence microscopy were evaluated to detect this polymer. (i) The first method used was staining with DAPI (4′,6-diamidino-2-phenylindole) (FIG. 1B), a non-selective fluorescent dye that labels DNA (DAPI-DNA) at 456 nm emission (blue), but that shifts its emission to 526 nm (green), when interacting with polyP (DAPI-polyP). A caveat here is that polyanions other than intracellular polyP (such as RNA and DNA) may interfere with the fluorometric quantification of DAPI-polyP. (ii) Therefore, the JC-D8 dye (FIG. 1B), which exhibits a sharp increase in fluorescence intensity upon binding to polyP, but not to other polymers (e.g., RNA and DNA) or biological phosphate-containing molecules (i.e., ATP) was also used. (iii) The third method used involved a recombinant polyP binding domain (termed “recPPBD”) (FIG. 1B), derived from the Escherichia coli exopolyphosphatase (PPX) and that is fused to an Xpress epitope tag; this tag permits detection of polyP in yeast and mammalian cells (shown herein) as identified by an immunostaining assay (on fixed and permeabilized cells) with an antibody against Xpress. For nuclear counterstains, DAPI-DNA or TOPRO3 was used (FIG. 1B). Note that TOPRO3 and nucBlue (Hoechst 33342), unlike DAPI, do not shift their emission wavelength in the presence of polyP.

First, the three polyP staining methods and their specificity were tested in mouse primary spinal cord astrocyte cultures. Astrocytes derived from mutant SOD1 (mutSOD1) mice were used, which showed significantly higher intracellular polyP staining signal compared to wild-type astrocytes (see below). Confocal microscopy revealed that the distribution of the polyP signal detected with recPPBD in primary spinal cord astrocytes was compartmentalized, with high-intensity puncta in the cytoplasm (middle images, FIG. 1B). Co-staining assays along with confocal microscopy further showed that the recPPBD-polyP signal co-localized with cytoplasmic DAPI-polyP (upper images, FIG. 1B) and JC-D8 in astrocytes (lower images, FIG. 1B). To confirm the specificity of recPPBD, astrocytes were infected with low titers of an AAV9 virus carrying the gene encoding the yeast exopolyphosphatase 1 (scPPX1) fused to GFP: this enzyme hydrolyzes polyP into inorganic phosphate (Pi). By contrast with yeast and bacterial PPX, the equivalent mammalian PPX enzyme has not been identified. As expected, the recPPBD signal was strongly decreased in the cytoplasm of GFP-positive astrocytes expressing scPPX1 as compared to neighboring GFP-negative (uninfected) astrocytes. In another specificity experiment, recPPBD, JC-D8 and DAPI were pre-incubated with an excess of a long synthetic polyphosphate polyPL (130 Pi chain length; 750 μg/ml for 30 min) before their addition to mutSOD1 astrocytes. Using such polyPL pre-absorption assays, it was found that the detection of endogenous polyP in mutSOD1 astrocytes was strongly reduced, indicating that recPPBD, JC-D8 and DAPI efficiently interacted with exogenous polyPL and hence prevented their binding to endogenous polyP. Together, these findings indicate that endogenous polyP in the cytoplasm of cultured astrocytes can be reliably detected by three independent polyP staining methods. A drawback of JC-D8- and DAPI-based approaches is that these dyes lose sensitivity after fixation, making the detection particularly challenging when the cells (such as neurons) exhibit low polyP content (data not shown).

Next, these three methods were used to assess intracellular polyP levels in 3-week-old primary astrocytes cultured at neonatal stages (P0-1) from the following well-studied transgenic ALS/FTD mouse models: (i) mutSOD1 ALS mice expressing human SOD1^{G93A} (Gurney et al., 1994), (ii) mutTDP43 ALS mice expressing human TARDBP^{A134T} (Wegorzewska et al., 2009), and (iii) mutC9ORF72 C9ALS-FTD mice carrying a bacterial artificial chromosome containing part of the human C9ORF72 gene (exon 1-6) with ˜500 G4C2 repeats in intron 1. The SOD1^{G93A} and TARDBP^{A314T} models recapitulate distinctive clinical motor deficits and histopathological features of ALS; the BAC transgenic C9ORF72 model does not develop weakness but does develop mild cognitive deficits and displays the molecular and pathological hallmarks of C9ALS-FTD (Jury et al., 2020; Peters et al., 2015). Given the diversity in the backgrounds of the ALS/FTD mice (see Methods), astrocytes from non-transgenic (NTg) littermates were used to generate the control astrocytes for each ALS/FTD model. Like astrocytes from wild-type mice, astrocytes from non-transgenic littermates of the mutSOD1, mutTDP43 and mutC9ORF72 mice revealed a weak polyP signal; for simplicity, only the image of a mutSOD1-NTg astrocyte is shown (FIG. 1C). Confocal microscopy revealed that recPPBD staining was substantially higher in mutSOD1, mutTDP43 and mutC9ORF72 astrocytes compared to their corresponding NTg astrocytes (FIG. 1C). PolyP staining assays with either JCD8 or DAPI-polyP also showed a marked increase in polyP fluorescence intensity in these ALS/FTD astrocytes compared to their NTg astrocytes. Quantification indicated that the average fluorescence intensity obtained with recPPBD (upper graph), JC-D8 (middle graph) and DAPI-polyP (lower graph) was approximately 2-fold higher in the cytoplasm of astrocytes that harbor mutSOD1 (FIG. 1D), mutTDP43 (FIG. 1E) or mutC9ORF72 (FIG. 1F), compared to their NTg controls. Intracellular polyP content was also analyzed in astrocytes from transgenic mice over-expressing wild-type human SOD1 gene (termed “wtSOD1”) and found to be comparable to that in astrocytes from NTg littermates (FIG. 10). This finding indicates that an increased intracellular polyP level in mutSOD1 astrocytes is specifically attributable to the human SOD1^{G93A} mutant protein, rather than to the overexpression of the human SOD1 protein.

Taken together, these experiments using three staining methods demonstrated that polyP is enriched in primary astrocyte cultures derived from three distinct and unrelated ALS/FTD mouse models, as compared to NTg control animals.

### Example 2: Primary Mouse ALS/FTD Astrocytes Release High Levels of polyP that is Deposited on Neuronal Plasma Membranes

Recent studies using co-cultures of neurons and glia have shown that polyP localizes within lysosomal vesicles that can be released from astrocytes into the extracellular space upon treatment with calcium ionophores, after which polyP can be subsequently taken up by neurons. In agreement with these findings, studies described herein showed by total internal reflection fluorescence (TIRF) microscopy that JCD8-labeled mutSOD1 and NTg astrocytes can release vesicular polyP after treatment with ionomycin, a calcium ionophore (data not shown). Labeling assays further showed that both synthetic and astrocyte-derived polyP (in ACM) adhere rapidly (≤5 min) to the plasma membrane of primary neurons in spinal cord cultures (FIGS. 2A-2B). A polyP deposition assay was used to gain insight into the level of polyP in astrocyte-conditioned media from the three ALS/FTD astrocytes described above; mutSOD1-ACM, mutTDP43-ACM and mutC9ORF72-ACM. It was previously shown that ACM-mutSOD1 (Fritz et al., 2013), ACM-mutTDP43 (Rojas et al., 2014) or ACM-mutC9ORF72 (Jury et al., 2020) are toxic to motoneurons, killing 40-50% of the cells even when the media were highly diluted (8-9 times). Accordingly, for the studies described herein (FIGS. 2, 5 and 6), ALS/FTD-ACMs were also diluted 8-9 fold (see Methods). As internal controls, ACM from NTg astrocytes (NTg-ACM; also diluted) and feeding medium (Ctrlmedium) were included and found to be innocuous for cultured motoneurons. As a positive control, 50-100 μM synthetic polyphosphate polyPL (130 Pi chain length) was used, as the rodent brain has been shown to contain 25-120 μM polyP (in terms of Pi residues), estimated by radioactive labeling studies (Kornberg et al., 1999; Kumble and Kornberg, 1995). Primary ventral spinal cord neuronal cultures were also generated, typically containing 8-10% motoneurons until 12 days in vitro (DIV) (Fritz et al., 2013). To visualize the morphology of neurons, cultures were infected with AAV2 virus carrying the gene encoding the fluorescent protein tdTomato under the neuronal synapsin promoter (AAV-synapsin-tdTomato). At 7-8 DIV, cultures were treated with ACMs or polyPL (50 μM) for 5 min and polyP detected by JC-D8 staining. JC-D8 signal on tdTomato-positive neurons, including motoneurons, was clearly detectable when exposed to ACM from mutSOD1, mutTDP43 or mutC9ORF72 astrocytes, relative to their corresponding control media (FIG. 2A). Quantification revealed that the average JC-D8-positive fluorescence intensity on tdTomato-positive spinal cord neurons was significantly higher when exposed to ALS/FTD-ACM compared to NTg controls, and that these intensities were comparable to those obtained when the cells were treated with 50 μM PolyPL (FIG. 2B). Taken together, the data obtained with the polyP deposition assay indicates that the level of polyP is increased in ALS/FTD-ACMs compared to control media.

Given that mutSOD1-ACM specifically kills MNs in spinal cord cultures, it was evaluated if polyP released by mutSOD1 astrocytes is preferentially targeting MNs versus interneurons (INs). A drawback of the JC-D8-based staining method is that this dye largely loses its fluorescent signal when cells are processed for immunostaining assays. Hence, the recPPBD probe was used to detect the polyP signal on, or in proximity to, membranes of MNs and INs exposed to mutSOD1-ACM (only limited recPPBD signal was detected in the absence of ACM, data not shown). Typically, cells in primary spinal cord cultures are fixed at 7 DIV and double immunostained for unphosphorylated neurofilament-H (SMI32) and MAP2 to identify MNs (SMI32⁺/MAP2⁺-cells) and INs (SMI32⁻/MAP2⁺-cells) (Fritz et al., 2013; Mishra et al., 2020; Nagai et al., 2007). MNs were also visualized by using an antibody against choline acetyltransferase (ChAT; FIG. 2C). Confocal images and subsequent quantification revealed that both MNs (ChAT⁺/MAP2⁺) and INs (ChAT⁻/MAP2⁺) exhibited comparable recPPBD signals when treated with ACM-mutSOD1 for 5 min (FIG. 2D). Similar results were obtained when MNs were identified with SMI32 staining (FIG. 2E).

Next, two approaches were used to quantify polyP levels in mutSOD1-ACM. Determining the polyP concentration in mammalian samples, particularly in ACM, is appealing but represents a unique challenge due to the low polyP concentration (in the M range versus mM range in yeast and bacteria) and long polyP size chains reported in brain tissue. Therefore, methods such as ³¹P NMR that require high sample concentrations (mM range) or electrospray ionization mass spectrometry (ESI-MS) that can determine very short-chain polyP are not suitable for mammalian samples (Christ et al., 2020). As an alternative, enzymatic assays have been used to measure the concentration of long-chain polyP in diverse types of organisms. These assays include three steps: i) extraction of polyP from the sample, ii) digestion of polyP with recombinant PPX (recPPX), either alone or in combination with recombinant pyrophosphatase 1 (recPPX/PPase), and iii) radioactive, colorimetric or fluorometric detection of the released Pi (Christ et al., 2020). In the rodent brain, radioactive (Kumble and Komberg, 1995) and fluorometric (Lee et al., 2018) methods detected 95 μM and 48 μM polyP (measured as Pi), respectively. To isolate and purify polyP from mutSOD1-ACM and NTg-ACM, a modified version of a phenol-chloroform (P/C) extraction protocol originally devised for yeast was developed (Christ and Blank, 2018); this initial extraction step followed by molecular weight cut-off filters is critical to remove large (e.g., RNA, DNA) and small (e.g., P3, ATP) P-containing molecules, respectively, that can interfere with the polyP detection. Next, polyP was enzymatically hydrolyzed with recPPX/PPase and the released Pi measured by a malachite-based colorimetric assay (Bru et al., 2016; Christ and Blank, 2018). Standard curves were generated using synthetic polyPL (dissolved in water) and Pi to then calculate the ACM polyP concentration and estimate the polyP recovery ratio (73±9%). Malachite quantification of P/C-extracted polyP (FIG. 2F) confirmed that mutSOD1-ACM (40±5 M) contained a ˜4.0-fold higher level of polyP (measured as Pi) compared to NTg-ACM (11±3 M). In an alternative assay, P/C-extracted polyP was not digested with PPX/PPase and the polyP polymer directly fluorometrical quantified using the JC-D8 probe. Quantification demonstrated that mutSOD1-ACM contained ˜1.4-fold higher levels of polyP-polymer compared to NTg-ACM (FIG. 2G). Together, and in agreement with the polyP deposition staining assay, two analytical strategies described herein demonstrate that there is a significantly higher polyP concentration in mutSOD1-ACM compared to control ACM.

### Example 3: PolyP is Enriched in Spinal Cord and Motor Cortex Sections of ALS/FTD Mouse Models

To determine whether the polyP level is also elevated within the spinal cord and motor cortex of the three ALS/FTD transgenic mice described above, recPPBD staining along with confocal microcopy was used. Tissue samples (lumbar 4-6) were examined including those derived from symptomatic mutSOD1 (4-month-old), mutTDP43 (2-month-old) and mutC9ORF72 (9-month-old) mice, and NTg littermates were used as controls. Fluorescent staining of fixed sections was performed using the polyP probe recPPBD and an antibody that targets the glial fibrillary acidic protein (GFAP) to identify astrocytes. As expected, there was marked astrogliosis in ventral spinal cord of mutSOD1 animals. Astrocytes in mutSOD1 spinal cord, unlike those in control tissue, exhibited a strong recPPBD signal consisting mostly of high-intensity cytoplasmic puncta (FIG. 3A). The average recPPBD fluorescence intensity signal in the soma of GFAP-positive cells was approximately 5-fold higher in mutSOD1 tissue compared to controls (FIG. 3C, top). Evident recPPBD labeling in the mutSOD1 spinal cord samples was also detected outside GFAP-positive cells as well as in GFAP-negative cells. Presence of polyP in motoneurons was analyzed by double immunofluorescence staining with recPPBD and the neuronal marker NeuN. Motoneurons were easily identified on the basis of their large size and localization in the ventral horn of the spinal cord (FIG. 3B). As expected for this ALS mouse model symptomatic stage, there was a significant loss of NeuN-positive motoneurons in the ventral spinal cord. It was found that the remaining atrophied NeuN-positive motoneurons revealed significantly higher cytoplasmic recPPBD immunoreactivity than controls (FIG. 3C, bottom). Similar staining assays in spinal cord sections of mutC9ORF72 (FIG. 3D) and mutTDP43 (FIG. 3E) mice also revealed significantly increased recPPBD immunoreactivity in GFAP-positive and NeuN-positive cells. Another set of experiments, performed with JC-D8 live staining, also revealed higher polyP labeling in NeuN-positive and -negative cells in the motor cortex of mutSOD1 and mutC9ORF72 animals relative to NTg littermates and transgenic wtSOD1 mice (data not shown).

### Example 4: ALS Patients Exhibit polyP Enrichment in Spinal Cord Sections and in CSF

To address whether polyP is enriched in fALS (carrying mutSOD1 and mutC9ORF72) and sALS patients (Table 1), immunohistochemical staining assays with recPPBD were performed on formalin-fixed paraffin-embedded section through the ventral spinal cord. Based on the morphology and size of cells (Cooper-Knock et al., 2012; Forsberg et al., 2011; Kiernan and Hudson, 1991), large MN-like cells and smaller non-neuronal glia-like cells were identified throughout the ventral cord gray matter of control human spinal cord sections. The spinal cord ventral horn of most, but not all (e.g., sALS #8), ALS patients showed fewer MNs than controls; residual MN-like cells were typically atrophied (FIG. 4B and FIG. 4E). In spinal cord sections from both control (FIG. 4A) and ALS (FIG. 4B) subjects, recPPBD immunostaining was detected intracellularly and extracellularly. Importantly, the intensity of the polyP staining was higher in ALS patients and quantification revealed that glia-like cells (FIG. 4C), but not MN-like cells (FIG. 4D), from ALS patients displayed a significantly higher recPPBD immunoreactivity relative to control subjects. These findings indicate that polyP enrichment in spinal and cortical astrocytes represents a novel hallmark of ALS/FTD.

TABLE 1
Patient information.

| Control cases | | | ALS cases | | | |
|---|---|---|---|---|---|---|
| | | SP | | Age | SP | |
| No | Age | zone | No | onset/death | Zone | Mutation |
| 1 | 77 | Lumbar | 1 | 33/34 | Lumbar | C9ORF72 |
| 2 | 76 | Lumbar | 2 | 43/47 | N/A | C9ORF72 |
| 3 | 44 | Lumbar | 3 | 22/27 | N/A | sALS |
| 4 | 70 | Lumbar | 4 | 54/56 | Thoracic | C9ORF72 |
| 5 | 50 | Lumbar | 5 | 67/74 | Cervical | C9ORF72 |
| | | | 6 | 53/55 | Thoracic | SOD1-A4V |
| | | | 7 | 79/79 | Thoracic | sALS |
| | | | 8 | 77/77 | Lumbar | sALS |
| | | | 9 | 64/66 | Lumbar | C9ORF72 |

Next, it was assessed whether polyP concentrations are elevated in the cerebrospinal fluid (CSF) of sALS and fALS patients (Table 2). To quantify CSF polyP concentration, a modified version of a silica extraction protocol originally devised for mammalian tissues was developed (Lee et al., 2018). Specifically, the polyP from CSF (1 ml) of 16 ALS patients and of 15 control subjects was extracted with silica columns and PPX/PPase-digested Pi was measured with malachite. Importantly, quantification revealed that the polyP concentration in ALS patient CSF (0.188±0.014 μM) contained ˜1.5-fold higher level of polyP (measured as Pi) compared to control subject CSF (0.123±0.015 μM), at a 75±3% recovery ratio with artificial CSF (FIG. 4F). Importantly, these findings indicate that polyP enrichment in CSF may represent a novel biomarker of ALS.

TABLE 2
Patient information.

| | | | ALS cases | | | | |
|---|---|---|---|---|---|---|---|
| Control cases | | | | Age | | Disease | |
| No | Age | Gender | No | Onset | Gender | Type | Mutation |
| 1 | 49 | F | 1 | 65 | M | Familial | C9ORF72 |
| 2 | 53 | M | 2 | 60 | F | Sporadic | sALS |
| 3 | 38 | F | 3 | 65 | F | Sporadic | sALS |
| 4 | 50 | F | 4 | 35 | F | Unknown | N/A |
| 5 | 43 | F | 5 | 74 | F | Sporadic | sALS |
| 6 | 48 | F | 6 | 63 | F | Familial | C9ORF72 |
| 7 | 31 | F | 7 | 47 | M | Sporadic | sALS |
| 8 | 28 | M | 8 | 68 | M | Unknown | N/A |
| 9 | 24 | M | 9 | 53 | M | Sporadic | sALS |
| 10 | 40 | F | 10 | 60 | F | Familial | C9ORF72 |
| 11 | 23 | M | 11 | 43 | M | Sporadic | sALS |
| 12 | 26 | M | 12 | 35 | M | Sporadic | sALS |
| 13 | 56 | M | 13 | 25 | M | Familial | TARDBP |
| 14 | 35 | M | 14 | 46 | M | Sporadic | sALS |
| 15 | 27 | F | 15 | 33 | M | Familial | SOD1 |
| | | | 16 | 54 | M | Sporadic | sALS |

### Example 5: Exposure of Wild-Type Spinal Cord Neurons to polyP Reproduces the Toxic Effects of mutSOD1-ACM

Our earlier experiments demonstrated that application of mutSOD1-ACM to cultures of wildtype spinal cord neurons rapidly induces hyperexcitability, leading to excessive Ca²⁺ influx and motoneuronal death within a few days (Fritz et al., 2013; Rojas et al., 2014). Therefore, it was next explored whether application of synthetic polyP mimics the toxicity of ACM derived from mutSOD1 astrocytes. Because the length of polyP can range from 50 to 800 residues in rodent tissue, synthetic polyP with varying polymer size ranges was used: polyPs (short; 14 Pi chain length); polyPM (medium; 60 Pi chain length); polyPL (large; 130 Pi chain length). These synthetic polyP polymers were applied to 3 DIV primary spinal cord cultures, at concentrations up to 100 μM, to mimic the conditions shown in FIGS. 2A-2B and found in CNS tissue. The survival of motoneurons and interneurons was assessed at 7 DIV as reported. Cultures were fixed and double-labeled with an antibody against MAP2 to identify all neurons (motoneurons and interneurons) and an antibody against SMI-32 to selectively identify motoneurons (FIG. 5A, image). Quantification of motoneuron (SMI32+/MAP2+) survival revealed that a single application of 100 μM polyPL killed ˜40% of motoneurons, which is comparable to that seen with mutSOD1-ACM (FIG. 5A, graph). The toxic activity of the smaller synthetic polyP polymers, polyPs and polyPM, was less evident, especially at lower concentrations. Transcriptomic analyses further revealed that treatment of spinal cord cultures for 90 min with polyPL (100 μM) or mutSOD1-ACM leads to a common set of differentially expressed mRNAs (97 genes); functional annotation analysis (GO for biological process, molecular function, and cellular components) underscored that these genes are functionally enriched in inflammatory-related events (FIG. 5B and Table 3). Whole-cell patch clamp electrophysiological recordings and Ca²⁺ imaging (measured with fura-2) of primary spinal cord neurons were used to determine whether polyP_{L} (100 μM) mimics the membrane action potentials and cyoplasmic Ca²⁺ dynamics induced by mutSOD1-ACM. Application of synthetic polyP to DRG and hippocampal neurons has been shown to enhance neuronal excitability and Ca²⁺ signaling. In line with this report, it was found that polyPL rapidly and significantly increased the intracellular Ca²⁺ transients (FIG. 5C) as well as the firing of spontaneous action potentials (FIG. 5D), to an extent that is comparable to that induced by mutSOD1-ACM.

TABLE 3
A common set of transcripts altered
in Synthetic polyPL and mutSOD1-ACM

| | | | | |
|---|---|---|---|---|
| Cxcl1 | Gal | Itgam | Bcl2a1 | Gfap |
| Cxcl2 | Pgf | Angptl4 | Il6r | Nfkbie |
| Tnf | Zc3h12a | Bhlhe40 | Mir331 | LOC100910973 |
| Ccl20 | Mir124-1 | Nfkb2 | Itpkc | Mgst1 |
| Lif | Gbp2 | Fst | Fcgr2a | Sfrp2 |
| Ccl4 | Tfpi2 | Adamts1 | Sphk1 | Dcn |
| Icam1 | Gldn | Cyth4 | Sbno2 | Mir760 |
| Nfkbiz | Rhbdf2 | Irf1 | Ets1 | Lum |
| Gpr84 | Cebpd | Trim47 | Map2k3 | Krt8 |
| Pkp1 | Itga5 | Cxcl16 | Nfkb1 | |

These data show that addition of polyPL to spinal cord neurons reproduces the effects of ALS-ACM by both increasing excitability and Ca²⁺ transients, along with the altered expression of inflammatory-related gene transcripts, effects that can trigger motoneuron death.

### Example 6: Reduction of polyP in mutSOD1 Astrocytes, or Sequestering polyP in ALS/FTD-ACM, Restores Intracellular Ca²⁺ Transients and Prevents Motoneuron Death

To confirm that polyP is a toxic component in mutSOD1-ACM, the toxicity of ACM from mutSOD1 astrocytes was assayed after reducing polyP within the ALS astrocytes and neutralizing polyP in the corresponding ACM. To reduce polyP in mutSOD1 astrocytes, cells were transduced with AAV9 vectors carrying different versions of the scPPX1 gene, fused to GFP, and driven by a CMV promoter. The experimental approach included the following two constructs: (i) PPX-GFP, where the wild-type enzyme is ubiquitously expressed in the cytoplasm; and (ii) PPX-PLong-GFP, where PPX-GFP was genetically fused to the membrane-permeant peptide PTEN-Long-specific region. This latter chimeric protein was designed to impart PPX-GFP with a mechanism for secretion and re-uptake into cells, and thus establish a bystander effect. Three days after mutSOD1 astrocytes were infected with AAV9 vectors, ACM was collected and added to the primary spinal cord cultures. It was found that ACM derived from mutSOD1 astrocytes transduced with the PPX-coding constructs, particularly with PPX-PLong-GFP, was significantly less toxic to motoneurons than ACM from untreated mutSOD1 astrocytes (FIG. 6A). Next, the recPPBD protein was applied to mutSOD1-ACM, aiming to sequester and inactivate extracellular polyP released by mutSOD1 astrocytes and hence preventing its toxic effects on motoneurons. Pretreatment of mutSOD1-ACM with recPPBD (20-40 μg/ml) before applying it to spinal cord cultures effectively prevented, in a concentration-dependent manner, the death of motoneurons by mutSOD1-ACM (FIG. 6B). This approach was further explored using synthetic nano-sized tree-like branched polycationic compounds to sequester extracellular polyP. In particular, studies described herein evaluated poly-amidoamine (PAMAM) and universal heparin reversal agents (UHRAs), which are nano-sized cationic branched polymers shown to efficiently bind and neutralize polyP (Travers et al., 2014; Smith et al., 2012). Application of the dendrimer G4-PAMAM-NH2 (14 kDa, 1 μg/mL), containing an ethylenediamine core and 64 positively charged primary NH₂ groups on the surface, significantly reduced motoneuron death when applied together with mutSOD-ACM to spinal cord cultures (FIG. 6C). Additionally, studies described herein evaluated UHRA9 (16 kDa) and UHRA10 (10 kDa) which were engineered to contain 16 and 11 polyP-binding groups (with each containing positively charged tertiary amines), respectively (Travers et al., 2014). Motoneuron death was completely abolished when UHRA9 (250 nM) or UHRA10 (250-500 nM) were added to the mutSOD-ACM (FIG. 6D). The number of events of the intracellular Ca²⁺ transients measured with GCaMP6 reverted to control values when UHRA10 was added to mutSOD-ACM (FIG. 6E). It was also assessed whether the toxic component present in mutTDP43-ACM and mutC9ORF72-ACM can be inhibited by the treatment with these nano-sized poly-cationic compounds. Treatment of mutTDP43-ACM (FIGS. 6F-6G) or mutC9ORF72-ACM (FIGS. 6H-6I) with UHRA10 (500 nM) significantly reduced the numbers of ACM-induced intracellular Ca²⁺ transient events in neurons and protected motoneurons from death. It was also determined whether MN death can be prevented by degrading or neutralizing polyP in ALS/FTD-ACM. Experimental results provided herein showed that pre-treatment of mutSOD1-ACM with the the exopolyphosphatases recPPX/PPase or CIP significantly protected MNs from death (FIG. 6J).

Taken together, these findings show that the polyP present in astrocyte-conditioned media derived from diverse ALS/FTD astrocytes is a critical neurotoxic factor in non-cell autonomous motoneuron degeneration.

### Example 7: Human mutTDP43 Astrocytes and Derived ACM is Toxic to MNs and Exhibit a Higher polyP Concentration

It was important to confirm that the toxic effect shown by polyP-containing mouse ACM on MNs is also conserved in ACM produced by human astrocytes. Hence, ACM from human astrocytes derived from induced pluripotent stem cells (iPSCs) from an ALS/FTD subject carrying a A90V mutation in TARDBP (mutTDP43) and a healthy family member (control) were evaluated (Zhang et al., 2013). An efficient mature astrocyte phenotype was achieved for both control and mutTDP43 patient-derived iPSCs, a stage that is characterized by a high percentage of cells expressing the following mature astrocyte markers (Roybon et al., 2013): Cx43 (>91% positive cells), EAAT2 (>93%) and s1000 (>78%). Comparable to mouse mutTDP43 astrocytes (FIG. 1E), confocal microscopy and subsequent quantification demonstrated that the average fluorescence intensity obtained with the recPPBD-based labeling was ˜2-fold higher in the cytoplasm of mutTDP43 patient astrocytes compared to control subject astrocytes (FIGS. 7A-7B). Additionally, it was also found that MN death was prevented when mutTDP43-ACM was pre-treated with recPPX/PPase or CIP (FIG. 7C). JC-D8 quantification of P/C-extracted polyP (68±8% recovery ratio) further confirmed that human mutTDP43-ACM contained a ˜2-fold higher level of polyP-polymer compared to control (FIG. 7D).

### Example 8: Chronic Treatment of mutSOD1 Transgenic Drosophila Model with the polyP-Neutralizing Molecule UHRA10 Reduces Locomotion Deficits and Prolongs Mean Lifespan

To evaluate the polyP neutralizing molecule UHRA10 as a potential therapy targeting non-cell autonomous toxicity in ALS in vivo, the GAL4-UAS system was used to restrict human SOD1^{A4V} expression to glial cells under the promoter repo (mutSOD1) (FIGS. 8A-8B). As experimental background controls, in addition to wild-type flies (Ctrl), Drosophila lines were established that expressed GFP (GFP) or human wild-type SOD1 (wtSOD1) with the repo glial driver. Staining with JC-D8 revealed higher polyP labeling in the brains of mutSOD1 flies relative to wtSOD1 flies (FIG. 8C). Also, it was found that the selective expression in glial cells of mutSOD1, but not GFP or wtSOD1, induced progressive climbing deficits, which became particularly evident in 30 days old adult flies (FIGS. 8D-8E). The locomotion dysfunction was accompanied by decreased mean survival of transgenic mutSOD1 flies (FIG. 8F). These results further extend studies in rodents in which motoneuron degeneration, locomotor deficits, and reduced survival of animals were found when the ALS-linked TDP43 mutant was selectively expressed in astrocytes, or when mutSOD1 or sALS progenitor cells—capable of differentiating into astrocytes in the spinal cord—were transplanted in acceptor wild-type rodents. Next, transgenic SOD1 fly lines were fed with UHRA10 (500 nM every other day from day 1 post-eclosion). It was found that the chronic UHRA10 treatment significantly improved climbing ability in 30 days old adult flies and increased mean survival (FIGS. 8E-8F).

These results indicate that selective expression of ALS-linked mutSOD1 in glial cells in Drosophila leads to locomotion dysfunction and reduced lifespan; mechanistically these results implicate polyP toxicity, which is ameliorated by chronic UHRA10 treatment in vivo.

### Example 9: PolyP Levels are Elevated in ALS/FTD Astrocytes Cultured from mutSOD1 and mutC9ORF72 Mice

This Example shows quantification of cytoplasmic polyP levels (relative a.u.) with the use of DAPI-polyP in astrocytes carrying mutSOD1 (mutSOD1) relative to both astrocytes derived from non-transgenic littermates (NTg) and those expressing human wild-type SOD1 (wtSOD1). As shown in FIG. 9, quantification of cytoplasmic polyP levels indicated that an increased intracellular polyP level in mutSOD1 astrocytes is specifically attributable to the human SOD1G93A mutant protein, rather than to the overexpression of the human SOD1 protein. These results also show that polyP is enriched in primary astrocyte cultures derived from transgenic mice over-expressing wild-type human SOD1 or mutSOD1, as compared to NTg control animals.

### Example 10: Treatment of mutSOD1 Mice with the polyP-Neutralizing Dendrimer G3-PAMAM-NH₂ or the polyP-Neutralizing Cation Polyamine Spermidine Prolongs Survival

In this Example, polyP neutralizing molecules including a dendrimer (e.g., G3-PAMAM-NH₂) and a cationic polyamine (e.g., spermidine) were evaluated as potential therapies for neurological disorders using a mouse model of ALS. To evaluate G3-PAMAM-NH₂, 11 mutSOD1 mice (7 males+4 females) were i.p. injected at postnatal day 60 (P60) with G3-PAMAM-NH₂ (15-20 μg G3-PAMAM-NH₂ per gram mouse; Sigma Cat. #412422) for 3 times a week till human end-stage. As controls, 11 mutSOD1 mice (7 males+4 females) were i.p. injected with saline and 8 non-transgenic (NTg) control littermate mice (4 males+4 females) were i.p. injected with G3-PAMAM-NH₂. As shown in FIG. 10A, mutSOD1 mice treated with G3-PAMAM-NH₂ exhibited a prolonged survival of about 2 weeks compared to mutSOD1 mice treated with saline. To evaluate spermidine, 12 mutSOD1 mice (4 males+8 females) were treated ad libitum at postnatal day 60 (P60) with 3 mM spermidine (spermidine trihydrochloride added to water; Sigma S2501) till human end-stage. As controls, 11 mutSOD1 mice (5 males+6 females) were untreated (water only) and 2 non-transgenic (NTg) control littermate mice (1 males+1 female) were treated with spermidine. As shown in FIG. 10B, mutSOD1 mice treated with G3-PAMAM-NH₂ exhibited a prolonged survival of about 1 week compared to untreated mutSOD1 mice.

Taken together, these results demonstrate that polyP-neutralizing molecules such as G3-PAMAM-NH₂ and spermidine provide therapeutic benefits against neurological disorders such as ALS.

### REFERENCES

Abramov, A. Y., Fraley, C., Diao, C. T., Winkfein, R., Colicos, M. A., Duchen, M. R., French, R. J., and Pavlov, E. (2007). Targeted polyphosphatase expression alters mitochondrial metabolism and inhibits calcium-dependent cell death. Proceedings of the National Academy of Sciences 104, 18091-18096.
Almeida, S., Zhang, Z., Coppola, G., Mao, W., Futai, K., Karydas, A., Geschwind, M. D., Tartaglia, M. C., Gao, F., Gianni, D., et al. (2012). Induced Pluripotent Stem Cell Models of Progranulin-Deficient Frontotemporal Dementia Uncover Specific Reversible Neuronal Defects. Cell Reports 2, 789-798.
Angelova, P. R., Agrawalla, B. K., Elustondo, P. A., Gordon, J., Shiba, T., Abramov, A. Y., Chang, Y.-T., and Pavlov, E. V. (2014). In SituInvestigation of Mammalian Inorganic Polyphosphate Localization Using Novel Selective Fluorescent Probes JC-D7 and JC-D8. ACS Chemical Biology 9, 2101-2110.
Angelova, P. R., Iversen, K. Z., Teschemacher, A. G., Kasparov, S., Gourine, A. V., and Abramov, A. Y. (2018). Signal transduction in astrocytes: Localization and release of inorganic polyphosphate. Glia 66, 2126-2136.
Aschar-Sobbi, R., Abramov, A. Y., Diao, C., Kargacin, M. E., Kargacin, G. J., French, R. J., and Pavlov, E. (2008). High Sensitivity, Quantitative Measurements of Polyphosphate Using a New DAPI-Based Approach. J Fluoresc 18, 859-866.
Baev, A. Y., Angelova, P. R., and Abramov, A. Y. (2020). Inorganic polyphosphate is produced and hydrolyzed in FOF1-ATP synthase of mammalian mitochondria. Biochemical Journal 477, 1515-1524.
Birger, A., Ben-Dor, I., Ottolenghi, M., Turetsky, T., Gil, Y., Sweetat, S., Perez, L., Belzer, V., Casden, N., Steiner, D., et al. (2019). Human iPSC-derived astrocytes from ALS patients with mutated C9ORF72 show increased oxidative stress and neurotoxicity. EBioMedicine 50, 274-289.
Bondy-Chorney, E., Abramchuk, I., Nasser, R., Holinier, C., Denoncourt, A., Baijal, K., McCarthy, L., Khacho, M., Lavallee-Adam, M., and Downey, M. (2020). A Broad Response to Intracellular Long-Chain Polyphosphate in Human Cells. Cell Reports 33, 108318.
Bru, S., Jimenez, J., Canadell, D., Arino, J., and Clotet, J. (2017). Improvement of biochemical methods of polyP quantification. Microb Cell 4, 6-15.
Christ, J. J., and Blank, L. M. (2018). Analytical polyphosphate extraction from Saccharomyces cerevisiae. Analytical Biochemistry 563, 71-78.
Christ, J. J., Willbold, S., and Blank, L. M. (2020). Methods for the Analysis of Polyphosphate in the Life Sciences. Anal. Chem. 92, 4167-4176.
Clement, A. M., Nguyen, M. D., Roberts, E. A., Garcia, M. L., Boillee, S., Rule, M., McMahon, A. P., Doucette, W., Siwek, D., Ferrante, R. J., et al. (2003). Wild-Type Nonneuronal Cells Extend Survival of SOD1 Mutant Motor Neurons in ALS Mice. Science 302, 113-117.
Cooper-Knock, J., Hewitt, C., Highley, J. R., Brockington, A., Milano, A., Man, S., Martindale, J., Hartley, J., Walsh, T., Gelsthorpe, C., et al. (2012). Clinico-pathological features in amyotrophic lateral sclerosis with expansions in C9ORF72. Brain 135, 751-764.
Cremers, C. M., Knoefler, D., Gates, S., Martin, N., Dahl, J.-U., Lempart, J., Xie, L., Chapman, M. R., Galvan, V., Southworth, D. R., et al. (2016). Polyphosphate: A Conserved Modifier of Amyloidogenic Processes. Molecular Cell 63, 768-780.
Desfougères, Y., Saiardi, A., and Azevedo, C. (2020). Inorganic polyphosphate in mammals: where's Wally?Biochemical Society Transactions 48, 95-101.
Devlin, A.-C., Burr, K., Borooah, S., Foster, J. D., Cleary, E. M., Geti, I., Vallier, L., Shaw, C. E., Chandran, S., and Miles, G. B. (2015). Human iPSC-derived motoneurons harbouring TARDBP or C9ORF72 ALS mutations are dysfunctional despite maintaining viability. Nature Communications 6, 5999.
di Domenico, A., Carola, G., Calatayud, C., Pons-Espinal, M., Muñoz, J. P., Richaud-Patin, Y., Fernandez-Carasa, I., Gut, M., Faella, A., Parameswaran, J., et al. (2019). Patient-Specific iPSC-Derived Astrocytes Contribute to Non-Cell-Autonomous Neurodegeneration in Parkinson's Disease. Stem Cell Reports 12, 213-229.
Di Giorgio, F. P., Carrasco, M. A., Siao, M. C., Maniatis, T., Eggan, K. (2007). Non-cell autonomous effect of glia on motor neurons in an embryonic stem cell-based ALS model. Nature Neuroscience 10, 608-614.
Forsberg, K., Andersen, P. M., Marklund, S. L., and Brannstrom, T. (2011). Glial nuclear aggregates of superoxide dismutase-1 are regularly present in patients with amyotrophic lateral sclerosis. Acta Neuropathologica 121, 623-634.
Fox, L. J., Richardson, R. M., and Briscoe, W. H. (2018). PAMAM dendrimer—cell membrane interactions. Adv Colloid Interface Sci 257, 1-18.
Fritz, E., Izaurieta, P., Weiss, A., Mir, F. R., Rojas, P., Gonzalez, D., Rojas, F., Brown, R. H., Madrid, R., and Zundert, B. van (2013). Mutant SOD1-expressing astrocytes release toxic factors that trigger motoneuron death by inducing hyperexcitability. Journal of Neurophysiology 109, 2803-2814.
Geevasinga, N., Menon, P., Ozdinler, P. H., Kiernan, M. C., and Vucic, S. (2016). Pathophysiological and diagnostic implications of cortical dysfunction in ALS. Nature Reviews Neurology 12, 651-661.
Gurney, M., Pu, H., Chiu, A., Canto, M. D., Polchow, C., Alexander, D., Caliendo, J., Hentati, A., Kwon, Y., Deng, H., et al. (1994). Motor neuron degeneration in mice that express a human Cu,Zn superoxide dismutase mutation. Science 264, 1772-1775.
Guttenplan, K. A., Weigel, M. K., Adler, D. I., Couthouis, J., Liddelow, S. A., Gitler, A. D., and Barres, B. A. (2020). Knockout of reactive astrocyte activating factors slows disease progression in an ALS mouse model. Nat Commun 11, 3753.
Haidet-Phillips, A. M., Hester, M. E., Miranda, C. J., Meyer, K., Braun, L., Frakes, A., Song, S., Likhite, S., Murtha, M. J., Foust, K. D., et al. (2011). Astrocytes from familial and sporadic ALS patients are toxic to motor neurons. Nature Biotechnology 29, 824-828.
Hallmann, A. L., Aranzo-Bravo, M. J., Mavrommatis, L., Ehrlich, M., Ropke, A., Brockhaus, J., Missler, M., Sterneckert, J., Scholer, H. R., Kuhlmann, T., Zaehres, H., Hargus, G. (2017). Astrocyte pathology in a human neural stem cell model of frontotemporaldementia caused by mutant TAU protein. Scientific Reports 7, 42991.
Hermosilla, T., Encina, M., Morales, D., Moreno, C., Conejeros, C., Alfaro-Valdes, H. M., Lagos-Meza, F., Simon, F., Altier, C., and Varela, D. (2017). Prolonged AT1R activation induces CaV1.2 channel internalization in rat cardiomyocytes. Sci Rep 7, 10131.
Holmström, K. M., Marina, N., Baev, A. Y., Wood, N. W., Gourine, A. V., and Abramov, A. Y. (2013). Signalling properties of inorganic polyphosphate in the mammalian brain. Nature Communications 4, 1362.
Hopkins, B. D., Fine, B., Steinbach, N., Dendy, M., Rapp, Z., Shaw, J., Pappas, K., Yu, J. S., Hodakoski, C., Mense, S., et al. (2013). A Secreted PTEN Phosphatase That Enters Cells to Alter Signaling and Survival. Science 341, 399-402.
Ikiz, B., Alvarez, M. J., Ré, D. B., Le Verche, V., Politi, K., Lotti, F., Phani, S., Pradhan, R., Yu, C., Croft, G. F., et al. (2015). The Regulatory Machinery of Neurodegeneration in In Vitro Models of Amyotrophic Lateral Sclerosis. Cell Reports 12, 335-345.
Jain, S., Pitoc, G. A., Holl, E. K., Zhang, Y., Borst, L., Leong, K. W., Lee, J., and Sullenger, B. A. (2012). Nucleic acid scavengers inhibit thrombosis without increasing bleeding. Proceedings of the National Academy of Sciences 109, 12938-12943.
Jury, N., Abarzua, S., Diaz, I., Guerra, M., Ampuero, E., Martinez, P., Cubillos, P., Herrera-Soto, A., Arredondo, C., Rojas, F., et al. (2020). Widespread loss of the silencing epigenetic mark H3K9me3 in astrocytes and neurons along with hippocampal-dependent cognitive impairment in C9orf72 BAC transgenic mice. Clinical Epigenetics 12, 32.
Kiernan, J. A., and Hudson, A. J. (1991). Changes in sizes of cortical and lower motor neurons in amyotrophic lateral sclerosis. Brain 114, 843-853.
Kornberg, A., Rao, N. N., and Ault-Riché, D. (1999). Inorganic Polyphosphate: A Molecule of Many Functions. Annu. Rev. Biochem. 68, 89-125.
Kumble, K. D., and Kornberg, A. (1995). Inorganic Polyphosphate in Mammalian Cells and Tissues. Journal of Biological Chemistry 270, 5818-5822.
Lee, W. D., Gawri, R., Shiba, T., Ji, A.-R., Stanford, W. L., and Kandel, R. A. (2018). Simple Silica Column-Based Method to Quantify Inorganic Polyphosphates in Cartilage and Other Tissues. CARTILAGE 9, 417-427.
Linden, M. A., Sedgewick, G. J., and Ericson, M. (2015). An Innovative Method for Obtaining Consistent Images and Quantification of Histochemically Stained Specimens. J Histochem Cytochem. 63, 233-243.
Lepore, A. C., Rauck, B., Dejea, C., Pardo, A. C., Rao, M. S., Rothstein, J. D., and Maragakis, N.J. (2008). Focal transplantation-based astrocyte replacement is neuroprotective in a model of motor neuron disease. Nature Neuroscience 11, 1294-1301.
Liddelow, S. A., Guttenplan, K. A., Clarke, L. E., Bennett, F. C., Bohlen, C. J., Schirmer, L., Bennett, M. L., Munch, A. E., Chung, W.-S., Peterson, T. C., et al. (2017). Neurotoxic reactive astrocytes are induced by activated microglia. Nature 541, 481-487.
Ling, S.-C., Polymenidou, M., and Cleveland, D. W. (2013). Converging Mechanisms in ALS and FTD: Disrupted RNA and Protein Homeostasis. Neuron 79, 416-438.
Lee, W. D., Gawri, R., Shiba, T., Ji, A.-R., Stanford, W. L., and Kandel, R. A. (2018). Simple Silica Column-Based Method to Quantify Inorganic Polyphosphates in Cartilage and Other Tissues. CARTILAGE 9, 417-427.
Mishra, V., Re, D. B., Le Verche, V., Alvarez, M. J., Vasciaveo, A., Jacquier, A., Doulias, P.-T., Greco, T. M., Nizzardo, M., Papadimitriou, D., et al. (2020). Systematic elucidation of neuron-astrocyte interaction in models of amyotrophic lateral sclerosis using multi-modal integrated bioinformatics workflow. Nat Commun 11, 5579.
Maiolino, M., O'Neill, N., Lariccia, V., Amoroso, S., Sylantyev, S., Angelova, P. R., and Abramov, A. Y. (2019). Inorganic Polyphosphate Regulates AMPA and NMDA Receptors and Protects Against Glutamate Excitotoxicity via Activation of P2Y Receptors. J. Neurosci. 39, 6038-6048.
Malik, N., Wang, X., Shah, S., Efthymiou, A. G., Yan, B., Heman-Ackah, S., Zhan, M., and Rao, M. (2014). Comparison of the Gene Expression Profiles of Human Fetal Cortical Astrocytes with Pluripotent Stem Cell Derived Neural Stem Cells Identifies Human Astrocyte Markers and Signaling Pathways and Transcription Factors Active in Human Astrocytes. PLoS ONE 9, e96139.
Nagai, M., Re, D. B., Nagata, T., Chalazonitis, A., Jessell, T. M., Wichterle, H., and Przedborski, S. (2007). Astrocytes expressing ALS-linked mutated SOD1 release factors selectively toxic to motor neurons. Nature Neuroscience 10, 615-622.
Norris V, Reusch RN, Igarashi K, Root-Bernstein R. (2014) Molecular complementarity between simple, universal molecules and ions limited phenotype space in the precursors of cells. Biol Direct. 10:28.
Papadeas, S. T., Kraig, S. E., O'Banion, C., Lepore, A. C., and Maragakis, N.J. (2011). Astrocytes carrying the superoxide dismutase 1 (SOD1G93A) mutation induce wild-type motor neuron degeneration in vivo. Proceedings of the National Academy of Sciences 108, 17803-17808.
Peters, O. M., Cabrera, G. T., Tran, H., Gendron, T. F., McKeon, J. E., Metterville, J., Weiss, A., Wightman, N., Salameh, J., Kim, J., et al. (2015). Human C9ORF72 Hexanucleotide Expansion Reproduces RNA Foci and Dipeptide Repeat Proteins but Not Neurodegeneration in BAC Transgenic Mice. Neuron 88, 902-909.
Qian, K., Huang, H., Peterson, A., Hu, B., Maragakis, N.J., Ming, G., Chen, H., and Zhang, S.-C. (2017). Sporadic ALS Astrocytes Induce Neuronal Degeneration In Vivo. Stem Cell Reports 8, 843-855.
Ramirez-Jarquín, U. N., Rojas, F., van Zundert, B., and Tapia, R. (2017). Chronic infusion of SOD1 G93A astrocyte-secreted factors induces spinal motoneuron degeneration and neuromuscular dysfunction in healthy rats. Journal of Cellular Physiology 232, 2610-2615.
Rao, N. N., Gómez-Garcia, M. R., and Komberg, A. (2009). Inorganic Polyphosphate: Essential for Growth and Survival. Annual Review of Biochemistry 78, 605-647.
Re, D. B., Le Verche, V., Yu, C., Amoroso, M. W., Politi, K. A., Phani, S., Ikiz, B., Hoffmann, L., Koolen, M., Nagata, T., et al. (2014). Necroptosis Drives Motor Neuron Death in Models of Both Sporadic and Familial ALS. Neuron 81, 1001-1008.
Renton, A. E., Chiò, A., and Traynor, B. J. (2014). State of play in amyotrophic lateral sclerosis genetics. Nature Neuroscience 17, 17-23.
Rojas, F., Cortes, N., Abarzua, S., Dyrda, A., and Zundert, B. van (2014). Astrocytes expressing mutant SOD1 and TDP43 trigger motoneuron death that is mediated via sodium channels and nitroxidative stress. Frontiers in Cellular Neuroscience 8, 24.
Saito, K., Ohtomo, R., Kuga-Uetake, Y., Aono, T., and Saito, M. (2005). Direct Labeling of Polyphosphate at the Ultrastructural Level in Saccharomyces cerevisiae by Using the Affinity of the Polyphosphate Binding Domain of Escherichia coli Exopolyphosphatase. Applied and Environmental Microbiology 71, 5692-5701.
Saxena, S., and Caroni, P. (2011). Selective Neuronal Vulnerability in Neurodegenerative Diseases: from Stressor Thresholds to Degeneration. Neuron 71, 35-48.
Sepulveda, F. J., Bustos, F. J., Inostroza, E., Zniga, F. A., Neve, R. L., Montecino, M., and van Zundert, B. (2010). Differential Roles of NMDA Receptor Subtypes NR2A and NR2B in Dendritic Branch Development and Requirement of RasGRF1. Journal of Neurophysiology 103, 1758-1770.
Shaltouki, A., Peng, J., Liu, Q., Rao, M. S., and Zeng, X. (2013). Efficient Generation of Astrocytes from Human Pluripotent Stem Cells in Defined Conditions. STEM CELLS 31, 941-952.
Shoji, K. F., Abudara, V., Ezan, P., Amigou, E., Saéz PJ, Jiang, J. X., Naus, N.C., Saez, JC, Giaume, C. (2011). Amyloid 0-induced death in neurons involves glial and neuronal hemichannels. Journal of Neuroscience 31, 4962-4977.
Smith, S. A., Choi, S. H., Collins, J. N. R., Travers, R. J., Cooley, B. C., and Morrissey, J. H. (2012). Inhibition of polyphosphate as a novel strategy for preventing thrombosis and inflammation. Blood 120, 5103-5110.
Smith, S. A., and Morrissey, J. H. (2007). Sensitive fluorescence detection of polyphosphate in polyacrylamide gels using 4′,6-diamidino-2-phenylindol. Electrophoresis 28, 3461-3465.
Stotz, S. C., Scott, L. O., Drummond-Main, C., Avchalumov, Y., Girotto, F., Davidsen, J., Gómez-Gárcia, M. R., Rho, J. M., Pavlov, E. V., and Colicos, M. A. (2014). Inorganic polyphosphate regulates neuronal excitability through modulation of voltage-gated channels. Molecular Brain 7, 42.
Stoica, L., Todeasa, S. H., Cabrera, G. T., Salameh, J. S., ElMallah, M. K., Mueller, C., Brown, R. H., and Sena-Esteves, M. (2016). Adeno-associated virus-delivered artificial microRNA extends survival and delays paralysis in an amyotrophic lateral sclerosis mouse model: AAV9-RNAi for ALS Gene Therapy. Ann Neurol. 79, 687-700.
Tijssen, J. P. F., Beekes, H. W., and Steveninck, J. V. (1982). Localization of polyphosphates in Saccharomyces fragilis, as revealed by 4′,6-diamidino-2-phenylindole fluorescence. Biochimica et Biophysica Acta (BBA)—Molecular Cell Research 721, 394-398.
Taylor, J. P., Brown, R. H., and Cleveland, D. W. (2016). Decoding ALS: from genes to mechanism. Nature 539, 197-206.
Tong, J., Huang, C., Bi, F., Wu, Q., Huang, B., Liu, X., Li, F., Zhou, H., and Xia, X.-G. (2013). Expression of ALS-linked TDP-43 mutant in astrocytes causes non-cell-autonomous motor neuron death in rats. The EMBO Journal 32, 1917-1926.
Travers, R. J., Shenoi, R. A., Kalathottukaren, M. T., Kizhakkedathu, J. N., and Morrissey, J. H. (2014). Nontoxic polyphosphate inhibitors reduce thrombosis while sparing hemostasis. Blood 124, 3183-3190.
Van Harten, A. C. M., Phatnani, H., and Przedborski, S. (2021). Non-cell-autonomous pathogenic mechanisms in amyotrophic lateral sclerosis. Trends in Neurosciences 44, 658-668.
Varcianna, A., Myszczynska, M. A., Castelli, L. M., O'Neill, B., Kim, Y., Talbot, J., Nyberg, S., Nyamali, I., Heath, P. R., Stopford, M. J., et al. (2019). Micro-RNAs secreted through astrocyte-derived extracellular vesicles cause neuronal network degeneration in C9orf72 ALS. EBioMedicine 40, 626-635.
Wainger, B. J., Kiskinis, E., Mellin, C., Wiskow, O., Han, S. S. W., Sandoe, J., Perez, N. P., Williams, L. A., Lee, S., Boulting, G., et al. (2014). Intrinsic Membrane Hyperexcitability of Amyotrophic Lateral Sclerosis Patient-Derived Motor Neurons. Cell Reports 7, 1-11.
Wang, L., Gutmann, D. H., and Roos, R. P. (2011). Astrocyte loss of mutant SOD1 delays ALS disease onset and progression in G85R transgenic mice. Human Molecular Genetics 20, 286-293.
Urushitani, M., Sik, A., Sakurai, T., Nukina, N., Takahashi, R., and Julien, J.-P. (2006). Chromogranin-mediated secretion of mutant superoxide dismutase proteins linked to amyotrophic lateral sclerosis. Nat Neurosci 9, 108-118.
Wegorzewska, I., Bell, S., Cairns, N.J., Miller, T. M., and Baloh, R. H. (2009). TDP-43 mutant transgenic mice develop features of ALS and frontotemporal lobar degeneration. Proceedings of the National Academy of Sciences 106, 18809-18814.
Xie, L., and Jakob, U. (2019). Inorganic polyphosphate, a multifunctional polyanionic protein scaffold. J. Biol. Chem. 294, 2180-2190.
Yamanaka, K., Chun, S. J., Boillee, S., Fujimori-Tonou, N., Yamashita, H., Gutmann, D. H., Takahashi, R., Misawa, H., and Cleveland, D. W. (2008). Astrocytes as determinants of disease progression in inherited amyotrophic lateral sclerosis. Nature Neuroscience 11, 251-253.
Weismann, C. M., Ferreira, J., Keeler, A. M., Su, Q., Qui, L., Shaffer, S. A., Xu, Z., Gao, G., and Sena-Esteves, M. (2015). Systemic AAV9 gene transfer in adult GM1 gangliosidosis mice reduces lysosomal storage in CNS and extends lifespan. Hum. Mol. Genet. 24, 4353-4364.
Zhang, Z., Almeida, S., Lu, Y., Nishimura, A. L., Peng, L., Sun, D., Wu, B., Karydas, A. M., Tartaglia, M. C., Fong, J. C., et al. (2013). Downregulation of MicroRNA-9 in iPSC-Derived Neurons of FTD/ALS Patients with TDP-43 Mutations. PLoS ONE 8, e76055.
Zhu, C., Beck, M. V., Griffith, J. D., Deshmukh, M., and Dokholyan, N. V. (2018). Large SOD1 aggregates, unlike trimeric SOD1, do not impact cell viability in a model of amyotrophic lateral sclerosis. Proceedings of the National Academy of Sciences 115, 4661-4665.
van Zundert, B., Peuscher, M. H., Hynynen, M., Chen, A., Neve, R. L., Brown, R. H., Constantine-Paton, M., and Bellingham, M. C. (2008). Neonatal Neuronal Circuitry Shows Hyperexcitable Disturbance in a Mouse Model of the Adult-Onset Neurodegenerative Disease Amyotrophic Lateral Sclerosis. Journal of Neuroscience 28, 10864-10874.
van Zundert, B., Izaurieta, P., Fritz, E., and Alvarez, F. J. (2012). Early pathogenesis in the adult-onset neurodegenerative disease amyotrophic lateral sclerosis. Journal of Cellular Biochemistry 113, 3301-3312.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a neurological disorder in a subject, the method comprising administering to a subject in need thereof an effective amount of a polyphosphate (polyP)-neutralizing molecule.

2. The method of claim 1, wherein the polyP-neutralizing molecule is a protein, a nucleic acid encoding a protein, or a polymer.

3. The method of claim 2, wherein the protein is an exopolyphosphatase protein or a polyphosphate binding domain thereof or wherein the protein is an endopolyphosphatase protein or a polyphosphate binding domain thereof.

4.-5. (canceled)

6. The method of claim 3, wherein the exopolyphosphatase protein comprises an amino acid sequence that is at least 95% identical to SEQ ID NO: 1, 2, 3, or 4, or to amino acids 306 to 513 of SEQ ID NO: 2.

7.-21. (canceled)

22. The method of claim 2, wherein the nucleic acid encoding a protein is comprised in a vector.

23. The method of claim 22, wherein the vector is a viral vector.

24. The method of claim 23, wherein the viral vector is selected from the group consisting of a retroviral vector, a lentiviral vector, an adenoviral vector, an adeno-associated viral vector, a herpes simplex viral vector, and a vaccinia viral vector.

25. The method of claim 2, wherein the polymer comprises a dendrimer, a polyamine, or both.

26. The method of claim 25, wherein the dendrimer comprises a polyamidoamine (PAMAM) dendrimer, a polypropylamine (POPAM) dendrimer, a polypropyleneimine (PPI) dendrimer, a polyethylenimine (PEI) dendrimer, a polyarylether (PAE) dendrimer, a polylysine dendrimer, a polyester dendrimer, an iptycene dendrimer, an aliphatic poly(ether) dendrimer, an aromatic polyether dendrimer, an universal heparin reversal agent (UHRA), or a combination thereof.

27. The method of claim 26, wherein the PAMAM dendrimer comprises a generation 3 (G3) PAMAM dendrimer, a generation 4 (G4) PAMAM dendrimer, or a generation 5 (G5) PAMAM dendrimer.

28. The method of claim 26, wherein the UHRA comprises UHRA-8, UHRA-9, UHRA-10, UHRA-14, or a combination thereof.

29. The method of claim 25, wherein the polyamine comprises spermidine, norspermidine, spermine, norspermine, putrescine, cadaverine, polyethylenimine (PEI), polybrene, poly-D-lysine, poly-L-lysine, poly-L-arginine, surfen, protamine, diethylenetriamine (DETA), triethylenetetramine (TETA), or a combination thereof.

30. The method of claim 1, wherein the polyP-neutralizing molecule is formulated in a pharmaceutical composition, which further comprises a pharmaceutically acceptable carrier.

31. The method of claim 1, wherein the subject is a human patient having a neurological disorder.

32. The method of claim 1, wherein the neurological disorder is a stroke, a brain injury, neuroinflammation, a neurodegenerative disease, or a combination thereof.

33. The method of claim 32, wherein the neurodegenerative disease is selected from the group consisting of amyotrophic lateral sclerosis (ALS), frontotemporal dementia (FTD), ALS and FTD (ALS-FTD), Parkinson's disease (PD), and Huntington's disease (HD).

34. The method of claim 1, further comprising administering to the subject an additional therapeutic agent.

35. A method for diagnosing a subject as having a neurological disorder, the method comprising:
detecting a level of polyphosphate (polyP) in a sample from a subject, and
comparing the level of polyP in the sample to a reference level, wherein presence of a level of polyP in the sample that is above the reference level indicates that the subject has a neurological disorder characterized by polyP-induced neurotoxicity.

36. The method of claim 35, wherein the sample comprises cerebrospinal fluid (CSF) or blood.

37. The method of claim 35, further comprising administering to a subject in need thereof an effective amount of a polyP-neutralizing molecule, wherein the polyP-neutralizing molecule is a protein, a nucleic acid encoding a protein, or a polymer.

38. (canceled)
